(19) United States
(12) Patent Application Publication
(10) Pub. No.: US 2019/0233861 A1
(43) Pub. Date: Aug. 1, 2019

(54) PROCESS FOR DEGRADING A POLYSACCHARIDE EMPLOYING A LYTIC POLYSACCHARIDE MONOOXYGENASE

(71) Applicants: NORWEGIAN UNIVERSITY OF LIFE SCIENCES, Ås (NO); INSTITUT NATIONAL DE LA RECHERCHE AGRONOMIQUE, Paris (FR)

(72) Inventors: Bastien BISSARO, Ås (NO); Vincent EIJSINK, As (NO); Gustav VAAJE-KOLSTAD, Ås (NO)

(73) Assignees: NORWEGIAN UNIVERSITY OF LIFE SCIENCES, Ås (NO); INSTITUT NATIONAL DE LA RECHERCHE AGRONOMIQUE, Paris (FR)

(21) Appl. No.: 16/337,624

(22) PCT Filed: Sep. 29, 2017
(86) PCT No.: PCT/EP2017/074904

§ 371 Date: Mar. 28, 2019

(30) Foreign Application Priority Data
Sep. 30, 2016 (GB) 1616707.4
Mar. 29, 2017 (GB) 1705056.8

(51) Int. Cl. C12P 19/02; C12P 19/12; C12P 7/06

(52) U.S. Cl. CPC C12P 19/02; C12P 7/06; C12P 19/12

(57) ABSTRACT

The present invention relates to a method of enzymatically degrading a polysaccharide, such as cellulose, comprising contacting the polysaccharide with one or more lytic polysaccharide monooxygenase (LPMO), in which the enzymatic degradation is carried out in the presence of at least one reducing agent, and hydrogen peroxide or a means which generates hydrogen peroxide in which the level of hydrogen peroxide is controlled to enhance and maintain the activity of the LPMO. The invention also extends to the additional use of hydrolytic enzymes such as hydrolases (e.g. cellulases, chitinases and/or ß-glucosidases) to increase the level or extent of degradation and to fermentation of the resulting sugars to generate an organic substance such as an alcohol, preferably ethanol, which may be used as a biofuel.

The present invention relates to a method of enzymatically degrading a polysaccharide, such as cellulose, comprising contacting the polysaccharide with one or more lytic polysaccharide monooxygenase (LPMO), in which the enzymatic degradation is carried out in the presence of at least one reducing agent, and hydrogen peroxide or a means which generates hydrogen peroxide in which the level of hydrogen peroxide is controlled to enhance and maintain the activity of the LPMO. The invention also extends to the additional use of hydrolytic enzymes such as hydrolases (e.g. cellulases, chitinases and/or ß-glucosidases) to increase the level or extent of degradation and to fermentation of the resulting sugars to generate an organic substance such as an alcohol, preferably ethanol, which may be used as a biofuel.

Depolymerization of biomass (e.g. cellulose) provides a useful source of saccharides such as glucose which may be used in fermentation reactions to generate biofuels such as ethanol or other platform chemicals.

The conversion of lignocellulosic feedstocks into ethanol has the advantages of the ready availability of large amounts of feedstock, avoiding burning or land filling the materials and the cleanness of the ethanol fuel. Wood, agricultural residues, herbaceous crops and municipal solid wastes have been considered as feedstocks for ethanol production. These materials primarily consist of cellulose, hemicellulose and the non-polysaccharide lignin. Once the cellulose is converted to glucose, the glucose is easily fermented by yeast into ethanol.

The depolymerisation of complex biomass, such as plant biomass primarily composed of cellulose, various hemicelluloses and lignin, relies on a network of enzymatic and chemical reactions that is still far from fully understood. Until recently, the degradation of the recalcitrant polysaccharides in plant biomass was thought to be achieved by an arsenal of hydrolytic enzymes called glycoside hydrolases (GHs) (Cragg et al., 2015, Curr. Opin. Chem. Biol., 29, 108-119). In some ecosystems, the enzymatic deconstruction process is supported by Fenton chemistry, i.e. redox metal-driven in situ generation of H₂O₂-derived hydroxyl radicals, the most powerful oxidizing species found on Earth (Gligorovski et al., 2015, Chem. Rev., 115, 13051-13092), which can attack both the polysaccharides and the lignin in plant biomass (Arantes and Goodell, “Current understanding of brown-rot fungal biodegradation mechanisms: a review” in Deterioration and protection of sustainable biomaterials (ACS Symposium series, 2014), vol. 1158, chap. 1, 3-21.).

In 2010, a new class of enzymes was discovered, which carry out oxidative cleavage of polysaccharides (Vaaje-Kolstad et al., 2010, Science., 330, 219-222). These enzymes, today known as lytic polysaccharide monooxygenases (LPMOs) (Horn et al., 2012, Biotechnol. Biofuels., 5, 45), are single-copper redox enzymes, that can activate molecular oxygen and hydroxylate the C1 or C4 positions of scissile glycosidic bonds (Vaaje-Kolstad et al., 2010, supra; Kjaergaard et al., 2014, Proc. Natl. Acad. Sci. U.S.A, 111, 8797-8802; Beeson et al., 2015, Annu. Rev. Biochem., 84, 923-946; and Walton and Davies, 2016, Curr. Opin. Chem. Biol., 31, 1-13.)

The inclusion of LPMOs in cellulolytic enzyme cocktails has had a major impact on industrial depolymerization of lignocellulosic biomass (Johansen, 2016, Biochem. Soc. Trans., 44, 143-149).

Despite their abundance in nature and their obvious industrial importance, the mode of action of LPMOs remains enigmatic, although some catalytic mechanisms have been proposed (Phillips et al., 2011, ACS Chem. Biol., 6, 1399-1406; Kjaergaard et al., 2014, supra; Walton and Davies, 2016, supra; Kim et al., 2014, Proc. Natl. Acad. Sci. U.S.A, 111, 149-154; and Frandsen et al., 2016, Nat. Chem. Biol., 12, 298-303).

The understanding at the time of the invention was that one LPMO reaction cycle requires the recruitment of two electrons (Vaaje-Kolstad et al., 2010, supra; Frandsen et al., 2016, supra; Beeson et al., 2012, J. Am. Chem. Soc., 134, 890-892). The first electron was often thought to be acquired via reduction of the LPMO's Cu(II) center to Cu(I) (Kracher et al., 2016, Science, 352, 1098-1101). When and how oxygen and the second electron are recruited remained an enigma. It appears impossible that an electron provider such as cellobiose dehydrogenase (Kracher et al., 2016, supra) carries out direct reduction of the active site copper while the LPMO is bound to the substrate, whereas it is unlikely that the protein unbinds during catalysis to allow such a direct second reduction step. The existence of an internal electron channel that would allow electron delivery to a substrate-bound enzyme has therefore been postulated (Walton and Davies, 2016, supra; Hemsworth et al., 2014, Nat. Chem. Biol., 10, 122-126; and Li et al., 2012, Structure, 20, 1051-61).

It appears increasingly clear that LPMO catalytic rates are dependent on the nature of the redox partner (Kracher et al., 2016, supra; Frommhagen et al., 2016, Biotechnol. Biofuels., 9, 1-17), despite the rate of LPMO reduction being considerably higher than reported rates for LPMO action.

Summarizing the above, prior to the present invention, the view on LPMO action was that they need one oxygen molecule, O₂, and two externally delivered electrons, delivered by what generally is referred to as a reductant, to complete a catalytic cycle.

In studies carried out using various reductants, referred to below as the Chl/light, Chl/light-AscA and AscA systems (wherein Chl is chlorophyllin and AscA is ascorbic acid) for LPMO activation and a bacterial C1-specific cellulose-active LPMO10 from Streptomyces coelicolor (ScLPMO10C) as the primary model enzyme it has now been found that H₂O₂, and not O₂, is the preferred co-substrate of LPMOs. This finding has major implications for the industrial application of LPMOs and for further optimization of these enzymes, e.g. by protein engineering, for industrial application.

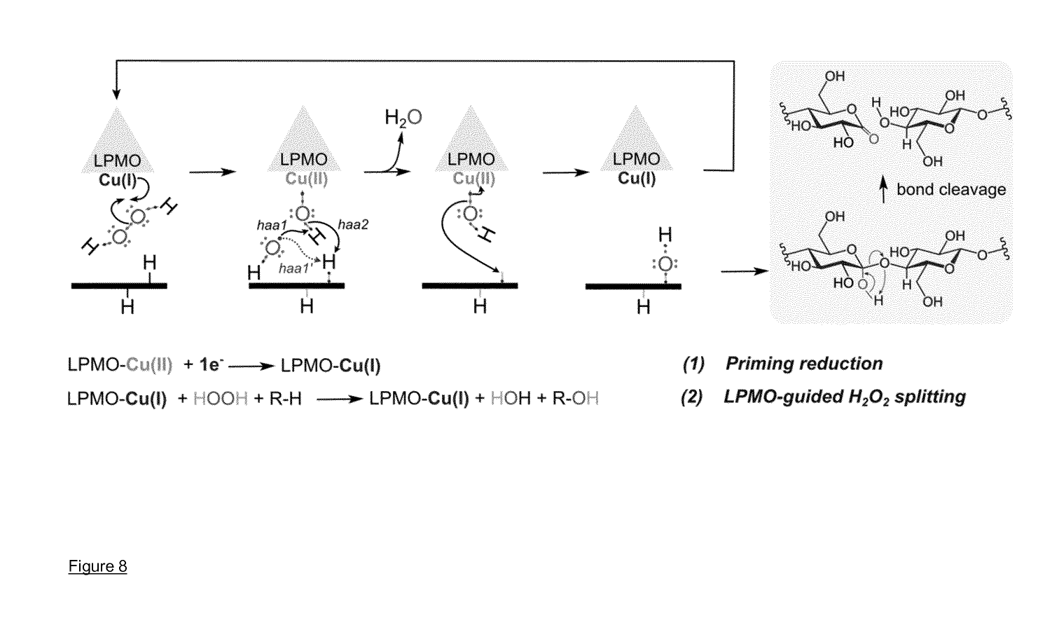

The results obtained are indicative of a catalytic mechanism in which an H₂O₂-derived oxygen atom, rather than an O₂-derived oxygen atom, would be introduced into the polysaccharide chain. Although not wishing to be bound by theory, in the proposed mechanism (FIG. 8), a priming reduction of the LPMO—Cu(II) to LPMO—Cu(I) occurs first. H₂O₂ then binds to the Cu(I) center and homolytic bond cleavage occurs which produces a hydroxyl radical. This leads to formation of a Cu(II)-bound hydroxide intermediate and substrate radical by one of several possible pathways. In each of these mechanisms, the reaction between a copper-hydroxide intermediate and the substrate radical leads to hydroxylation of the substrate and to regeneration of the Cu(I) center, which can enter a new catalytic cycle. The resulting hydroxylated polysaccharide is unstable and will undergo molecular rearrangement leading to bond cleavage and formation of an oxidized sugar (Beeson et al, 2012, supra).

As far as the inventors are aware, the biochemistry of the LPMO reaction, i.e. the splitting of H₂O₂ by a copper enzyme, is unprecedented in Nature (Mirica et al., 2004, Structure and Spectroscopy of Copper—Dioxygen Complexes., 104, 1013-104; Solomon et al., 2014, Chem. Rev., 114, 3659-3853). The findings reported herein explain several hitherto unexplained phenomena in LPMO biochemistry: (i) The consecutive delivery of two external electrons to the catalytic center is difficult to envisage, but with H₂O₂ being the co-substrate, recruitment of two electrons is not needed. (ii) The fact that most published catalytic rates for LPMOs are low and similar, and, most remarkably, independent of the LPMO or the substrate used (Vaaje-Kolstad et al., 2010, supra; Frandsen et al., 2016, supra; Agger et al., 2014, Proc. Natl. Acad. Sci., USA., 111, 6287-6292), is likely due to the fact that the rate-limiting factor in most experiments was H₂O₂ formation, but this was not recognized. (iii) The widely observed non-linearity of process kinetics during biomass degradation by enzyme cocktails or individual LPMOs is partly due to the self-inactivation of the LPMOs, both by over-stimulation and substrate-depletion. (iv) The increase in LPMO rate observed by Cannella et al. in their study on light-activation of LPMOs is due to production of hydrogen peroxide, not to the generation of some sort of “high energy electron” (Cannella et al., 2016, Nat. Comm., 7, doi:10.1038/ncomms11134).

The present findings have far reaching implications for biorefining processes. LPMOs are major players in commercial cellulose cocktails (Johansen, 2016, Biochem. Soc. Trans., 44, 143-149) but the presumed need for proper aeration and delivery of electrons at industrial scale pose challenges to process efficiency, as does the instability of LPMOs. It has now surprisingly been found that LPMO performance and stability can be controlled by controlling the supply of H₂O₂, a liquid, easy-to-handle, co-substrate. It has further been found that LPMOs can act in the presence of only catalytic amounts of reductant, which abolishes reductant-induced undesirable redox side reactions, and in the absence of molecular oxygen, abolishing the need for aeration.

In addition it has been found that hydrogen peroxide, at higher concentrations, inactivates LPMOs, especially if effective substrate concentrations (i.e. the amount of available productive binding sites for the LPMO) are low. Thus, whilst hydrogen peroxide has an activatory activity at lower levels, at higher levels it inactivates the enzyme and thus to optimize the reaction hydrogen peroxide must be kept within a narrow concentration range to allow use as a co-substrate but substantially avoid adverse effects on the enzyme.

Hydrogen peroxide has not previously been used as a co-substrate for LPMO. Hydrogen peroxide is well known as a pre-treatment of biomass prior to enzymatic treatment which serves to remove lignin which inhibits the later enzymatic process. The remaining hydrogen peroxide is quickly depleted and/or may be removed by washing or other separation processes (US2004231060, US2014004572, US2013189744, US2010159535, Correia et al., 2013, Bioresour Technol., 139, 249-56; Song et al., 2016, Bioresour Technol., 214, 30-6; Yu et al., 2015, Bioresour Technol., 187, 161-6; Jung et al., 2015, Bioresour Technol., 179, 467-72; Gao et al., 2014, Bioresour Technol., 171, 469-71; Cabrera et al., 2014, Bioresour Technol., 167, 1-7; Liu et al., 2014, Biotechnol Biofuels., 7(1), 48; Rabelo et al., 2008, Appl Biochem Biotechnol., 148(1-3), 45-58; Jabbour et al., 2013, Biotechnol Biofuels., 6(1), 58; and Rabelo et al., 2014, Fuel., 136, 349-357).

WO2016/096971 suggests the use of hydrogen peroxide in combination with a catalase to produce molecular oxygen for LPMOs in treating lignocellulosic material. Catalase has also been used to protect cellulolytic enzyme cocktails, which contain LPMOs, from inactivation caused indirectly by H₂O₂ (Scott et al., 2016, Biotechnol. Lett., 38, 425-434). It is well known that under certain conditions, H₂O₂ may react with, for example, free metal ions to generate reactive oxygen species that may damage enzymes such as cellulases. Furthermore, reactions that occur from enzymes or chemicals in the reaction may produce small amounts of hydrogen peroxide. However, the prior art does not identify that LPMOs use hydrogen peroxide as a co-substrate or that higher concentrations of hydrogen peroxide lead to inactivation of LPMOs or that the inactivation of LPMOs by hydrogen peroxide is specific for LPMOs (i.e. affects the LPMOs primarily due to a catalytic side reaction carried out by the LPMO). Thus, the prior art does not teach that use of hydrogen peroxide within a narrow concentration range would be particularly advantageous.

Thus, in a first aspect, the present invention provides a method of enzymatically degrading a polysaccharide comprising contacting said polysaccharide with one or more lytic polysaccharide monooxygenase (LPMO), wherein said enzymatic degradation is carried out in a reaction in the presence of:

a) at least one reducing agent; and

b) hydrogen peroxide or a means which generates hydrogen peroxide,

wherein the amount of hydrogen peroxide present during the degradation reaction is maintained in a concentration range at which the hydrogen peroxide acts as a co-substrate for said LPMO and said LPMO is inactivated by

(i) no more than 20% during a) the reaction time required to achieve 40% conversion of the polysaccharide or b) 4 hours of reaction time,

(ii) no more than 50% during a) the reaction time required to achieve 70% conversion of the polysaccharide or b) 12 hours of reaction time,

or

(iii) no more than 20% when said LPMO is contacted with said concentration of hydrogen peroxide in the presence of said polysaccharide and reducing agent for 20 minutes.

The essence of the invention is that LPMO action can be optimized by controlling the level of hydrogen peroxide in the reaction mixture. The level should be high enough to optimize LPMO action and low enough to prevent LPMO inactivation. As further explained hereinafter, the expert in the field of bioprocessing will recognize that the levels of hydrogen peroxide needed and acceptable degrees of LPMO inactivation during the course of a reaction will depend on the reaction conditions, in particular the type of substrate, the substrate concentration, the desired process time, the concentration of the LPMO and the presence and concentration of other enzymes such as cellulases or chitinases.

Preferably the concentration of hydrogen peroxide does not vary by more than 5, 10, 20 or 30% during the course of the reaction (e.g. during the reaction times defined herein).

Alternatively described, the invention provides a method of enzymatically degrading a polysaccharide comprising contacting said polysaccharide with one or more lytic polysaccharide monooxygenase (LPMO), wherein said enzymatic degradation is carried out in a reaction in the presence of:

a) at least one reducing agent; and

b) hydrogen peroxide or a means which generates hydrogen peroxide,

wherein

i) the amount of hydrogen peroxide present during the degradation reaction is maintained in a concentration range at which the hydrogen peroxide acts as a co-substrate for said LPMO; and/or

ii) the amount of hydrogen peroxide present during the degradation reaction is maintained in a concentration range at which the LPMO is not substantially inactivated; and/or

iii) the concentration of hydrogen peroxide does not vary by more than 5, 10, 20 or 30% during the course of the reaction; and/or

iv) the activity of said LPMO is controlled by adjusting the concentration of one or more components in the reaction to optimize production of oxidation products by the LPMO. In said reaction any one or more of (i) to (iv) may apply. The definitions and preferred embodiments of methods described herein (and uses of the method as described herein) apply similarly to the above alternatively described method. Thus, for example, methods of influencing the amount of hydrogen peroxide and adjusting the level of the components in the reaction to achieve the above aims, are as set out below.

As referred to herein “degrading” said polysaccharide refers to degradation by disruption of the glycosidic bonds connecting the sugar monomers in the polysaccharide polymer. This may also be referred to as depolymerization. In the present case the degradation occurs by oxidation resulting in the generation of an oxidized product, e.g. aldonic acid products when cellulose is degraded and the LMPO used has a preference for acting on C1.

The degradation of said polysaccharide is enhanced by the use of said reducing agents and hydrogen peroxide or means which generates the same relative to performance of said method without those means, thus the rate or degree of disruption of the glycosidic bonds that connect the sugar monomers is increased. This may readily be determined by measuring the product formation e.g. at certain defined time points or by measuring the amount of undegraded polysaccharide substrate which remains e.g. at certain defined time points. This can be carried out using methods that are well known in the art, based on e.g. determination of liberated reducing sugars (Horn, et al, 2004, Carbohydrate Polymers, 56 (1), 35-39 and references therein) or determination of liberated fragments, e.g. cellulose or chitin fragments, e.g. by quantitative analysis of chromatograms obtained upon High Performance Liquid Chromatography (Hoell et al, 2005, Biochim. Biophys. Acta, 1748(2), 180-190; Westereng et al., 2013, J. Chromatogr., 1271(1), 144-152). The generation of oxidized products as described in the Examples may also be used as an appropriate measure to assess the rate or degree of degradation. Said measure of degradation is assessed in the presence of one or more relevant hydrolytic enzymes which are preferably used as described hereinafter.

If the rate of degradation, i.e. the number of bonds disrupted in a certain time period is greater when the substrate has been exposed to the LPMO in the presence rather than absence of reducing agents and hydrogen peroxide or means to generate hydrogen peroxide, then the rate of degradation is considered to be enhanced. Preferably the use of reducing agents and hydrogen peroxide or means to generate hydrogen peroxide reduces the time taken for degradation (either complete or to the same level of partial degradation, e.g. when additional hydrolytic enzymes are used, see hereinafter) by at least 1.5, 2, 3, 4, 5, 6, 7, 8, 9 or 10 fold. Alternatively expressed, the use of reducing agents and hydrogen peroxide or means to generate hydrogen peroxide increases the rate of degradation by at least 1.5, 2, 3, 4, 5, 6, 7, 8, 9 or 10 fold. This enhanced degradative rate allows the use of reduced amounts of the other reactants, e.g. the concentration of the reducing agent and/or enzyme(s) used in the reaction may be reduced.

“Enzymatic” degradation refers to degradation that requires the catalytic activity of an enzyme, in this case at least LPMO. In preferred embodiments of the invention, as described hereinafter, additional enzymes are used in the method which contribute to the degradation (depolymerisation) of the polysaccharide.

Degradation of the polysaccharide may be partial or complete. In the case of complete degradation, complete saccharification is achieved, i.e. only soluble sugars (e.g. mono and di-saccharides) remain. In partial degradation, in addition to soluble sugars, larger oligosaccharides and polysaccharides remain. As described herein methods of the invention include methods in which only LPMOs are used for degradation or in which both LPMOs and hydrolytic or other enzymes are used for degradation. In the former case, preferably at least 0.05-10%, e.g. 0.05 to 5%, preferably 0.1 to 1% of the glycosidic bonds of the starting polysaccharide are degraded (i.e. disrupted by oxidation) into oligosaccharides which may be separate from the polysaccharide substrate or may remain associated despite cleavage. In the latter case in which hydrolytic enzymes are also used, preferably at least 30, 40 or 50% (especially preferably 60, 70, 80, 90, 95, 96, 97, 98, 99 or 100%) of the glycosidic bonds of the starting polysaccharide are degraded, i.e. cleaved. Alternatively expressed, in the latter case, preferably at least 50% (especially preferably 60, 70, 80, 90, 95, 96, 97, 98, 99 or 100%) of the starting polysaccharide is degraded into mono- or di-saccharides.

In relation to cellulose, the level of degradation may be assessed by determining the increase in the level of cellobiose and/or glucose (when LPMOs as well as hydrolytic enzymes are used).

As referred to herein “% conversion” defines the extent to which the glycosidic bonds in the polysaccharide have been cleaved. The percent conversion is defined relative to the maximum possible conversion under the conditions used (and before any inactivation of the LPMO that may occur) and may also be referred to as % of maximal (or achievable or relative) conversion.

It is well known in the art that, when using recalcitrant substrates such as cellulose or chitin, maximum achievable conversion by an LPMO acting alone normally implies that less than 10% of the glycosidic bonds in the substrate have been cleaved, even under optimal conditions. Complete saccharification of the substrate (i.e. cleavage of 100% of the glycosidic bonds), which is often industrially desirable, requires the use of additional enzymes, e.g. cellulases and beta-glucosidases. The interplay between the cellulases and the LPMO determines the efficiency of the overall saccharification process.

During the course of the reaction the number of glycosidic bonds which are cleaved in the substrate polysaccharide increases ultimately leading to a collection of mainly mono- or di-saccharides if the reaction is allowed to go to completion and both LPMOs and hydrolytic enzymes are used. Thus, during the course of the reaction the polysaccharide is converted to smaller oligosaccharides (including di- and tri-saccharides) and mono-saccharides. Often, industrial processes aim at conversion to mono-saccharides only.

When LPMOs alone are used, the conversion is limited. As noted above, only up to 10% of glycosidic bonds in the polysaccharide are cleaved using this enzyme in the reaction alone. In this case 100% conversion refers to the maximum possible conversion that could be achieved using the LPMO alone under the conditions in use. Thus, for example, if, under the conditions used, a maximum of 5% of the total glycosidic bonds could be cleaved, 50% conversion refers to cleavage of 2.5% of the glycosidic bonds of the polysaccharide.

When other enzymes are also used (at the same time), in the method, 100% conversion again refers to the maximum possible conversion that could be achieved using the enzymes under the conditions in use. If these enzymes could, together, achieve 50% cleavage of all glycosidic bonds in the polysaccharide (in some cases one of the final products is a di-saccharide, e.g. when cellulose is converted to cellobiose only, and 100% cleavage will therefore not occur), 50% conversion refers to cleavage of 25% of the glycoside bonds of the polysaccharide.

In the alternative, the extent of conversion may be defined in absolute terms, i.e. as a measure of the number of glycosidic bonds of the total present in the substrate which are cleaved. When this is intended reference is made to “absolute” (or total) conversion. In this case LMPO achieves, as noted above, a maximum of 10% absolute conversion whereas when hydrolytic enzymes are present a maximum of e.g. 50%-100% absolute conversion may be achieved, whereas complete saccharification (e.g. conversion of cellulose to glucose only) would equal an absolute conversion of 100%.

The yield of the products obtained may also be used to describe the efficacy of the reaction. In reactions in which LMPO is used alone, oxidized products are obtained as a result of the reaction. The yield of these products may be assessed as described in the Examples. In reactions in which hydrolytic enzymes are also used, the amounts of the resultant end products, e.g. di- or mono-saccharides may be assessed as an indication of efficacy.

The “reaction” is the chemical process in which the various components are brought into contact with one another and allowed to interact with one another for a certain period of time and under conditions to allow enzymatic degradation to occur. Conveniently the reaction and method of the invention may be conducted for at least 2 hours (e.g. at least 4, 8, 12, 16, 24, 48, 72 or more hours as described hereinbefore) or until at least 40% (preferably at least 50, 60, 70, 80 or 90%) (maximal or absolute) conversion of the polysaccharide has been achieved.

A “reaction mix” refers to the various components used in the method of the invention during the reaction which are present in a single medium to allow contact with one another. The reaction time refers to the time for which the reaction is conducted or a portion thereof.

As referred to herein said “polysaccharide” is a polymeric carbohydrate structure, formed of repeating units (preferably mono- or di-saccharides) joined together by glycosidic bonds and in the case of cellulose having the general formula (C₆H₁₀O₅)ₙ, e.g. in which 40≤n≤3000, or for chitin (C₈H₁₃O₅N)ₙ. The polysaccharide in the present invention is also referred to as the “substrate”. Preferably said polysaccharide is at least partially crystalline, i.e. is in a crystalline form or has crystalline portions, i.e. a form or portion which shows a repeating, three-dimensional pattern of atoms, ions or molecules having fixed distances between the constituent parts.

Preferably said polysaccharide is cellulose, hemicellulose or chitin and may be in isolated form or may be present in impure form, e.g. in a cellulose-, hemicellulose- or chitin-containing material (i.e. a polysaccharide-containing material), which optionally may contain other polysaccharides, e.g. in the case of cellulose, hemicellulose and/or pectin may also be present. The polysaccharide, which may contain cellulose, hemicellulose or chitin, for example, may be a biomass which is derived or obtained from biological material.

By way of example, the cellulose-containing material may be stems, leaves, hulls, husks and cobs of plants or leaves, branches and wood of trees. The cellulose-containing material can be, but is not limited to, herbaceous material, agricultural residues, forestry residues, municipal solid wastes, waste paper and pulp and paper mill residues. The cellulose-containing material can be any type of biomass including, but not limited to, wood resources, municipal solid waste, wastepaper, crops and crop residues (see, for example, Wiselogel et al., 1995, in “Handbook on Bioethanol” (Charles E. Wyman, editor), pp. 105-118). Preferably the cellulose-containing material is in the form of lignocellulose, e.g. a plant cell wall material containing lignin, cellulose and hemicellulose in a mixed matrix.

In a preferred aspect, the cellulose-containing material is corn stover. In another preferred aspect, the cellulose-containing material is corn fiber, corn cobs, switch grass or rice straw. In another preferred aspect, the cellulose-containing material is paper and pulp processing waste. In another preferred aspect, the cellulose-containing material is woody or herbaceous plants. In another preferred aspect, the cellulose-containing material is bagasse. Other preferred materials include Industrially relevant biomasses such as sulfite-pulped Norway spruce or steam exploded birch.

“Cellulose” is a polymer of the simple sugar glucose covalently bonded by ß-1, 4-linkages. Cellulose is a straight chain polymer: unlike starch, no coiling or branching occurs and the molecule adopts an extended and rather stiff rod-like conformation, aided by the equatorial conformation of the glucose residues. The multiple hydroxyl groups on the glucose from one chain form hydrogen bonds with oxygen molecules on the same or on a neighbour chain, holding the chains firmly together side-by-side and forming microfibrils with high tensile strength.

Compared to starch, cellulose is also much more crystalline. Whereas starch undergoes a crystalline to amorphous transition when heated beyond 60-70° C. in water (as in cooking), cellulose requires a temperature of 320° C. and pressure of 25 MPa to become amorphous in water.

Several different crystalline structures of cellulose are known, corresponding to the location of hydrogen bonds between and within strands. Natural cellulose is cellulose I, with structures I_{α} and I_{β}. Cellulose produced by bacteria and algae is enriched in I_{α} while cellulose of higher plants consists mainly of I_{β}. Cellulose in regenerated cellulose fibers is cellulose II. The conversion of cellulose I to cellulose II is not reversible, suggesting that cellulose I is metastable and cellulose II is stable. With various chemical treatments it is possible to produce the structures cellulose III and cellulose IV.

The term “hemicellulose” refers to a collection of different polysaccharides containing several sugars in addition to glucose, especially xylose but also including mannose, galactose, rhamnose and arabinose. Hemicellulose consists of shorter chains than cellulose; around 200 sugar units. Furthermore, hemicellulose is branched, whereas cellulose is unbranched. Known hemicellulose types include xyloglucan, glucomannan, galactoglucomann, mannan, xylan and arabinoxylan.

“Chitin” is defined herein as any polymer containing β(1-4) linked N-acetylglucosamine residues that are linked in a linear fashion. Crystalline chitin in the α form (where the chains run anti-parallel), β form (where the chains run parallel) or γ form (where there is a mixture of parallel and antiparallel chains), amorphous chitin, colloidal chitin, chitin forms in which part (e.g. up to 5, 10, 15 or 20%) of the N-acetylglucosamine sugars are deacetylated are all included within the definition of this term.

Other forms of chitin that are found in nature include copolymers with proteins and these copolymers, which include protein chitin matrices that are found in insect and crustacean shells and any other naturally occurring or synthetic copolymers comprising chitin molecules as defined herein, are also included within the definition of “chitin”.

The term “chitin” thus includes purified crystalline α, β and γ preparations, or chitin obtained or prepared from natural sources, or chitin that is present in natural sources. Examples of such natural sources include squid pen, crustaceans shells (e.g. shrimp or crab shells), insect cuticles and fungal mycelia and cell walls. Chitin may be sourced from insect-derived biomass or waste from production facilities for fungi, for example. Examples of commercially available chitins are those available from sources such as France Chitin, Hov-Bio, Sigma, Sekagaku Corp, amongst others.

As referred to herein “contacting” said polysaccharide with an LPMO refers to bringing the two entities together in an appropriate manner to allow the catalytic properties of the enzyme to be effective.

The precise kinetics of the reaction between the LPMO and the polysaccharide will depend on many factors, such as the type of polysaccharide to be degraded, the purity of the polysaccharide, the amount of enzyme present, the temperature, the pH, the mixing mode, the presence of reductant and, as disclosed herein, the presence of H₂O₂. The type of polysaccharide and its degree of amorphousness will vary with the substrate source and isolation/purification process, but can be assessed, for example, by measuring the degree of crystallinity of the substrate (which is a method known in the art) and/or the chemical composition of the substrate.

Taking these considerations into account one can determine appropriate incubation times and conditions to maximize degradation (e.g. hydrolysis with glycoside hydrolases). Exemplary methods are discussed below.

Thus, the polysaccharide and LPMO are mixed together or contacted with one another to allow their interaction. This may simply involve directly mixing solutions of the different components or applying the enzyme to the polysaccharide-containing material as described hereinafter. As described hereinafter, preferably additional enzymes are used in the reaction whose nature and concentration may be selected appropriately depending on the substrate to be used and other reaction conditions.

As referred to herein “one or more” (or “at least one”) preferably denotes 2, 3, 4, 5 or 6 or more of the recited entities, e.g. enzymes, reducing agents or components. In the case of enzymes, when more than one of the enzymes is used they may be selected in line with the substrate to be used, e.g. to provide complementary or synergistic action. Thus, for example, LPMOs may be combined which are effective on different regions of the substrate, e.g. different crystal faces. Preferred combinations are described hereinafter.

As used herein a “lytic polysaccharide monooxygenase” is an enzyme which, as discussed above, uses hydrogen peroxide as a co-substrate for cleavage of glycosidic bonds in polysaccharides, preferably cellulose or chitin. For LPMOs that act on the non-reducing side of the glycosidic bond (C1 in the case of cellulose), the newly generated chain ends are one normal non-reducing end and an oxidized “acidic” end that, in the case of chitin is a 2-(Acetylamino)-2-deoxy-D-gluconic acid and in the case of cellulose is a D-gluconic acid (aldonic acid). For LPMOs that act on the reducing side of the glycosidic bond (C4 in the case of cellulose), the newly generated chain ends are one normal reducing end and an oxidized non-reducing end that is a 4-ketosugar (Isaksen et al., 2014, J. Biol. Chem., 289(5), 2632-2642). Notably, some cellulose-active LPMOs only act on C1, some only act on C4, whereas some show mixed activity, acting both on C1 and C4, yielding both types of the oxidized products mentioned above.

LPMOs have a metal binding site and require the presence of a divalent metal ion (copper) for full activity. Preferred LPMOs include those from the Auxiliary Activity (AA) family 9, 10, 11 or 13 (also known as LPMO9, LPMO10, LPMO11 and LPMO13, respectively). (AA10 and AA9 were previously referred to as CBM33 and GH61 enzymes, respectively.) The metal is bound by at least three ligands that are fully conserved in both families: (1) a histidine that is in position 1 of the mature protein (i.e. the N-terminal residue of the protein after the signal peptide for secretion has been cleaved off); (2) the N-terminal amino group of the mature protein; (3) another histidine residue that is fully conserved within LPMO families.

LPMOs belonging to the AA9, AA10, AA11 and AA13 families can be identified by analysis of gene sequences (and the corresponding predicted amino acid sequences of the gene products), using standard bioinformatic methods (Levasseur et al., 2013, Biotechnol. Biofuels., 6(1), 41. For example one can use an existing multiple sequence alignment of AA9, AA10, AA11 or AA13 enzymes, for example represented by a Hidden Markov Model, to search for homologous sequences in sequence databases. Sequences retrieved by such searches would be highly likely to be active LPMOs. More certainty may be obtained by (1) checking that the gene encodes a protein with a signal peptide for secretion, using e.g. the programme SignalP; (2) checking that the N-terminal residue after cleavage of the signal peptide (cleavage site to be predicted using e.g. SignalP) is a histidine; (3) checking that there is another histidine in the protein sequence that aligns with a fully or almost fully (>90%) conserved histidine in the multiple sequence alignment; (4) using model-building by homology, using automated servers such as Swiss-Model, to check that this second histidine is likely to be located close to the N-terminus and the N-terminal histidine.

The skilled person can readily determine by experiment whether a protein is an LPMO according to the above described definition by determining if it can cleave glycosidic bonds by oxidation and if this process becomes more effective in the presence of hydrogen peroxide (at appropriate levels as described herein) and a reductant. Experiments such as those conducted in the examples may be used, thus the effect of reductants and hydrogen peroxide on enzymatic activity may be assessed. Even, without using H₂O₂, LPMO activity, likely at suboptimal levels, is readily demonstrated by only using a known reductant such as ascorbic acid and by using mass spectrometry or HPLC for product detection (Vaaje-Kolstad et al., 2010, supra; Agger et al., 2014, supra)

Preferably said LPMO contains at least one domain that on the basis of sequence similarity as analyzed in e.g. the current CAZy database (www.cazy.org, Davies & Henrissat, 2002, Biochem Soc T 30, 291-297 and Bourne & Henrissat, 2001, supra) is classified as a AA9, AA10, AA11 and AA13 family protein. Some LPMOs act on cellulose, some act on chitin, some act on both, and yet other LPMOs act on other substrates. Known other substrates currently include xyloglucan, xylan, starch, glucomannan and certain beta-glucan and it is fully expected that additional polysaccharide substrates will be identified. When the AA9, AA10, AA11 and AA13 containing proteins have more than one domain, the additional domains are usually coupled to the C-terminus of the AA9, AA10, AA11 and AA13 domain because the N-terminus of the AA9, AA10, AA11 and AA13 domain is essential for LPMO activity.

The LPMO is preferably an AA9 or AA10 enzyme.

The LPMO used in methods of the invention may contain, consist or consist essentially of an AA9, AA10, AA11 or AA13 domain or protein or a biologically active fragment thereof. In this context, “consists essentially of” indicates that additional amino acids may be present in the protein, in addition to those that make up the AA9, AA10, AA11 or AA13 domain or protein. Preferably, when such additional amino acids are present, there are 1-3, 1-5, 1-10, 10-20, 20-30, 30-40, 40-50, 50-60, 60-70, 70-80, 80-90 or 90-100 or more, even up to 500 or 1000, additional amino acids present. These additional amino acids are in general present C-terminal to the AA9, AA10, AA11 or AA13 domain.

As mentioned above, the LPMO can comprise a AA9, AA10, AA11 or AA13 domain or protein. Additional modules or domains may thus be present in the protein, which, when present are preferably at the C-terminus.

In a preferred feature a native AA9, AA10, AA11 or AA13 domain or protein or a biologically active fragment thereof is used though variants of the native form may be used, some of which are described hereinafter.

LPMOs which comprise or consist of a AA9, AA10, AA11 or AA13 domain or protein or its fragments or variants are referred to herein, collectively, as AA9, AA10, AA11 or AA13 proteins or AA9, AA10, AA11 or AA13 family members or proteins.

Examples of suitable native proteins in the AA9 family are provided in Table 1 below which provides relevant database accession numbers which are hereby incorporated by reference. Other appropriate enzymes may readily identified in the CAZy database or by sequence comparison to known enzymes.

TABLE 1
AA9 enzymes

| Protein Name | Organism | GenBank | Uniprot |
|---|---|---|---|
| Cel1 | Agaricus bisporus D649 | AAA53434.1 | Q00023 |
| AfA5C5.025 | Aspergillus fumigatus | CAF31975.1 | Q6MYM8 |
| endo-β-1,4- | Aspergillus kawachii | BAB62318.1 | Q96WQ9 |
| glucanase | | | |
| B (EglB) (Cel61A) | | | |
| AN1041.2 | Aspergillus nidulans | EAA65609.1 | C8VTW9 |
| | FGSC A4 | | |
| AN3511.2 | Aspergillus nidulans | EAA59072.1 | |
| | FGSC A4 | | |
| AN9524.2 | Aspergillus nidulans | CBF83171.1 | C8VI93 |
| | FGSC A4 | EAA66740.1 | |
| AN7891.2 | Aspergillus nidulans | EAA59545.1 | |
| | FGSC A4 | | |
| AN6428.2 | Aspergillus nidulans | EAA58450.1 | C8V0F9 |
| | FGSC A4 | | |
| AN3046.2 | Aspergillus nidulans | EAA63617.1 | C8VIS7 |
| | FGSC A4 | | |
| AN3860.2 | Aspergillus nidulans | EAA59125.1 | C8V6H2 |
| | FGSC A4 | | |
| endo-β-1,4- | Aspergillus nidulans | ABF50850.1 | |
| glucanase | FGSC A4 | EAA64722.1 | |
| (AN1602.2) | | | |
| AN2388.2 | Aspergillus nidulans | EAA64499.1 | C8VNP4 |
| | FGSC A4 | | |
| An14g02670 | Aspergillus niger | CAK46515.1 | A2R313 |
| | CBS 513.88 | | |
| An15g04570 | Aspergillus niger | CAK97324.1 | A2R5J9 |
| | CBS 513.88 | | |
| An15g04900 | Aspergillus niger | CAK42466.1 | A2R5N0 |
| | CBS 513.88 | | |
| An04g08550 | Aspergillus niger | CAK38942.1 | A2QJX0 |
| | CBS 513.88 | | |
| An08g05230 | Aspergillus niger | CAK45495.1 | A2QR94 |
| | CBS 513.88 | | |
| An12g02540 | Aspergillus niger | CAK41095.1 | A2QYU6 |
| | CBS 513.88 | | |
| An12g04610 | Aspergillus niger | CAK97151.1 | A2QZE1 |
| | CBS 513.88 | | |
| AO090005000531 | Aspergillus oryzae RIB40 | BAE55582.1 | |
| AO090001000221 | Aspergillus oryzae RIB40 | BAE56764.1 | |
| AO090138000004 | Aspergillus oryzae RIB40 | BAE64395.1 | |
| AO090023000056 | Aspergillus oryzae RIB40 | BAE58643.1 | |
| AO090023000159 | Aspergillus oryzae RIB40 | BAE58735.1 | |
| AO090023000787 | Aspergillus oryzae RIB40 | BAE59290.1 | |
| AO090103000087 | Aspergillus oryzae RIB40 | BAE65561.1 | |
| AO090012000090 | Aspergillus oryzae RIB40 | BAE60320.1 | |
| GH61A | Botryosphaeria rhodina | CAJ81215.1 | |
| | CBS 247.96 | | |
| GH61B | Botryosphaeria rhodina | CAJ81216.1 | |
| | CBS 247.96 | | |
| GH61C | Botryosphaeria rhodina | CAJ81217.1 | |
| | CBS 247.96 | | |
| GH61D | Botryosphaeria rhodina | CAJ81218.1 | |
| | CBS 247.96 | | |
| Cel6 | Cochliobolus | AAM76663.1 | Q8J0H7 |
| | heterostrophus C4 | | |
| unnamed protein | Coprinopsis cinerea | CAG27578.1 | |
| product | | | |
| Cel1 | Cryptococcus neoformans | AAC39449.1 | O59899 |
| | var. neoformans | | |
| CNA05840 (Cel1) | Cryptococcus neoformans | AAW41121.1 | |
| | var. neoformans JEC21 | | |
| xylanase II | Fusarium oxysporum F3 | | |
| (peptide fragment) | | | |
| Sequence 122805 | Gibberella zeae | ABT35335.1 | |
| from U.S. Pat. No. | | | |
| 7,214,786 | | | |
| FG03695.1 | Gibberella zeae PH-1 | XP_383871.1 | |
| (Cel61E) | | | |
| ORF (possible | Glomerella graminicola | CAQ16278.1 | B5WYD8 |
| fragment) | M2 | | |
| ORF | Glomerella graminicola | CAQ16206.1 | B5WY66 |
| | M2 | | |
| ORF | Glomerella graminicola | CAQ16208.1 | B5WY68 |
| | M2 | | |
| ORF | Glomerella graminicola | CAQ16217.1 | B5WY77 |
| | M2 | | |
| unnamed protein | Humicola insolens | CAG27577.1 | |
| product | | | |
| cellulase- | Hypocrea jecorina QM6A | AAP57753.1 | Q7Z9M7 |
| enhancing | | ABH82048.1 | |
| factor (Cel61B) | | ACK19226.1 | |
| | | ACR92640.1 | |
| endo-β-1,4- | Hypocrea jecorina | CAA71999.1 | O14405.1 |
| glucanase | RUTC-30 | | |
| IV (EGIV; Egl4) | | | |
| (Cel61A) | | | |
| MG05364.4 | Magnaporthe grisea 70-15 | EAA54572.1 | |
| | | XP_359989.1 | |
| MG07686.4 | Magnaporthe grisea 70-15 | EAA53409.1 | |
| | | XP_367775.1 | |
| MG07300.4 | Magnaporthe grisea 70-15 | EAA56945.1 | |
| | | XP_367375.1 | |
| MG07575.4 | Magnaporthe grisea 70-15 | EAA53298.1 | |
| | | XP_367664.1 | |
| MG08020.4 | Magnaporthe grisea 70-15 | EAA57051.1 | |
| | | XP_362437.1 | |
| MG02502.4 | Magnaporthe grisea 70-15 | EAA54517.1 | |
| | | XP_365800.1 | |
| MG08254.4 | Magnaporthe grisea 70-15 | EAA57285.1 | |
| | | XP_362794.1 | |
| MG08066.4 | Magnaporthe grisea 70-15 | EAA57097.1 | |
| | | XP_362483.1 | |
| MG04547.4 | Magnaporthe grisea 70-15 | EAA50788.1 | |
| | | XP_362102.1 | |
| MG08409.4 | Magnaporthe grisea 70-15 | EAA57439.1 | |
| | | XP_362640.1 | |
| MG09709.4 | Magnaporthe grisea 70-15 | EAA49718.1 | |
| | | XP_364864.1 | |
| MG04057.4 | Magnaporthe grisea 70-15 | EAA50298.1 | |
| | | XP_361583.1 | |
| MG06069.4 | Magnaporthe grisea 70-15 | EAA52941.1 | |
| | | XP_369395.1 | |
| MG09439.4 | Magnaporthe grisea 70-15 | EAA51422.1 | |
| | | XP_364487.1 | |
| MG06229.4 | Magnaporthe grisea 70-15 | EAA56258.1 | |
| | | XP_369714.1 | |
| MG07631.4 | Magnaporthe grisea 70-15 | EAA53354.1 | |
| | | XP_367720.1 | |
| MG06621.4 | Magnaporthe grisea 70-15 | XP_370106.1 | |
| (fragment) | | | |
| NCU07898.1 | Neurospora crassa | EAA33178.1 | |
| | OR74A | XP_328604.1 | |
| NCU05969.1 | Neurospora crassa | EAA29347.1 | |
| | OR74A | XP_325824.1 | |
| NCU02916.1 | Neurospora crassa | EAA36362.1 | |
| | OR74A | XP_330104.1 | |
| NCU03000.1 | Neurospora crassa | CAB97283.2 | Q9P3R7 |
| (B24P7.180) | OR74A | EAA36150.1 | |
| | | XP_330187.1 | |
| NCU07760.1 | Neurospora crassa | EAA29018.1 | |
| | OR74A | XP_328466.1 | |
| NCU07520.1 | Neurospora crassa | EAA29132.1 | |
| | OR74A | XP_327806.1 | |
| NCU01050.1 | Neurospora crassa | CAD21296.1 | Q8WZQ2 |
| (G15G9.090) | OR74A | EAA32426.1 | |
| | | XP_326543.1 | |
| NCU02240.1 | Neurospora crassa | EAA30263.1 | |
| | OR74A | XP_331016.1 | |
| NCU02344.1 | Neurospora crassa | CAF05857.1 | |
| (B23N11.050) | OR74A | EAA30230.1 | |
| | | XP_331120.1 | |
| NCU00836.1 | Neurospora crassa | EAA34466.1 | |
| | OR74A | XP_325016.1 | |
| NCU08760.1 | Neurospora crassa | EAA26873.1 | |
| | OR74A | XP_330877.1 | |
| NCU07974.1 | Neurospora crassa | EAA33408.1 | |
| | OR74A | XP_328680.1 | |
| NCU03328.1 | Neurospora crassa | CAD70347.1 | Q873G1 |
| (B10C3.010) | OR74A | EAA26656.1 | |
| | | XP_322586.1 | |
| NCU01867.1 | Neurospora crassa | CAE81966.1 | Q7SHD9 |
| (B13N4.070) | OR74A | EAA36262.1 | |
| | | XP_329057.1 | |
| β-1,3-1,4- | Paecilomyces | | |
| glucanase | thermophila J18 | | |
| (peptide fragment) | | | |
| Pc20g11100 | Penicillium chrysogenum | CAP86439.1 | B6HG02 |
| | Wisconsin 54-1255 | | |
| Pc12g13610 | Penicillium chrysogenum | CAP80988.1 | B6H016 |
| | Wisconsin 54-1255 | | |
| Pc13g07400 | Penicillium chrysogenum | CAP91809.1 | B6H3U0 |
| | Wisconsin 54-1255 | | |
| Pc13g13110 | Penicillium chrysogenum | CAP92380.1 | B6H3A3 |
| | Wisconsin 54-1255 | | |
| Cel61 (Cel61A) | Phanerochaete | AAM22493.1 | Q8NJI9 |
| | chrysosporium | | |
| | BKM-F-1767 | | |
| Pa_4_1020 | Podospora anserina | CAP61476.1 | B2ADG1 |
| | S mat+ | | |
| unnamed protein | Podospora anserina | CAP68173.1 | B2AUV0 |
| product | S mat+ | | |
| unnamed protein | Podospora anserina | CAP68309.1 | B2AV86 |
| product | S mat+ | | |
| unnamed protein | Podospora anserina | CAP61650.1 | B2ADY5 |
| product | S mat+ | | |
| unnamed protein | Podospora anserina | CAP68352.1 | B2AVC8 |
| product | S mat+ | | |
| Pa_7_3160 | Podospora anserina | CAP68375.1 | B2AVF1 |
| | S mat+ | | |
| unnamed protein | Podospora anserina | CAP71532.1 | B2B346 |
| product | S mat+ | | |
| (fragment) | | | |
| unnamed protein | Podospora anserina | CAP71839.1 | B2B403 |
| product | S mat+ | | |
| unnamed protein | Podospora anserina | CAP72740.1 | B2B4L5 |
| product | S mat+ | | |
| Pa_5_8940 | Podospora anserina | CAP64619.1 | B2AKU6 |
| | S mat+ | | |
| unnamed protein | Podospora anserina | CAP73072.1 | B2B5J7 |
| product | S mat+ | | |
| unnamed protein | Podospora anserina | CAP64732.1 | B2AL94 |
| product | S mat+ | | |
| Pa_2_6530 | Podospora anserina | CAP73254.1 | B2B629 |
| | S mat+ | | |
| unnamed protein | Podospora anserina | CAP73311.1 | B2B686 |
| product | S mat+ | | |
| unnamed protein | Podospora anserina | CAP73320.1 | B2B695 |
| product | S mat+ | | |
| unnamed protein | Podospora anserina | CAP64865.1 | B2ALM7 |
| product | S mat+ | | |
| unnamed protein | Podospora anserina | CAP65111.1 | B2AMI8 |
| product | S mat+ | | |
| unnamed protein | Podospora anserina | CAP65855.1 | B2APD8 |
| product | S mat+ | | |
| unnamed protein | Podospora anserina | CAP65866.1 | B2APE9 |
| product | S mat+ | | |
| unnamed protein | Podospora anserina | CAP65971.1 | B2API9 |
| product | S mat+ | | |
| unnamed protein | Podospora anserina | CAP66744.1 | B2ARG6 |
| product | S mat+ | | |
| Pa_1_500 | Podospora anserina | CAP59702.1 | B2A9F5 |
| | S mat+ | | |
| unnamed protein | Podospora anserina | CAP61048.1 | B2AC83 |
| product | S mat+ | | |
| unnamed protein | Podospora anserina | CAP67176.1 | B2AS05 |
| product | S mat+ | | |
| Pa_1_22040 | Podospora anserina | CAP67190.1 | B2AS19 |
| | S mat+ | | |
| unnamed protein | Podospora anserina | CAP67201.1 | B2AS30 |
| product | S mat+ | | |
| unnamed protein | Podospora anserina | CAP67466.1 | B2ASU3 |
| product | S mat+ | | |
| unnamed protein | Podospora anserina | CAP67481.1 | B2ASV8 |
| product | S mat+ | | |
| unnamed protein | Podospora anserina | CAP67493.1 | B2ASX0 |
| product | S mat+ | | |
| unnamed protein | Podospora anserina | CAP70156.1 | B2AZV6 |
| product | S mat+ | | |
| Pa_4_350 | Podospora anserina | CAP61395.1 | B2AD80 |
| | S mat+ | | |
| Pa_1_16300 | Podospora anserina | CAP67740.1 | B2ATL7 |
| | S mat+ | | |
| unnamed protein | Podospora anserina | CAP70248.1 | B2AZD4 |
| product | S mat+ | | |
| SMU2916 | Sordaria macrospora | CAQ58424.1 | |
| (fragment) | k-hell | | |
| cellulase- | Thermoascus aurantiacus | ABW56451.1 | |
| enhancing factor | | ACS05720.1 | |
| (GH61A) | | | |
| unnamed protein | Thielavia terrestris | CAG27576.1 | |
| product | | | |
| cellulase- | Thielavia terrestris | ACE10231.1 | |
| enhancing factor | NRRL 8126 | | |
| (GH61B) | | | |
| Sequence 4 from | Thielavia terrestris | ACE10232.1 | |
| U.S. Pat. No. | NRRL 8126 | | |
| 7,361,495 | | | |
| (GH61C) | | | |
| Sequence 4 from | Thielavia terrestris | ACE10232.1 | |
| U.S. Pat. No. | NRRL 8126 | | |
| 7,361,495 | | | |
| (GH61C) | | | |
| Sequence 6 from | Thielavia terrestris | ACE10233.1 | |
| U.S. Pat. No. | NRRL 8126 | | |
| 7,361,495 | | | |
| (GH61D) | | | |
| Sequence 6 from | Thielavia terrestris | ACE10233.1 | |
| U.S. Pat. No. | NRRL 8126 | | |
| 7,361,495 | | | |
| (GH61D) | | | |
| cellulase- | Thielavia terrestris | ACE10234.1 | |
| enhancing factor | NRRL 8126 | | |
| (131562) | | | |
| (GH61E) | | | |
| Sequence 10 from | Thielavia terrestris | ACE10235.1 | |
| U.S. Pat. No. | NRRL 8126 | | |
| 7,361,495 | | | |
| (GH61G) | | | |
| Sequence 10 from | Thielavia terrestris | ACE10235.1 | |
| U.S. Pat. No. | NRRL 8126 | | |
| 7,361,495 | | | |
| (GH61G) | | | |
| endoglucanase | Trichoderma | ADB89217.1 | |
| (EnGluIV; | saturnisporum | | |
| EndoGluIV) | | | |
| Endoglucanase IV | Trichoderma sp. SSL | ACH92573.1 | B5TYI4 |
| endoglucanase IV | Trichoderma viride | ACD36973.1 | B4YEW3 |
| (EgiV) | AS 3.3711 | | |
| endoglucanase VII | Trichoderma viride | ACD36971.1 | B4YEW1 |
| (EgvII) | AS 3.3711 | | |
| Endoglucanase II | Volvariella volvacea | AAT64005.1 | Q6E5B4 |
| (EgII) | | | |
| unknown | Zea mays B73 | ACF78974.1 | B4FA31 |
| (ZM_ | | ACR36748.1 | |
| BFc0036G02) | | | |

Examples of known AA10 proteins which may be used in methods of the invention and relevant database accession numbers (which are hereby incorporated by reference) are set out in Table 2:

TABLE 2
AA10 enzymes

| | | |
|---|---|---|
| PROTEIN | ORGANISM | GENBANK/GENPEPT |

BACTERIA

| | | | |
|---|---|---|---|
| Cbp1 | Alteromonas sp. O-7 | AB063629 | BAB79619.1 |
| chitin binding protein ChbA | Bacillus amyloliquefaciens ALKO 2718 | AF181997 | AAG09957.1 |
| BA_3348 | Bacillus anthracis str. A2012 | NC_003995 | NP_656708.1 |
| BA2827 | Bacillus anthracis str. Ames | AE017033 | AAP26659.1 |
| | | NC_003997 | NP_845173.1 |
| BA2793 | Bacillus anthracis str. Ames | AE017032 | AAP26628.1 |
| | | NC_003997 | NP_845142.1 |
| GBAA2827 | Bacillus anthracis str. Ames 0581 | AE017334 | AAT31944.1 |
| GBAA2793 | Bacillus anthracis str. Ames 0581 | AE017334 | AAT31910.1 |
| BAS2636 | Bacillus anthracis str. Sterne | AE017225 | AAT54946.1 |
| BAS2604 | Bacillus anthracis str. Sterne | AE017225 | AAT54914.1 |
| BCE2855 | Bacillus cereus ATCC 10987 | AE017273 | AAS41767.1 |
| | | NC_003909 | NP_979159.1 |
| BCE2824 | Bacillus cereus ATCC 10987 | AE017273 | AAS41736.1 |
| | | NC_003909 | NP_979128.1 |
| BC2827 | Bacillus cereus ATCC 14579 | AE017007 | AAP09778.1 |
| | | NC_004722 | NP_832577.1 |
| BC2798 | Bacillus cereus ATCC 14579 | AE017007 | AAP09751.1 |
| | | NC_004722 | NP_832550.1 |
| pE33L466_0276 (ChbA) | Bacillus cereus E33L | CP000040 | AAY60428.1 |
| BTZK2523 (ChB) | Bacillus cereus ZK | CP000001 | AAU17736.1 |
| BTZK2552 (ChB) | Bacillus cereus ZK | CP000001 | AAU17707.1 |
| ABC1161 | Bacillus clausii KSM-K16 | AP006627 | BAD63699.1 |
| BH1303 | Bacillus halodurans C-125 | AP001511 | BAB05022.1 |
| | | NC_002570 | NP_242169.1 |
| BLi00521 or BL00145 | Bacillus licheniformis DSM 13 ATCC 14580 | CP000002 | AAU22121.1 |
| | | AE017333 | AAU39477.1 |
| BT9727_2586 (ChB) | Bacillus thuringiensis serovar konkukian str. 97-27 | AE017355 | AAT61310.1 |
| BT9727_2556 (ChB) | Bacillus thuringiensis serovar konkukian str. 97-27 | AE017355 | AAT61323.1 |
| BMAA1785 | Burkholderia mallei ATCC 23344 | CP000011 | AAU45854.1 |
| BMA2896 | Burkholderia mallei ATCC 23344 | CP000010 | AAU48386.1 |
| BURPS1710b_0114 | Burkholderia pseudomallei 1710b | CP000124 | ABA49030.1 |
| BURPS1710b_A2047 | Burkholderia pseudomallei 1710b | CP000125 | ABA53645.1 |
| BPSL3340 | Burkholderia pseudomallei K96243 | BX571965 | CAH37353.1 |
| BPSS0493 | Burkholderia pseudomallei K96243 | BX571966 | CAH37950.1 |
| Bcep18194_C6726 | Burkholderia sp. 383 | CP000150 | ABB05775.1 |
| BTH_II1925 | Burkholderia thailandensis E264; ATCC 700388 | CP000085 | ABC34637.1 |
| BTH_I3219 | Burkholderia thailandensis E264; ATCC 700388 | CP000086 | ABC38514.1 |
| -1,4-mannanase (ManA) | Caldibacillus cellulovorans | AF163837 | AAF22274.1 |
| CV0554 | Chromobacterium violaceum ATCC 12472 | AE016911 | AAQ58230.1 |
| | | NC_005085 | NP_900224.1 |
| CV0553 | Chromobacterium violaceum ATCC 12472 | AE016911 | AAQ58229.1 |
| | | NC_005085 | NP_900223.1 |
| CV2592 (CpbD) | Chromobacterium violaceum ATCC 12472 | AE016919 | AAQ60262.1 |
| | | NC_005085 | NP_902262.1 |
| CV3489 | Chromobacterium violaceum ATCC 12472 | AE016922 | AAQ61150.1 |
| | | NC_005085 | NP_903159.1 |
| CV3323 (CbpD1) | Chromobacterium violaceum ATCC 12472 | AE016921 | AAQ60987.1 |
| | | NC_005085 | NP_902993.1 |
| EF0362 | Enterococcus faecalis V583 | AE016948 | AAO80225.1 |
| | | NC_004668 | NP_814154.1 |
| Sequence 4287 from U.S. Pat. No. | Enterococcus faecium | — | AAQ43729.1 |
| 6,583,275 | | | |
| FTL_1408 | Francisella tularensis subsp. holarctica LVS | AM233362 | CAJ79847.1 |
| FTT0816c | Francisella tularensis subsp. tularensis Schu 4 | AJ749949 | CAG45449.1 |
| HCH_00807 | Hahella chejuensis KCTC 2396 | CP000155 | ABC27701.1 |
| HCH_03973 | Hahella chejuensis KCTC 2396 | CP000155 | ABC30692.1 |
| Ip_1697 | Lactobacillus plantarum WCFS1 | AL935256 | CAD64126.1 |
| | | NC_004567 | NP_785278.1 |
| LSA1008 | Lactobacillus sakei subsp. sakei 23K | CR936503 | CAI55310.1 |
| YucG | Lactococcus lactis subsp. lactis IL1403 | AE006425 | AAK06049.1 |
| | | NC_002662 | NP_268108.1 |
| Ipp0257 | Legionella pneumophila Paris | CR628336 | CAH11404.1 |
| Lin2611 | Listeria innocua | AL596173 | CAC97838.1 |
| | | NC_003212 | NP_471941.1 |
| Lmo2467 | Listeria monocytogenes EGD-e | AL591983 | CAD00545.1 |
| | | NC_003210 | NP_465990.1 |
| LMOf2365_2440 | Listeria monocytogenes str. 4b F2365 | AE017330 | AAT05205.1 |
| OB0810 | Oceanobacillus iheyensis HTE831 | AP004595 | BAC12766.1 |
| | | NC_004193 | NP_691731.1 |
| PBPRB0312 | Photobacterium profundum SS9 | CR378676 | CAG22185.1 |
| plu2352 | Photorhabdus luminescens subsp. laumondii TTO1 | BX571866 | CAE14645.1 |
| | | NC_005126 | NP_929598.1 |
| Sequence 6555 from U.S. Pat. No. | Proteus mirabilis | — | AAR43285.1 |
| 6,605,709 | | | |
| chitin-binding protein ChiB | Pseudoalteromonas sp. S9 | AF007895 | AAC79666.1 |
| chitin-binding protein (CbpD; PA0852) | Pseudomonas aeruginosa PAO1 | AE004520 | AAG04241.1 |
| | | NC_002516 | NP_249543.1 |
| chitin-binding protein (CbpD) | Pseudomonas aeruginosa PAO25 | AF196565 | AAF12807.1 |
| PFL_2090 | Pseudomonas fluorescens Pf-5 | CP000076 | AAY91365.1 |
| Pfl_3569 | Pseudomonas fluorescens PfO-1 | CP000094 | ABA75307.1 |
| Psyr_2856 | Pseudomonas syringae pv. syringae B728a | CP000075 | AAY37892.1 |
| PSPTO2978 | Pseudomonas syringae pv. tomato str. DC3000 | AE016866 | AAO56470.1 |
| | | NC_004578 | NP_792775.1 |
| RF_0708 | Rickettsia fells URRWXCal2 | CP000053 | AAY61559.1 |
| chitin-binding protein (CbpA) | Saccharophagus degradans 2-40 | BK001045 | DAA01337.1 |
| ORF | Salinivibrio costicola 5SM-1 | AY207003 | AAP42509.1 |
| chitin-binding protein (Cbp21) | Serratia marcescens 2170 | AB015998 | BAA31569.1 |
| chitin-binding protein (Cbp21) | Serratia marcescens BJL200 | AY665558 | AAU88202.1 |
| ORF2 | Serratia marcescens KCTC2172 | L38484 | AAC37123.1 |
| SO1072 | Shewanella oneidensis MR-1 | AE015551 | AAN54144.1 |
| | | NC_004347 | NP_716699.1 |
| SG1515 (possible fragment) | Sodalis glossinidius str. ‘morsitans’ | AP008232 | BAE74790.1 |
| SAV6560 | Streptomyces avermitilis MA-4680 | AP005047 | BAC74271.1 |
| | | NC_003155 | NP_827736.1 |
| SAV2168 | Streptomyces avermitilis MA-4680 | AP005029 | BAC69879.1 |
| | | NC_003155 | NP_823344.1 |
| SAV5223 (Chb) | Streptomyces avermitilis MA-4680 | AP005042 | BAC72935.1 |
| | | NC_003155 | NP_826400.1 |
| SAV2254 (CelS2) | Streptomyces avermitilis MA-4680 | AP005030 | BAC69965.1 |
| | | NC_003155 | NP_823430.1 |
| SCO7225 or SC2H12.24 | Streptomyces coelicolor A3(2) | AL359215 | CAB94648.1 |
| | | NC_003888 | NP_631281.1 |
| SCO6345 or SC3A7.13 | Streptomyces coelicolor A3(2) | AL031155 | CAA20076.1 |
| | | NC_003888 | NP_630437.1 |
| SCO2833 (Chb) | Streptomyces coelicolor A3(2) | AL136058 | CAB65563.1 |
| | | NC_003888 | NP_627062.1 |
| SCO0643 or SCF91.03c | Streptomyces coelicolor A3(2) | AL132973 | CAB61160.1 |
| | | NC_003888 | NP_624952.1 |
| SCO0481 or SCF80.02 | Streptomyces coelicolor A3(2) | AB017013 | BAA75647.1 |
| | | AL121719 | CAB57190.1 |
| | | NC_003888 | NP_624799.1 |
| SCO1734 or SCI11.23 | Streptomyces coelicolor A3(2) | AL096849 | CAB50949.1 |
| | | NC_003888 | NP_626007.1 |
| CelS2 (SCO1188 or SCG11A.19) | Streptomyces coelicolor A3(2) | AL133210 | CAB61600.1 |
| | | NC_003888 | NP_625478.1 |
| chitin binding protein | Streptomyces griseus | AB023785 | BAA86267.1 |
| cellulose binding protein (ORF2) | Streptomyces halstedii | U51222 | AAC45430.1 |
| chitin-binding protein | Streptomyces olivaceoviridis ATCC 11238 | X78535 | CAA55284.1 |
| chitin binding protein (Chb2) | Streptomyces reticuli | Y14315 | CAA74695.1 |
| chitin-binding protein (Cbp1) | Streptomyces thermoviolaceus OPC-520 | AB110078 | BAD01591.1 |
| chitin-binding protein celS2 | Streptomyces viridosporus | AF126376 | AAD27623.1 |
| Tfu_1665 (E8) | Thermobifida fusca YX | CP000088 | AAZ55700.1 |
| Tfu_1268 (E7) | Thermobifida fusca YX | CP000088 | AAZ55306.1 |
| VCA0140 | Vibrio cholerae N16961 | AE004355 | AAF96053.1 |
| | | NC_002506 | NP_232540.1 |
| VCA0811 | Vibrio cholerae N16961 | AE004409 | AAF96709.1 |
| | | NC_002506 | NP_233197.1 |
| VFA0143 | Vibrio fischeri ES114 | CP000021 | AAW87213.1 |
| VFA0013 | Vibrio fischeri ES114 | CP000021 | AAW87083.1 |
| VPA0092 | Vibrio parahaemolyticus RIMD 2210633 | AP005084 | BAC61435.1 |
| | | NC_004605 | NP_799602.1 |
| VPA1598 | Vibrio parahaemolyticus RIMD 2210633 | AP005089 | BAC62941.1 |
| | | NC_004605 | NP_801108.1 |
| VV21258 | Vibrio vulnificus CMCP6 | AE016812 | AAO08152.1 |
| | | NC_004460 | NP_763162.1 |
| VV20044 | Vibrio vulnificus CMCP6 | AE016808 | AAO07021.1 |
| | | NC_004460 | NP_762031.1 |
| VVA0086 | Vibrio vulnificus YJ016 | AP005344 | BAC96112.1 |
| | | NC_005140 | NP_936142.1 |
| VVA0551 | Vibrio vulnificus YJ016 | AP005346 | BAC96577.1 |
| | | NC_005140 | NP_936607.1 |
| ChiY | Yersinia enterocolitica (type 0:8) WA-314 | AJ344214 | CAC83040.2 |
| YP0706 | Yersinia pestis biovar Medievalis str. 91001 | AE017129 | AAS60972.1 |
| | | NC_005810 | NP_992095.1 |
| YPO3227 | Yersinia pestis CO92 | AJ414156 | CAC92462.1 |
| | | NC_003143 | NP_406699.1 |
| Y0962 | Yersinia pestis KIM | AE013699 | AAM84543.1 |
| | | NC_004088 | NP_668292.1 |
| YPTB3366 | Yersinia pseudotuberculosis IP 32953 | BX936398 | CAH22604.1 |
| YPTB0899 | Yersinia pseudotuberculosis IP 32953 | BX936398 | CAH20139.1 |

EUKARYOTA

| | | | |
|---|---|---|---|
| ORF-26 | Agrotis segetum nucleopolyhedrovirus | DQ123841 | AAZ38192.1 |
| spheroidin-like protein (Gp 37) | Autographa californica nucleopolyhedrovirus | L22858 | AAA66694.1 |
| | | D00583 | BAA00461.1 |
| | | NC_001623 | NP_054094.1 |
| fusolin | Bombyx mor nuclear polyhedrosis virus | U55071 | AAB47606.1 |
| | | L33180 | AAC63737.1 |
| | | NC_001962 | NP_047468.1 |
| spheroidin | Choristoneura biennis entomopoxvirus | M34140 | AAA42887.1 |

VIRUSES

| | | | |
|---|---|---|---|
| ORF-26 | Agrotis segetum nucleopolyhedrovirus | DQ123841 | AAZ38192.1 |
| Spheroidin-like protein (Gp 37) | Autographa californica nucleopolyhedrovirus | D00583 | BAA00461.1 |
| | | L22858 | AAA66694.1 |
| | | NC_001623 | NP 054094.1 |
| Fusolin | Bombyx mori nuclear polyhedrosis virus | U55071 | AAB47606.1 |
| | | NC_001962 | NP_047468.1 |
| | | L33180 | AAC63737.1 |
| Spheroidin | Choristoneur biennis entomopoxvirus | M341140 | AAA42887.1 |
| ORF60 | Choristoneura fumiferana defective | AY327402 | AAQ91667.1 |
| | nucleopolyhedrovirus | | |
| | | NC_005137 | NP_932669.1 |
| spindle-like protein | Choristoneura fumiferana nuclear polyhedrosis | U26734 | AAC55636.1 |
| | virus | | |
| | | NC_004778 | NP_848371.1 |
| GP37 (ORF-67 GP37) | Chrysodeixis chalcites nucleopolyhedrovirus | AY864330 | AAY83998.1 |
| ORF57 | Epiphyas postvittana nucleopolyhedrovirus | AY043265 | AAK85621.1 |
| | | NC_003083 | NP_203226.1 |
| GP37 | Helicoverpa armigera single nucleocapsid | AF266696 | AAK57880.1 |
| | polyhedrovirus | | |
| | | AF303045 | AAK96305.1 |
| | | NC_003094 | NP_203613.1 |
| ORF59 | Helicoverpa zea nucleopolyhedrovirus | AF334030 | AAL56204.1 |
| | | NC_003349 | NP_542682.1 |
| gp37 | Heliocoverpa armigera nucleopolyhedrovirus G4 | AF271059 | AAG53801.1 |
| | | NC_002654 | NP_075127.1 |
| fusolin | Heliothis armigera entomopoxvirus | L08077 | AAA92858.1 |
| HynVgp086 (slp) | Hyphantria cunea nucleopolyhedrovirus | AP009046 | BAE72375.1 |
| Gp37 protein | Leucania separata nuclear polyhedrosis virus | AB009614 | BAA24259.1 |
| fusolin-like protein | Lymantria dispar nucleopolyhedrovirus | U38895 | AAB07702.1 |
| | | AF081810 | AA070254.1 |
| | | NC_001973 | NP_047705.1 |
| gp37 protein | Mamestra brassicae nucleopolyhedrovirus | AF108960 | AAD45231.1 |
| ORF 37 (Gp37) | Mamestra configurata nucleopolyhedrovirus A | U59461 | AAM09145.1 |
| | | AF539999 | AAQ11056.1 |
| Gp37 | Mamestra configurata nucleopolyhedrovirus B | AY126275 | AAM95019.1 |
| | | NC_004117 | NP_689207.1 |
| spheroidin-like protein (Gp 37) | Orgyia pseudotsugata nuclear polyhedrosis virus | U75930 | AAC59068.1 |
| | | D13306 | BAA02566.1 |
| | | NC_001875 | NP_046225.1 |
| enhancing factor | Pseudaletia separata entomopoxvirus | D50590 | BAA09138.1 |
| ORF25 | Spodoptera exigua nucleopolyhedrovirus | AF169823 | AAF33555.1 |
| | | NC_002169 | NP_037785.1 |
| gp37 (fragment) | Spodoptera frugiperda MNPV | AY250076 | AAP79107.1 |
| ubiquitin GP37 fusion protein | Spodoptera litura nucleopolyhedrovirus G2 | AF325155 | AAL01718.1 |
| | | NC_003102 | NP_258300.1 |
| gp37 | Trichoplusia ni single nucleopolyhedrovirus | DQ017380 | AAZ67435.1 |
| fusolin | unidentified entomopoxvirus | X77616 | CAA54706.1 |
| ORF107 | Xestia c-nigrum granulovirus | AF162221 | AAF05221.1 |
| | | NC_002331 | NP_059255.1 |

Other preferred bacterial AA10 proteins include:

| | | | |
|---|---|---|---|
| Cfla_0175 | Cellulomonas flavigena DSM 20109 | ADG73094.1 | D5UGB1 |
| Cfla_0172 | Cellulomonas flavigena DSM 20109 | ADG73091.1 | D5UGA8 |
| Cfla_0316 | Cellulomonas flavigena DSM 20109 | ADG73234.1 | D5UH31 |
| Cfla_0490 | Cellulomonas flavigena DSM 20109 | ADG73405.1 | D5UHY1 |
| CJA_2191 (Cbp33A) | Cellvibrio japonicus Ueda107 | ACE83992.1 | B3PJ79 |
| CJA_3139 (cbp33/10B) | Cellvibrio japonicus Ueda107 | ACE84760.1 | B3PDT6 |

The LPMO can thus be or correspond to or comprise a naturally occurring AA9, AA10, AA11 or AA13 family protein or a biologically active fragment thereof. (Examples of LPMOs that may be used include ScLPMO10C, ScLPMO10B, SmLPMO10A, PcLPMO9D (sequences provided below, SEQ ID NOs. 1-8) and TaGH61A (also known as TaLPMO9A, U.S. Pat. No. 7,534,594, incorporated herein by reference.) In the alternative the LPMO may be a non-native variant as disclosed hereinafter.

Thus in a preferred aspect the LPMO for use in the methods described herein is a polypeptide which comprises an amino acid sequence as set forth in any one of SEQ ID Nos. 2, 4, 6 or 8 (optionally with or without the leader peptide, where present) (or encoded by a sequence as set forth in any one of SEQ ID Nos. 1, 3, 5 or 7) or a sequence with at least 30, 40, 50, 60, 70, 80, 90, 95, 97, 98 or 99% sequence identity thereto or a biologically active fragment thereof comprising at least 100 amino acids (preferably at least 200 or 300 amino acids) of said sequence.

In connection with amino acid sequences, “sequence identity”, refers to sequences which have the stated value when assessed using e.g. using the SWISS-PROT protein sequence databank using FASTA pep-cmp with a variable pamfactor and gap creation penalty set at 12.0 and gap extension penalty set at 4.0 and a window of 2 amino acids). Sequence identity at a particular residue is intended to include identical residues which have simply been derivatized. Sequence identity assessments are made with reference to the full length sequence of the recited sequence used for comparison.

Fragments as described herein are preferably at least 200, 300 or 400 amino acids in length and preferably comprise simple, short deletions from the N of C terminal e.g. a C-terminal deletion of 1, 2, 3, 4 or 5 amino acids.

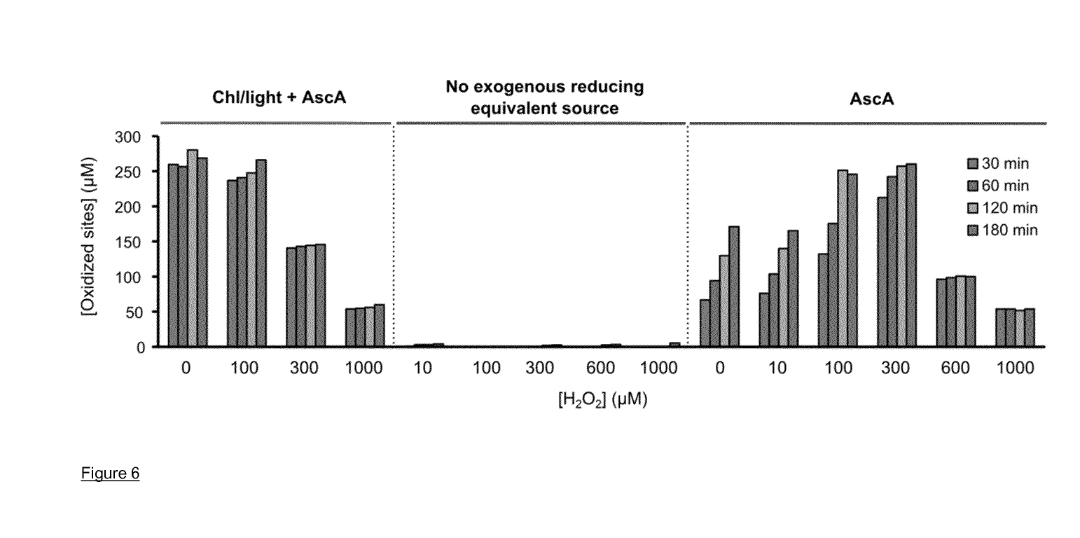

All such variants or fragments must retain the functional property of the protein from which they are derived such that they are “biologically active”. Thus they must retain LPMO activity, e.g. under the conditions described in the Examples (e.g. catalyze oxidative degradation of the polysaccharide substrate and exhibit enhanced activity when used in the presence of a reducing agent and hydrogen peroxide when compared to performing the method without the reducing agent and hydrogen peroxide, see e.g. FIGS. 6, 9 and 11). Some loss of activity is contemplated, e.g. the biologically active fragment or variant may have at least 50, 60, 70, 80, 90 or 95% of the LPMO activity of the native full length sequence wherein said activity may be assessed in terms of the extent or level of degradation achieved over a set time period, e.g. as assessed by the production of reaction products such as oxidized products or oligo and/or di-saccharides.

Variants include or comprise naturally occurring variants of the LPMOs described above such as comparable proteins or homologues found in other species or more particularly variants found within other microorganisms, which have the functional properties of the enzymes as described above.

Variants of the naturally occurring LPMOs as defined herein can also be generated synthetically e.g. by using standard molecular biology techniques that are known in the art, for example standard mutagenesis techniques such as site directed or random mutagenesis. Such variants further include or comprise proteins having at least 70, 80, 85, 90, 91, 92, 93, 94, 95, 96, 97, 98 or 99% sequence identity with a naturally occurring LPMO at the amino acid level.

When variants are generated, it should be noted that appropriate residues to modify depend on the properties that are being sought in such a variant. In the case that a variant having the same LPMO activity as the native parent molecule is being sought, the residues are in general those residues that are not involved in the catalytic reaction or interaction of the enzyme with the polysaccharide substrate (the Examples identify residues of importance to catalytic activity). However, those residues may be targeted, in the alternative, to develop variants with improved reactivity. This could be achieved by standard protein engineering techniques or by techniques based on random mutagenesis followed by screening, all techniques that are well known in the art. Attempts to improve the function of LPMOs may include improving the binding and catalytic ability of the enzyme, e.g. to act on other substrates, e.g. carbohydrate containing copolymers, e.g. protein-carbohydrate co-polymers. In light of the findings provided in the Examples, LPMO properties may be improved by mutations in or near the catalytic center of the LPMO that would improve the oxidative stability of the LPMO, preferably, the ability to withstand damage caused by H₂O₂-derived reactive oxygen species, such as a hydroxyl radical, generated by the LPMO itself.

A person skilled in the art will recognize the potential of using the native proteins' framework to create variants that are optimised for other insoluble polymeric polysaccharide substrates (e.g. other forms of chitin or cellulose), or insoluble carbohydrate-containing co-polymers.

Preferred “variants” include those in which instead of the naturally occurring amino acid the amino acid which appears in the sequence is a structural, e.g. non-native analogue thereof. Amino acids used in the sequences may also be derivatized or modified, e.g. labelled, glycosylated or methylated, providing the function of the LPMO is not significantly adversely affected.

Further preferred variants are those in which relative to the above described native amino acid sequences, the amino acid sequence has been modified by single or multiple amino acid (e.g. at 1 to 10, e.g. 1 to 5, preferably 1 or 2 residues) substitution, addition and/or deletion or chemical modification, including deglycosylation or glycosylation, but which nonetheless retain functional activity, insofar as they bind to the polysaccharide substrate and enhance its degradation, particularly when used in conjunction with one or more hydrolytic enzymes.

Within the meaning of “addition” variants are included amino and/or carboxyl terminal fusion proteins or polypeptides, comprising an additional protein or polypeptide or other molecule fused to the enzyme sequence. C-terminal fusions are preferred. It must of course be ensured that any such fusion to the enzyme does not adversely affect the functional properties required for use in the methods of the invention as set out herein.

“Substitution” variants preferably involve the replacement of one or more amino acids with the same number of amino acids and making conservative substitutions. Such functionally-equivalent variants mentioned above include in particular naturally occurring biological variations (e.g. found in other microbial species) and derivatives prepared using known techniques. In particular functionally equivalent variants of the LPMOs described herein extend to enzymes which are functional in (or present in), or derived from different genera or species than the specific molecules mentioned herein.

Variants such as those described above can be generated in any appropriate manner using techniques which are known and described in the art, for example using standard recombinant DNA technology.

As referred to herein a “reducing agent” is an element or compound in a redox (reduction-oxidation) reaction that reduces another species and in so doing becomes oxidized and is therefore the electron donor in the redox reaction. The reducing agent is also referred to herein as a reductant and is a molecule which delivers reducing equivalents. In one embodiment the reducing agent is non-enzymatic. In this particular invention, the species to be reduced is the copper in the catalytic domain of LPMO which is reduced from Cu(II) to Cu(I). The reducing agent functions as an electron donor in the enzymatic process. Preferably said reducing agent is ascorbic acid. Other reducing agents may be molecules such as enzymes or other chemical compounds. Thus, alternative reducing agents include reduced glutathione, Fe(II)SO₄, LiAlH₄, NaBH₄, lignin or a fragment thereof, a cellobiose dehydrogenase, a phenol, a glucose-methanol-choline oxidoreductase, superoxide, organic acids (such as succinic acid, gallic acid, coumaric acid, humic acid and ferulic acid) and reducing sugars (such as glucose, glucosamine and N-acetylglucosamine.) Other reducing agents that may be used include catechin and dithiothreitol. It will be appreciated that that any chemical compound or protein capable of reducing Cu(II) could be considered for use in the present invention and would be used as a reducing agent.

More than one of such agents may be used in line with methods of the invention and may be selected according to the substrate and conditions used (e.g. pH and temperature). It will be appreciated that the efficacy and stability of reducing agents varies between these agents and depends on pH. Thus the pH and reducing agent should be optimized for the LPMO to be used. It will also be appreciated that the amount of reducing agent in a reaction needs to be adapted to the amount of LPMO in that reaction.

As discussed hereinafter, reducing agents are preferably added or present to a final concentration range of 0.001 to 10 mM. It has been found, as described in the Examples, that the reducing agents may be used at catalytic rather than stoichiometric amounts as they are involved in priming the LPMO for further activity. Thus, in a preferred aspect, the method as described herein results in the release of oxidized products, and the concentration of the reducing agent is lower, preferably at least ten-fold lower than the concentration that would be necessary to achieve equivalent yields of oxidized products in reactions run under identical conditions but without hydrogen peroxide. Preferably the reducing agent is at a concentration of less than 200 μM, for example less than 100 μM, especially preferably between 10 and 100 μM. As discussed hereinafter, the reducing agent may be provided in another component used in the reaction, e.g. may be present in sufficient quantities in the biomass.

As referred to herein an “oxidized product” is the product of LPMO acting on a polysaccharide substrate to yield either (1) one normal non-reducing end and an oxidized “acidic” end (i.e. oxidized at C1) that, in the case of chitin is a 2-(Acetylamino)-2-deoxy-D-gluconic acid and in the case of cellulose is a D-gluconic acid (aldonic acid), or (2) one normal reducing end and an oxidized non-reducing end, which in the case of cellulose and chitin would be a 4-keto sugar, or, since some LPMOs have a mixed activity producing both types of oxidized products, (3) a mixture of all the aforementioned products. The amount of oxidized products which is present may be determined for example as described in the examples (using e.g. MALDI-TOF MS, for qualitative assessment, or HPAEC-PAD or HILIC-UV for quantitative assessment).

As noted above sub-stoichiometric levels of the reducing agent may be used. To quantify this, first the reaction may be run according to the method as claimed with reducing agent (amount x) and hydrogen peroxide. The reaction may then be re-run without the hydrogen peroxide and the reducing agent increased until equivalent yields to the first reaction are achieved (reducing agent amount y). Reactions may then be conducted, according to the invention, with reducing agent in an amount less than y/10 (which will include x), i.e. ten-fold lower that the amount that would be required if the same reaction was run without hydrogen peroxide.

As referred to herein a “means which generates hydrogen peroxide” is a collection of one or more molecules or components which together allow the generation of hydrogen peroxide under suitable conditions. A “part” of the means refers to one or more of these molecules or components. Thus, for example, said means may be an enzyme together with the one or more components required for its activity. Such enzymes include cellobiose dehydrogenase and certain single domain flavoenzymes. Substrates, co-factors, co-substrates and any other components required for activity for those enzymes comprise the means which generates hydrogen peroxide. (A co-factor or co-substrate refers to molecules which interact with the enzyme to enhance its catalytic function and which may be altered by the interaction with enzyme.) Any one or more of these various components and/or the enzyme may be supplied to the reaction, e.g. by addition to the reaction, or may be present in the enzyme preparation or in other material used in the reaction, e.g. in the biomass.

Means which generate hydrogen peroxide also encompasses chemical means. Preferably such a means comprises more than one molecule or component which allows a chemical reaction that produces hydrogen peroxide to be carried out. Such means may include, for example, superoxide, which is spontaneously converted to hydrogen peroxide. In this case the means may also include molecules, components or means which generate superoxide or assist in its conversion to hydrogen peroxide, e.g. photochemical, chemical or enzymatic means to generate superoxide or convert it to hydrogen peroxide. Chemical methods of generating superoxide include the use of KO₂. Enzymatic means of generating superoxide include the use of xanthine oxidase. Chemical methods of converting superoxide to H₂O₂ include the use of Mn(II)SO₄ in combination with phosphate or carbonate ions, or reductants. Enzymatic means of generating H₂O₂ include the use of superoxide dismutase to accelerate conversion of superoxide to hydrogen peroxide. Other methods of generating hydrogen peroxide may also be used including the use of electrochemistry (e.g. use of glassy-carbon electrodes on which a compound such as quinone is grafted), photocatalysis (using a photocatalyst such a titanium dioxide, TiO₂) or metal complexes (e.g. palladium complexes).

Another example of a means which generates hydrogen peroxide is a photoreactive compound which together with light allows its generation. As referred to herein a “photoreactive compound” is a compound that is activated by light to an extent that depends on reaction parameters such as the intensity and the wavelength of the light, the pH and the temperature. An example of this system is the Chl/light, Chl/light-AscA systems, e.g. as used in the Examples provided herein in which the LPMO is exposed to visible light in the presence of chlorophyllin (Chi). Light-exposed chlorophyllin produces superoxide which in turn can lead to production of hydrogen peroxide as discussed above. Preferably, but not essentially this is performed in the presence of a reducing agent such as ascorbic acid (AscA). Preferably in such methods, ascorbic acid is used at a concentration of less than 2 mM, preferably less than 1 mM, e.g. from 0.01 to 0.2 mM. It will be appreciated that selection of appropriate ranges for the various reactants takes into account the concentrations of the other reactants. For example, higher levels of reductants may be necessary if higher levels of substrate (polysaccharide) are employed, see hereinafter.

In such methods visible light is generally used, but light of lower or higher wavelengths is also contemplated. The reaction mixture may be irradiated continuously, or periodically (e.g. after monitoring) during all or part of the reaction. If used periodically the reaction may be irradiated for 30 seconds to 30 minutes at a time e.g. for 1-30 or 2-30 minutes at a time. The light intensity may be from 0.02-100 W·cm⁻². Both the light duration and intensity affect the production of H₂O₂ and thus may be selected and modified according to the reaction conditions.

In accordance with the invention the amount of hydrogen peroxide present during the degradation reaction is maintained in a concentration range at which the hydrogen peroxide acts as a co-substrate for said LPMO and said LPMO is inactivated (i) by no more than 20% during a) the reaction time required to achieve 40% (maximal) conversion of the polysaccharide or b) 4 hours of reaction time, (ii) by no more than 50% during a) the reaction time required to achieve 70% (maximal) conversion of the polysaccharide or b) 12 hours of reaction time or (iii) by no more than 20% when said LPMO is contacted with said concentration of hydrogen peroxide in the presence of said polysaccharide and reducing agent for 20 minutes.

As noted above it has surprisingly been found that hydrogen peroxide both activates LPMO at low concentrations, but at higher concentrations inactivates LPMO. Thus, the level of hydrogen peroxide in the reaction needs to be maintained at a concentration level that maximizes the activatory effects, but minimizes the inhibitory effects. This may be controlled in a number of ways as discussed hereinafter.

As referred to herein, “acting as a co-substrate” refers to the hydrogen peroxide being present in sufficient amounts that it positively influences the generation of oxidized products (i.e. increases the reaction rate of the LPMO) at that concentration.

At higher concentrations of hydrogen peroxide inactivation of the LPMO occurs. To maximize the reaction rate this must be avoided. Some inactivation of the LPMO may be tolerated, but must preferably not exceed 20% (or 50%) inactivation during (i) the reaction time required to achieve 40% (or 70%) (maximal) conversion of the polysaccharide or (ii) 4 (or 12) hours of reaction time, or alternatively expressed must not exceed 20% inactivation when the LPMO is contacted with the concentration of hydrogen peroxide in question in the presence of said polysaccharide and reducing agent (as used in the method) for 20 minutes. In the above definition the time over which the inactivation is assessed, in one alternative, is determined by % conversion that is achieved during that time. The 40 or 70% conversion referred to herein refers to the % of the maximal conversion that could be achieved using the enzymes of the reaction (as described hereinbefore) if no inactivation or inhibition occurred. Alternatively the time over which inactivation is to be assessed is denoted in hours over which the reaction is conducted.

One or more of the alternatives defining inactivation may be satisfied. In relation to the first option (wherein inactivation does not exceed 20% during the time required to achieve 40% conversion or 4 hours of reaction time), preferably the inactivation is less than 10%, e.g. from 5-10%. Preferably the time over which this is measured is the reaction time required to achieve at least 40%, e.g. at least 50, 60 or 70% (maximal) conversion or at least 4 hours of reaction time, e.g. at least 8, 12, 16, 24, 36, 48 or 60 hours. In relation to the second option (wherein inactivation does not exceed 50% during the time required to achieve 70% conversion or 12 hours of reaction time), preferably the inactivation is less than 40%, e.g. from 5-30%. Preferably the time over which this is measured is the reaction time required to achieve at least 70%, e.g. at least 80 or 90% (maximal) conversion or at least 12 hours of reaction time, e.g. at least 16, 24, 36, 48 or 60 hours. In relation to the third option (wherein inactivation does not exceed 20% during a 20 minute test reaction), preferably the inactivation is less than 10%, e.g. from 5-10%. Thus, any reactions in which the LPMO is inactivated by more than 50%, e.g. by 60, 70, 80, 90 or 100% fall outside the scope of the invention.

In some instances, instead of using maximal conversion values, particularly if hydrolytic enzymes are also used, the conversion figures above may alternatively be absolute conversion values. In that case, if LPMO is used alone, the same level of inactivation applies, but the absolute conversion achieved (i.e. total number of glycosidic bonds cleaved in the substrate) may be considered 10% in which case the inactivation is assessed over the time required to achieve 4 or 7% absolute conversion. If LPMO is used together with hydrolytic enzymes absolute conversion may be from 50-100% in which case inactivation is assessed over the time required to achieve 25-50% absolute conversion. In a further alternative, instead of maximal conversion or absolute conversion values, the reaction time required to achieve 40 or 70% of maximum (achievable) yield (e.g. oxidized products) may be used to assess inactivation (no more than 20 or 50%, respectively). The same preferred % values apply to these figures as apply to the related figures indicated above.

As noted above in a preferred aspect LPMO is used together with hydrolytic enzymes. In this scenario, in a preferred aspect, the invention provides a method of enzymatically degrading a polysaccharide comprising contacting said polysaccharide with one or more lytic polysaccharide monooxygenase (LPMO), additionally comprising contacting said polysaccharide (or the degradation product thereof) with one or more hydrolytic enzymes, wherein said enzymatic degradation is carried out in a reaction in the presence of:

a) at least one reducing agent; and

b) hydrogen peroxide or a means which generates hydrogen peroxide, wherein the amount of hydrogen peroxide present during the degradation reaction is maintained in a concentration range at which the hydrogen peroxide acts as a co-substrate for said LPMO and said LPMO is inactivated by

(i) no more than 20% during a) the reaction time required to achieve 40% absolute conversion of the polysaccharide or b) 4 hours of reaction time,

(ii) no more than 50% during a) the reaction time required to achieve 70% absolute conversion of the polysaccharide or b) 12 hours of reaction time, or

(iii) no more than 20% when said LPMO is contacted with said concentration of hydrogen peroxide in the presence of said polysaccharide and reducing agent for 20 minutes. In this case, maximal or absolute (as indicated) conversion rates may be used. The 40% absolute conversion indicates that the reaction time is defined by the time it takes for 40% of the total 100% of the polysaccharide's glycosidic bonds to be cleaved.

The percent of inactivation of the enzyme may be assessed in a number of ways. As discussed above activity of the LPMO (or enzyme mixtures) may be assessed in terms of the extent or level of degradation achieved over a set time period, e.g. as assessed by the production of reaction products such as oxidized products or oligo and/or di-saccharides (i.e. examination of product yield). Preferably the amount of oxidized products produced are assessed as a measure of LPMO activity.

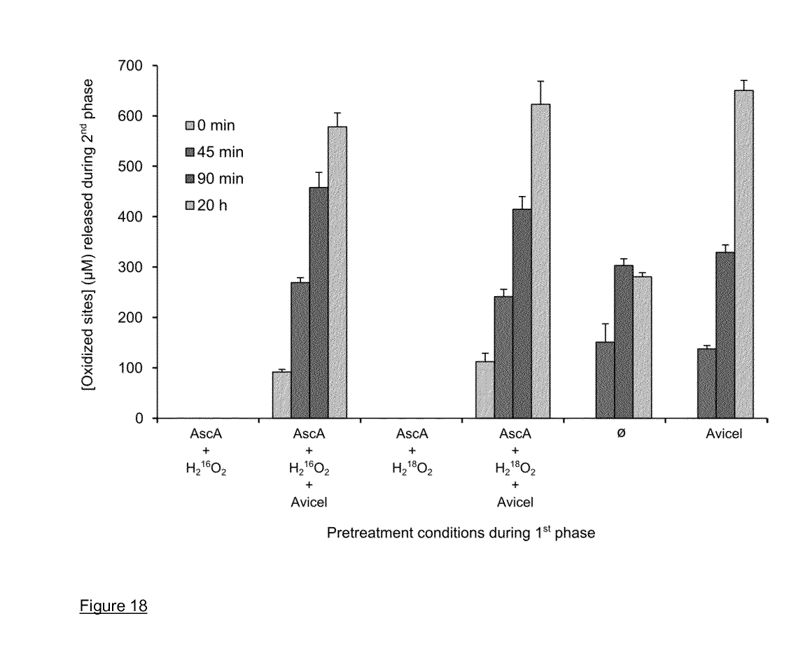

Alternatively the number of glycosidic bonds cleaved may be assessed (i.e. examination of conversion of the starting material). Inactivation to the extent of 20% is equivalent to 80% remaining activity, i.e. a comparison of the efficacy of the enzyme at the start and the end of the assessment period indicates that the enzyme produces products or converts the starting material 20% less efficiently at the end of the assessment period. For example the degree of inactivation may be assessed as in the examples, in which a sample of the reaction may be taken and tested for LPMO activity at the start, during and at the end of the reaction or reaction times indicated above by assessing the enzyme kinetics of the LPMO (see e.g. FIG. 18) and comparing the activity at the different time points.

As discussed hereinbefore, alternative methods of the invention do not define the extent of inactivation of LPMO. For example, reference is made to the LPMO not being substantially inactivated. This is intended to mean that the LPMO is able to act catalytically and generate oxidized products even if without optimal performance. Another alternative is to maintain a steady amount of hydrogen peroxide, by keeping it within a narrow range, i.e. does not vary by more than 5%. Reference is also made to controlling the LPMO activity by adjusting the concentration of the different reactants. This is intended to mean that the concentration is adjusted such that LPMO is subject to appropriate levels of hydrogen peroxide to optimize activity as described herein. Adjustment may be made by additions or removals as described herein. Optimized production of oxidation products refers to the best possible rate of production of those products under the reaction conditions used.

In the reaction mix various components affect the amount of hydrogen peroxide that may be tolerated. As described in the examples, the level of the reductant affects the levels of hydrogen peroxide which are present. The presence of substrate (e.g. biomass) is protective to the LPMO. The LPMO itself may produce hydrogen peroxide. Considering the sequence variation in natural LPMOs, natural LPMOs may vary both in terms to the rate of hydrogen peroxide production and their sensitivity to inactivation by certain hydrogen peroxide concentration. Various components present in the reaction mix may increase or decrease the level of hydrogen peroxide that is present. Thus, in any particular system, the specific level of hydrogen peroxide that is ideal will vary depending on the nature and amounts of all the various components and molecules which are present. Thus, for each system to be used, the level of hydrogen peroxide which should be used should be assessed based on its ability to act as a co-substrate and its inactivation effect on LPMO during the course of the reaction. This ensures that the ratio between the different components is maintained at appropriate levels. As the amounts of the various components or molecules will likely change during the course of the reaction (e.g. be generated or used up) ideally the level of hydrogen peroxide is monitored during the reaction to ensure that for the particular components or molecules that are being used it remains within the desired concentration range.

It will be understood that the concentrations of various components discussed above and used in the examples should be adapted to the substrate concentration, sometimes referred to as Dry Matter concentration in industrial bioprocessing. In the examples, generally the DM concentration used is 1%, whereas in industrial processes, the DM concentration typically would be 15-30% or from 5 to 15%, e.g. around 10%. When such high DM concentrations are used, also higher concentrations of the other reactants, the LPMO, the other enzymes, the reductant and hydrogen peroxide will be needed (e.g. 5-10, e.g. 10-fold more).

As discussed above, there are different ways of maintaining the hydrogen peroxide within the desired concentration range to maximize its catalytic activity and minimize its negative effects on LPMO. Thus, for example, one may change the concentration of one or more of said (i) polysaccharide, (ii) one or more LPMO, (iii) at least one reducing agent, and (iv) hydrogen peroxide or means which generates hydrogen peroxide, during said reaction. As discussed herein the ratio between the different components is important in view of their interplay during the reaction.

Whilst the appropriate concentration of hydrogen peroxide to be used should be assessed based on its influence on the LPMO, in one embodiment the concentration range for hydrogen peroxide in the reaction is 2 to 200 μM, for example 1 to 100 μM. In some embodiment the hydrogen peroxide in the reaction after administration may be as low as 1 μM or lower, and thus in another option the hydrogen peroxide in the reaction is from 0.01 to 10 μM, e.g. from 0.1 to 1 μM. Thus the hydrogen peroxide in the reaction may be in the range of 0.01 to 200 μM. Preferably the concentration of hydrogen peroxide does not vary by more than said 5, 10, 20 or 30% during the course of the reaction.

Conveniently, the concentration of hydrogen peroxide in the reaction mix may be maintained by supply to the reaction at an average rate of 0.2 to 500 μM hydrogen peroxide per minute, preferably 0.5 to 20 μM hydrogen peroxide per minute. It will be appreciated that the amount to be used will depend on the levels of the other reactants. For example when high substrate concentrations or dry matter amounts are used, enzyme concentrations, including the LPMO concentration, would normally be higher and the hydrogen peroxide should be supplied at a higher rate. For example, when the substrate's dry matter concentration is high, e.g. above 10%, higher levels of hydrogen peroxide may be used, e.g. from 0.5 to 50 or 150 μM per minute. This supply includes provision at various intervals (which may be regular or irregular, e.g. in response to monitoring) or continuously. The result of irregular provision is that the concentration of hydrogen peroxide will vary during the reaction, but this is acceptable providing it is maintained within the defined concentration range which ensures that the desired ratio between the different components is maintained. The hydrogen peroxide may be supplied by directly providing hydrogen peroxide or by making changes that will affect the level of hydrogen peroxide in the reaction.

Due to the interplay between the various components of the reaction the hydrogen peroxide may be maintained in the desired range in a number of different ways. Conveniently the concentration range may be maintained by

(i) addition and/or removal of said hydrogen peroxide or said means which generates hydrogen peroxide, or a part thereof;

(ii) addition and/or removal of a means to remove hydrogen peroxide;

(iii) addition and/or removal of said one or more LPMO;

(iv) addition and/or removal of said at least one reducing agent; and/or

(v) addition and/or removal of said polysaccharide, during the degradation reaction.

As referred to herein “addition” refers to active administration of the entity of interest to the reaction or by causing its generation within the reaction. In the former case any convenient means may be used, e.g. manual addition or addition with a pump (including automated methods reliant on detection of hydrogen peroxide levels with a probe). In the latter case, for example, a reducing agent may be generated by initiating a reaction which results in its generation. Addition also encompasses activating a relevant pathway that results in production of a desired molecule, e.g. activating an enzyme that produces a product of interest. “Removal” encompasses both physical removal of the entity of interest as well as its inactivation, e.g. inactivation of any enzyme, whether reversibly or irreversibly.

Hydrogen peroxide may be added directly to the reaction as a liquid in the described concentration, e.g. as described above.

By way of example, the means which generates hydrogen peroxide may be an enzyme and one or more components required for the activity of said enzyme. In this case the concentration range is maintained by addition or removal of the enzyme or one or more components required for its activity. Preferably the one or more components are selected from a co-factor or substrate for said enzyme. Thus one may remove a means for generating hydrogen peroxide by inactivating a relevant enzyme or removing required co-factors or substrates.

In another alternative the means which generates hydrogen peroxide may be a photoreactive compound and light and in that case the concentration range may be maintained by

(i) addition and/or removal of the photoreactive compound;

(ii) altering the duration and/or intensity and/or wavelength of light which irradiates the photoreactive compound;

and/or

(iii) addition and/or removal of the at least one reducing agent.

To alter the ratios between the different components, the addition or removal of a means to remove hydrogen peroxide is also contemplated. Such a means may be chemical or involve an enzyme, for example a peroxidase, peroxyredoxin, peroxygenase or catalase which converts hydrogen peroxide into a different molecule hence effectively removing it from the system.

The concentration of LPMO and/or reducing agent may alternatively or additionally be changed during the degradation reaction by the addition or removal of said LPMO and/or reducing agent and/or a component which affects the concentration of said LPMO and/or reducing agent. As noted above, the polysaccharide may be added or removed during the reaction. As disclosed herein, the ratio between the LPMO and the amount of substrate, i.e. the amount of LPMO binding sites on the substrate, affects LPMO stability, especially in reactions that also contain hydrogen peroxide.

The above described steps of addition and removal allow for fine control of the ratios of the different components in the reaction mix and hence optimize the effect of H₂O₂ on the LPMO. This can be achieved by varying the levels of just one component in the reaction mix during the reaction or by altering the levels of more than one component. For example, the substrate and LPMO may be added in batches while controlling the other components or the substrate and LPMO may be added in bulk and the other components varied during the reaction.

Thus, the additions and/or removals are performed one or more times during the reaction, preferably two or more times during the reaction, for example 3, 4, 5 or more times (e.g. 10, 30 or 50 or more times). The additions or removals may be at regular or irregular intervals. Preferably, the one or more of the additions or removals may be performed continuously.

Conveniently the level of hydrogen peroxide is monitored one or more times during the reaction e.g. as described in the Examples. (Various hydrogen peroxide probes are also known in the art (e.g. Dulcotest® sensors from ProMinent or probes from AMT Analysenmesstecknik GmbH). Commercial kits for detecting hydrogen peroxide may also be used (e.g. Amplex Red®).) For example the level may be monitored regularly or irregularly throughout the reaction, e.g. 2 or more, e.g. 3, 5, 10 or more times. Conveniently, monitoring may be conducted continuously.

Whilst the hydrogen peroxide may be monitored, it is also appropriate to consider the state or concentration of one or more components of the reaction as an indicator of the level of hydrogen peroxide. For example one may monitor the activity of the LPMO. If LPMO is failing to produce oxidized products (e.g. over a certain time period) this is evidence that inactivation has occurred or that hydrogen peroxide or reductant levels are depleted. To address this, further LPMO, hydrogen peroxide and/or reductant may be added. Similarly the levels of reductants may be monitored during the reaction.

As discussed herein and as illustrated in the Examples, LPMOs do not use molecular oxygen as a substrate and thus the reaction may be conducted under anaerobic conditions. Thus, in a preferred aspect, the concentration of dissolved molecular oxygen is reduced relative to the concentration of dissolved molecular oxygen present under aerobic conditions. Aerobic conditions refer to conducting the reaction with free access to air or other oxygen-containing gas. The concentration of dissolved molecular oxygen may be reduced by adopting partial or fully anaerobic conditions. Mechanisms for reducing molecular oxygen from reactions are well known, e.g. reaction systems (including the airspace and reaction mixes) may be flushed with gases that do not contain oxygen (e.g. N₂) for several hours (e.g. from 4 to 24 hours), optionally under vacuum. In a preferred feature, the methods described herein may be conducted under anaerobic conditions.

Other enzymes are known which have active sites which are structurally related to the active sites of LPMOs and which have previously been thought to use O₂ as their co-substrate and to require two electrons to complete a catalytic cycle. In particular, the copper-binding site, so-called histidine-brace (Quinlan et al., 2011, 2011, Proc. Natl. Acad. Sci. USA., 108, 15079-15084), is conserved in methane mono-oxygenase (MMO), catalyzing the conversion of methane to methanol. Other mechanistically-related enzymes are dopamine β-mono-oxygenase (DβM), peptidyl-glycine α-hydroxylating mono-oxygenases (PHM) or tyramine β-mono-oxygenase (TβM), which all catalyze C—H bond hydroxylation of their respective substrates (neurotransmitters or hormones, medically-relevant) and are described as requiring O₂ and 2 electrons. Those proteins contain non-coupled binuclear copper centers, where a single coordinated copper is thought to activate O₂. In light of their relationship to LPMO it is expected that the true co-substrate for these enzymes is H₂O₂. In a further aspect therefore the present invention provides a method of enhancing the activity of one of the above described enzymes by contacting the enzyme with hydrogen peroxide or a means which generates hydrogen peroxide and a reducing agent wherein the amount of hydrogen peroxide present during the reaction is maintained in a concentration range at which the hydrogen peroxide acts as co-substrate but does not inactivate the enzyme by more than 20 or 40% during the reaction.

The following description sets out conditions that can be used for performance of the method of the invention, but it should be noted that any appropriate conditions can be used.

Prior to contacting the polysaccharide-containing material with the LPMO, the polysaccharide-containing material may be pre-treated.

The polysaccharide-containing material may be pre-treated, e.g. to disrupt plant cell wall components, using conventional methods known in the art. Prior to pre-treatment, where appropriate, the polysaccharide-containing material may be subjected to pre-soaking, wetting, or conditioning using methods known in the art. Physical pre-treatment techniques include, for example, various types of milling, irradiation, steaming/steam explosion and hydrothermolysis; chemical pre-treatment techniques can include dilute acid, alkaline (e.g. lime pre-treatment), organic solvent (such as organosolv pre-treatments), ammonia treatments (e.g. ammonia percolation (APR) and ammonia fibre/freeze explosion (AFEX)), sulfur dioxide, carbon dioxide, wet oxidation and pH-controlled hydrothermolysis; and biological pre-treatment techniques can involve applying lignin-solubilizing microorganisms (see, for example, Hsu, 1996, Pre-treatment of biomass, in “Handbook on Bioethanol: Production and Utilization”, Wyman, ed., Taylor & Francis, Washington, D.C., 179-212; Ghosh & Singh, 1993, Adv. Appl. Microbiol., 39, 295-333; McMillan, 1994, Pretreating lignocellulosic biomass: a review, in “Enzymatic Conversion of Biomass for Fuels Production”, Himmel et al. eds., ACS Symposium Series 566, American Chemical Society, Washington, D.C., Chapter 15; Gong et al., 1999, Advances in Biochemical Engineering/Biotechnology, Scheper, ed., Springer-Verlag Berlin Heidelberg, Germany, 65, 207-241; Olsson & Hahn-Hagerdal, 1996, Enz. Microb. Tech., 18, 312-331; and Vallander & Eriksson, 1990, Adv. Biochem. Eng./Biotechnol., 42, 63-95). Additional pre-treatments include ultrasound, electroporation, microwave, supercritical CO₂, supercritical H₂O and ammonia percolation.

Pre-treated Corn Stover is a cellulose-containing material derived from corn stover, e.g. by treatment with heat and dilute acid.

Following optional pre-treatment, the polysaccharide-containing material (the substrate) may be exposed to the LPMO in vitro in any appropriate vessel, e.g. by mixing together the substrate (polysaccharide) and the enzyme in an appropriate medium (e.g. a solution, such as an aqueous solution) or by applying the enzyme to the substrate (e.g. by applying the enzyme in a solution to a substrate).

In a preferred embodiment the LPMO is present in a buffer such as a phosphate buffer, e.g. a sodium phosphate buffer, or Tris buffer. Conveniently the pH may be controlled by a pH-stat. Suitable concentration ranges for such a buffer are 1-100 mM. The LPMO may be provided as a purified preparation (as described hereinafter) or may be present in a composition, wherein it may be a major component, preferably comprising at least 20, 30, 40, 50, 60 or 70% w/w dry weight in the composition, or it may be a minor component (e.g. in a mixture with one or more hydrolytic enzymes), preferably comprising at least 1, 2, 5 or 10%, e.g. 1-5%, w/w dry weight in the composition.

The LPMO can be present in the solution at any suitable concentration, such as a concentration of 0.001-1.0 mg/ml, e.g. 0.01-0.1 mg/ml or 0.05-0.5 mg/ml.

In a preferred aspect the one or more LPMO is present in the reaction in the amount of 0.005 to 2 g per kg of polysaccharide, preferably from 0.01 to 1 g per kg of polysaccharide.

The polysaccharide substrate is present in the reaction mix at any suitable concentration which will depend to some extent on the purity of the polysaccharide in the material containing it. Conveniently, however, the polysaccharide itself is present at a concentration of from 5 to 250 mg/ml, preferably 10 to 200 mg/ml, or more preferably 25 to 250 mg/ml, especially preferably at least 25 mg/ml. Preferably the polysaccharide is present in the material containing the polysaccharide to a level of >40%, e.g. >50, 60, 70, 80 or 90%, w/w dry weight in the material.

Preferably the polysaccharide substrate is exposed to the one or more enzymes used in the reaction, e.g. by incubation together, for a period of 2, 4, 6, 12 or 24 hours or more, such as 4-24 or 6-24 hours, e.g. 36 or 48 hours or more, or 3, 4, 5, 6, 7, 8, 9, 10, 11, 12, 13, 14 days or more. In a preferred aspect the incubation is 6-24 hours. This incubation is in general carried out at or about 50° C., although appropriate temperatures for optimizing the enhancement of polysaccharide degradation can readily be determined by the skilled person in the art. For example, the temperature can be in the range of 20-65° C., e.g. 30-60° C., preferably 40-55° C.

It will be appreciated that the necessary incubation times, pH, temperature, substrate and enzyme concentrations are not independent of each other. Thus, a large range of conditions can be envisaged, which can easily be evaluated. The LPMOs serve to enhance degradation by the hydrolytic enzymes and thus may allow the use of lower concentrations of the latter or shorter reaction times.

Preferably a pH in the range of 4 to 10 is used. Preferably the pH is in the range of 4.5 to 8.5 or 5-8. The preferred pH is about pH 6 to 8, e.g. at pH 7.0. In the alternative, particularly when enzyme mixtures are used in the methods described herein, the pH range is from 4 to 6, e.g. from 4.5 to 5.5, e.g. preferably pH 5.0 is used.

The reducing agent may be added to the reaction mix or may be present in the reaction mix by virtue of one of the components present in or generated in the reaction mix. For example, the substrate which is used, which may be a biomass, may contain sufficient reducing agent for the reaction. As discussed herein the method of the invention allows the use of low levels of reducing agent and thus small amounts provided in other materials used or generated in the reaction may be sufficient. When the reducing agent is added to the reaction mix it is preferably added for the duration of the degradation reaction, though it may be added after that reaction has commenced and may be present only while the LPMO is present or active. Reducing agents are preferably added or present to a final concentration range of 0.001 to 10 mM, preferably 0.01 to 2 mM, especially preferably 0.01-1 mM. Preferably, the reducing agent is at a concentration of less than 200 μM, preferably less than 100 μM, especially preferably between 10 and 100 μM.

As noted above, reducing agents may be present in the polysaccharide substrate, e.g. lignin present in a lignocellulosic biomass, but preferably said reducing agents are added to the reaction mix.

LPMOs need copper ions and under some conditions it may be necessary to add small amounts of copper(II) salts, preferably Cu(II)SO₄, to make sure that there is sufficient copper for the LPMOs. The molar amount or concentration of the copper(II) salt will be equal to or lower than the amount or concentration of the LPMO.

Preferably the incubation is carried out with agitation, particularly when a cellulose-containing material is used.

In a preferred aspect, the LPMO is used at a concentration of 0.01 to 0.5 mg/ml and the polysaccharide substrate at 25 to 250 mg/ml (when calculated according to the target substrate content and not taking into account the additional material that may be present with the substrate) and the reaction is conducted at pH 6-8 for 6 to 24 hours at 40 to 55° C.

In methods in which the degradation is carried out with the LPMO only, the result of said reaction is incomplete degradation (depolymerization) of the polysaccharide to yield largely insoluble long oligosaccharides and minor fractions of soluble oligosaccharides, perhaps including very minor fractions of disaccharides. Preferably said degradation is enhanced further or completed by the use of appropriate additional degradative glycoside hydrolases.

Thus in a further preferred aspect the present invention provides a method of enzymatically degrading a polysaccharide comprising

a) contacting said polysaccharide with one or more LPMOs, wherein said degradation or hydrolysis is carried out in the presence of at least one reducing agent and hydrogen peroxide or a means which generates hydrogen peroxide as defined hereinbefore, and

b) contacting said polysaccharide (or the degradation product thereof) with one or more hydrolytic enzymes, preferably a cellulose hydrolase or chitin hydrolase.

Clearly in performing the method the LPMO and the hydrolytic enzyme must be selected in accordance with the polysaccharide substrate, e.g. AA9 and a cellulose hydrolase for cellulose and chitin-active AA10 and a chitin hydrolase for chitin (though cross-reaction between different substrates does occur).

It will be obvious to the expert in the field that polysaccharides such as chitin and, especially, cellulose may occur in complex co-polymeric matrices including for example hemicelluloses in the case of plant cell wall material. Since cellulose and hemicelluloses interact strongly, it is possible that loosening of the cellulose structure by an LPMO may make not only the cellulose but also the hemicellulose more accessible for attack by appropriate saccharolytic enzymes, and vice versa. Thus, cellulose-active LPMOs may also be used concomitantly with e.g. hemicellulases or other enzymes targeting the non-chitin and non-cellulose polymers in complex chitin- or cellulose- containing co-polymeric materials, in order to increase the hydrolytic efficiency of these enzymes. Likewise, hemicellulose-active LPMOs may be combined with cellulases.

As referred to herein a “hydrolytic enzyme” is an enzyme which is capable of cleaving glycosidic bonds between saccharide monomers or dimers in a polysaccharide, using a standard hydrolytic mechanism as employed by most enzymes classified in the glycoside hydrolase (GH) families in the CAZy database. These enzymes include cellulose hydrolases, chitin hydrolases, ß-glucosidases, hemicellulases and amylases.

As referred to herein a “cellulose hydrolase” is an enzyme which hydrolyses cellulose or intermediate breakdown products. Preferably the hydrolase is a cellulase. Cellulases are classified as glycosyl hydrolases (GH) in families based on their degree of identity and fall within various GH families, including families 1, 3, 5-9, 12, 44, 45, 48 and 74. Based on mechanism they can be grouped into exo-1,4-ß-D-glucanases or cellobiohydrolases (CBHs, EC 3.2.1.91), endo-1,4-ß-D-glucanases (EGs, EC 3.2.1.4) and ß-glucosidases (RGs, EC 3.2.1.21). EGs cleave glycosidic bonds within cellulose microfibrils, acting preferentially at amorphous cellulose regions. EGs fragment cellulose chains to generate reactive ends for CBHs, which act “processively” to degrade cellulose, including crystalline cellulose, from either the reducing (CBH1) or non-reducing (CBHII) ends, to generate mainly cellobiose. Cellobiose is a water-soluble beta-1, 4-linked dimer of glucose. Beta-glucosidases hydrolyze cellobiose to glucose.

The ability of cellulose hydrolases to hydrolyse cellulose may be assessed by using methods known in the art, including methods in which non-modified cellulose is used as substrate. Activity is then measured by measuring released products, using either HPLC-based methods or methods that determine the number of newly formed reducing ends (e.g. Zhang et al, 2009, Methods Mol. Biol., 2009, 581, p 213-31; Zhang et al., 2006, Biotechnol. Adv., 24(5), p 452-81). In the alternative, the efficacy of the cellulose hydrolase may be assessed by using an appropriate substrate and determining whether the viscosity of the incubation mixture decreases during the reaction. The resulting reduction in viscosity may be determined by a vibration viscosimeter (e.g. MIVI 3000 from Sofraser, France). Determination of cellulase activity, measured in terms of Cellulase Viscosity Unit (CEVU), quantifies the amount of catalytic activity present in a sample by measuring the ability of the sample to reduce the viscosity of a solution of the substrate.

Cellulases may be obtained from commercial sources, i.e. companies such as Novozymes, DuPont and DSM. One example of such a cellulase cocktail is Cellic® CTec2 as used in the Examples. Alternatively cellulases may be produced using standard recombinant techniques for protein expression. The scientific literature contains numerous examples of the cloning, overexpression, purification and subsequent application of all types of cellulases.

Cellulase mixtures may be used, e.g. a cellulase mixture which comprises at least one endoglucanase, a cellobiohydrolase moving towards the reducing end, a cellobiohydrolase moving towards the non-reducing end, and a beta-glucosidase. More preferably, more complex mixtures are used, in particular mixtures containing several endoglucanases with different substrate specificities (e.g. acting at different faces of the cellulose crystals). Appropriate cellulases may be readily identified taking into account the substrate to be degraded.

As referred to herein a “chitin hydrolase” is an enzyme which hydrolyses chitin or intermediate breakdown products. Preferably said chitin hydrolase is a chitinase, chitobiase, chitosanase or lysozyme. The degradation may be complete or partial. For example, the activity of some chitin hydrolases, e.g. chitinases on chitin substrates is not strong enough to result in complete degradation of the substrate. This is particularly the case for chitinases such as ChiG from Streptomyces coelicolor that do not have their own CBM, or chitinases such as ChiB from S. marcescens. In this case, the use of a LPMO enzyme that acts on chitin in accordance with the present invention can result in enhanced chitin degradation and preferentially result in complete degradation that was not previously possible. Other chitinases, such as ChiC from S. marcescens, are capable of completely degrading chitin, but the speed of this process increases upon addition of an LPMO such as CBP21.

Chitinase enzymes are found in plants, microorganisms and animals. Chitinases have been cloned from various species of microorganisms and have been categorised into two distinct families, designated family GH18 and family GH19 of the glycoside hydrolases, based on sequence similarities (Henrissat and Bairoch, 1993, Biochem, J. 293:781-788). Chitobiases occur in family GH2O. Chitosanases are found in several families, including families GH46 and GH75. These enzymes are referred to collectively herein as chitin hydrolases.

There are several ways to measure chitinase activity that are well known in the field, including methods in which non-modified chitin is used as substrate. Activity on non-modified chitin is measured by measuring released products, using either HPLC-based methods or methods that determine the number of newly formed reducing ends.

Chitinases may be obtained from commercial sources, i.e. companies such as Sigma. Alternatively chitinases may be produced using standard recombinant techniques for protein expression. The scientific literature contains numerous examples of the cloning, overexpression, purification and subsequent application of all types of chitinases (e.g. Horn et al., 2006, FEBS J., 273(3), p 491-503 and references therein).

Other suitable hydrolytic enzymes for hydrolysing additional non-cellulose (or non-chitin) polysaccharides include hemicellulases such as xylanases, arabinofurosidases, feruloyl esterases, glucuronidases and mannanases.

In addition, other enzymes which aid degradation or hydrolysis of the substrate polysaccharide may be used, including enzymes acting on non-polysaccharide biomass components such as lignin, for example, enzymes selected from, peroxidases, laccases or esterases, may also be used.

ß-glucosidases may be used to remove soluble short oligosaccharides (particularly disaccharides) which may inhibit glycoside hydrolases and to provide monomers which are desirable for downstream processing (see hereinbelow). By way of example, for cellulose a ß-glucosidase(s) may be used and for chitin a ß-N-acetylglucosaminidase(s) (also known as chitobiase) may be used.

Thus, a further aspect of the invention provides a method as defined herein additionally comprising contacting said polysaccharide (or the degradation product thereof) with one or more hydrolytic enzymes, preferably a cellulose hydrolase or chitin hydrolase, and optionally contacting said polysaccharide (or the degradation product thereof) with one or more enzymes selected from ß-glucosidases, hemicellulases, amylases, peroxidases, laccases or esterases. In one embodiment the enzymes are contacted with said polysaccharide simultaneously with said LPMO, but alternative administration protocols are contemplated as described hereinafter.

Whilst the use of native hydrolytic and other enzymes described herein is preferred, variants defined in accordance with the properties described hereinbefore for the LPMO's variants may also be used.

Preferably, when said polysaccharide is cellulose, said hydrolytic enzyme is an endo-1,4-ß-D-glucanase optionally used in combination with other 1,4-ß-D-glucanases such as cellobiohydrolases and/or a ß-glucosidases.

Thus, the enzymes to be used in methods of the invention may be selected based on the polysaccharide substrate to be hydrolysed.

For example preferred combinations for chitin hydrolysis are AA10 or AA11 family proteins (e.g. SmLPMO10A (also known as CBP21)) as the LPMO (or variants or fragments thereof) with one or more chitinase, e.g. ChiA, ChiB, ChiC and ChiG.

When the substrate is cellulose, the LPMO is preferably an AA9 family protein (as described herein), though in view of their ability to act on cellulose, AA10 family proteins may also be used. Appropriate hydrolytic enzymes may be selected from known enzymes, e.g. cellulases as described hereinbefore.

In a preferred aspect two or more LPMOs are employed in the methods of the invention, e.g. 2, 3 or 4 LPMOs. In view of their preferred substrate specificities, enhanced degradative effects may be expected when used together (Forsberg et al., 2014, Proc. Natl. Acad. Sci. USA, 111(23), 8446-8451. Thus, for example, one may use two or more AA10 family proteins and/or two or more AA9 family proteins (as described herein).

Appropriate enzymes for use in accordance with the invention can be determined by use of screening techniques to assess in vitro hydrolysis, e.g. as described in the Examples.

To identify LPMOs which may be used in combination, the enzymes may be assessed to determine whether their activity will achieve enhanced effects on the substrate. For convenience, various forms of chitin (e.g. alpha chitin or beta-chitin) or cellulose (e.g. various types of cellulose fibers, cellulose pulps, filter paper, microcrystalline cellulose, Avicel, Carboxymethylcellulose) may be used for easy experimentation. Industrially relevant biomasses such as sulfite-pulped Norway spruce or steam exploded birch may also be used. See WO2012/019151 (which is incorporated herein by reference) for more detailed descriptions of how to select LPMOs and preferred LPMOs (in which CBM33 and GH61 family proteins correspond to AA10 and AA9 family proteins, respectively) for use alone or in combination. Other issues that should be taken into account include the LPMO's sensitivity to oxidative inactivation. The Examples identify histidines which are vulnerable to auto-oxidation. For example, many fungal LPMOs are known to carry a methylation of their N-terminal histidine which is likely to provide protection against oxidative inactivation. Other features of the catalytic center, such as the nature of the amino acids near the copper-binding site could also affect the sensitivity to hydrogen peroxide. Furthermore, one should take into account the affinity of the LPMO for H₂O₂ and its substrate in solution and the ability of the LPMO to generate H₂O₂ in solution. As will be appreciated, many of these processes and interactions will also depend on commonly varied process parameters such as pH, temperature, water content, or ionic strength. The processes and interactions will also depend on other factors such as the dry matter content in the reaction and the cellulose concentration in the reaction mixture.

In the methods described above using both an LPMO and one or more additional enzymes, preferably hydrolytic enzymes, the step with the LPMO is carried out under conditions which allow the enzyme to interact or bind to the polysaccharide as described hereinbefore. The same conditions and considerations are applied to the additional step using additional enzymes, which step may be carried out simultaneously or subsequent to the first step. In total the incubation may be conducted for 3, 4, 5, 6, 7, 8, 9, 10, 11, 12, 13, 14 days or more, but is typically performed for preferably about 8 to about 96 hours, more preferably about 8 to about 72 hours and most preferably about 8 to about 48 hours or 4 to 24 hours.

Preferably aqueous solutions of the enzymes are used and preferably the enzymatic treatment is carried out in a suitable aqueous environment under conditions that can be readily determined by one skilled in the art.

Each enzyme used in the methods may be provided as a purified preparation (as described hereinafter) or may be present in a composition, (e.g. including the other enzymes for use in the methods) preferably at least 0.5, 1, 2, 5 or 10%, preferably 1-5% w/w dry weight in the composition.

For the methods described hereinbefore the enzymatic treatment can be carried out as a fed batch or continuous process where the polysaccharide-containing material (substrate), which may be pre-treated, is fed gradually to, for example, an enzyme containing solution.

The depolymerization (saccharification) is generally performed in stirred-tank reactors or fermentors under controlled pH, temperature and mixing conditions as discussed hereinbefore. Suitable process time, temperature and pH conditions can readily be determined by one skilled in the art and are discussed hereinbefore and can depend on the substrate and enzymes used and their concentrations and the concentration of reductant and hydrogen peroxide and whether the substrate has been pretreated and whether a fermenting organism is included, see hereinbelow.

The dry solids content is in the range of preferably about 5 to about 40 wt %, more preferably about 10 to about 30 wt % and most preferably about 15 to about 30 wt %.

Each enzyme used in the reaction can be present in the solution at any suitable concentration, such as a concentration of 0.01-5.0 mg/ml, e.g. 0.1-2.0 mg/ml. Alternatively expressed, the enzymes may be used at a concentration of 0.1-20 mg enzyme/g of polysaccharide substrate, e.g. 1-10 mg/g substrate. A typical total enzyme concentration for LPMOs and all other enzymes combined would be in the range of 0.5-15 mg/g polysaccharide substrate. Suitable concentrations can be determined depending on the substrate and the material containing the substrate and the conditions of the reaction, e.g. temperature, pH and duration.

The steps in which the LPMO and the additional enzyme(s) are contacted with the polysaccharide substrate may be performed separately or together or a combination thereof, e.g. the LPMO enzyme may be added and after an initial incubation period the additional (e.g. hydrolytic) enzyme(s) may be added. (If more than one additional enzyme is used, they may be added separately or sequentially.) One or more additions of the additional enzyme(s) may be made. In the alternative, the LPMO may be removed (e.g. physically or effectively, such as by inactivation) before any additional enzyme is added. Any steps in which the LPMO is not present (e.g. a step in which only a cellulase is used) need not be conducted in the presence of a reducing agent or hydrogen peroxide.

Other enzymes may also be added in addition to or as an alternative to the chitin or cellulose hydrolytic enzymes discussed above, depending on the nature of the substrate that is to be degraded. For example, if the polysaccharide to be degraded is a copolymer which contains protein, proteases may also be added. Suitable examples include Alcalase, Neutrase, Papain and other broad-specificity proteolytic enzymes. In each experimental set-up the suitability of proteases will need to be checked, especially if other enzymes (e.g. chitinases or cellulases), which may be destroyed by some of the available proteases, are present simultaneously.

The LPMOs and other, e.g. hydrolytic, enzymes for use in the methods of the invention may be isolated, extracted or purified from various different sources or synthesised by various different means. As mentioned above the enzymes may be provided in purified preparations or in the presence of other components.

Chemical syntheses may be performed by methods well known in the art involving, in the case of peptides, cyclic sets of reactions of selection deprotection of the functional groups of a terminal amino acid and coupling of selectively protected amino acid residues, followed finally by complete deprotection of all functional groups. Synthesis may be performed in solution or on a solid support using suitable solid phases known in the art, such as the well known Merrifield solid phase synthesis procedure.

In one embodiment the enzymes for use in the invention are substantially purified, e.g. more than 70%, especially preferably more than 90% pure (as assessed for example, in the case of peptides or proteins, by an appropriate technique such as peptide mapping, sequencing or chromatography or gel electrophoresis).

Purification may be performed for example by chromatography (e.g. HPLC, size-exclusion, ion-exchange, affinity, hydrophobic interaction, reverse-phase) or capillary electrophoresis. Notwithstanding the before, use of less pure preparations of LPMOs and other enzymes may also be used to carry out the reactions described.

Recombinant expression of proteins is also well known in the art and an appropriate nucleic acid sequence can be used to express the enzymes used herein for subsequent expression and optional purification using techniques that are well known in the art. For example, an appropriate nucleic acid sequence can be operably linked to a promoter for expression of the enzyme to be used in bacterial cells, e.g. E coli which may then be isolated or if the enzyme is secreted, the culture medium or the host expressing the enzyme may be used as the source of the enzyme.

The methods described above have applications in a number of different fields in which depolymerisation of polysaccharides forms one of the method steps or in which the products of that hydrolysis are useful.

Thus in a further aspect the present invention provides a method of producing soluble saccharides, wherein said method comprises degrading a polysaccharide by a method as described hereinbefore, wherein said degradation releases said soluble saccharides.

The result of complete hydrolysis is soluble sugars. Usually, a mixture of monomeric sugars and higher order oligosaccharides (e.g. disaccharides) are generated. As discussed above, preferably ß-glucosidases are used to produce monomeric sugars and thus their use in methods of the invention is preferred. The partially or completed degraded polysaccharide-containing material is preferably recovered for further processing, e.g. fermentation. Soluble products of degradation of the polysaccharide-containing material can be separated from the insoluble material using technology well known in the art such as centrifugation, filtration and gravity settling.

Preferably said soluble saccharides are isolated or recovered after said degradation or hydrolysis process. Preferably the soluble saccharides which are isolated or recovered are chitobiose and/or N-acetylglucosamine (from chitin) or cellobiose and/or glucose (from cellulose) and/or oligosaccharides thereof.

N-acetylglucosamine and oligosaccharides of N-acetylglucosamine have a number of commercial uses including use as a food supplement. Chitin fragments have found utility in various applications including use as immune stimulants (Aam et al., 2010, Marine Drugs, 8(5), 1482-517).

The soluble saccharides resulting from hydrolysis of cellulose have various applications, particularly for use as a source of energy in fermentation reactions.

Preferably the saccharide mixture released after hydrolysis containing monomeric sugars is fermented to generate an organic substance such as an alcohol, e.g. ethanol.

Thus the present invention further provides a method of producing an organic substance, preferably an alcohol, comprising the steps of:

i) degrading a polysaccharide by a method as described hereinbefore to produce a solution comprising soluble saccharides;

ii) fermenting said soluble saccharides, preferably with one or more fermenting microorganisms, to produce said organic substance as the fermentation product; and optionally

iii) recovering said organic substance.

Optionally, said soluble saccharides produced in step (i) may be isolated or purified from said solution.

The organic substance thus produced forms a further aspect of the invention.

As referred to herein “soluble saccharides” include monosaccharides, disaccharides and oligosaccharides which are water soluble, preferably mono- and/or disaccharides. Preferably said soluble saccharides are fermentable, e.g. glucose, xylose, xylulose, arabinose, maltose, mannose, galactose and/or soluble oligosaccharides.

“Fermentation” refers to any fermentation process or any process comprising a fermentation step.

The above method may additionally comprise the use of one or more additional enzymes such as esterases (e.g. lipases, phospholipases and/or cutinases), proteases, laccases and peroxidases.

The steps of hydrolysis (saccharification) and fermentation may be performed separately and/or simultaneously and include, but are not limited to, separate hydrolysis and fermentation (SHF), simultaneous saccharification and fermentation (SSF), simultaneous saccharification and cofermentation (SSCF), hybrid hydrolysis and fermentation (HHF), separate hydrolysis and co-fermentation (SHCF), hybrid hydrolysis and cofermentation (HHCF) and direct microbial conversion (DMC). Conveniently, any method known in the art comprising pre-treatment, enzymatic hydrolysis (saccharification), fermentation, or a combination thereof, can be used in the practicing of the above methods.

Conveniently, a conventional apparatus can include a fed-batch stirred reactor, a batch stirred reactor, a continuous flow stirred reactor with ultrafiltration and/or a continuous plug-flow column reactor (de Castilhos Corazza et al, 2003, Acta Scientiarum. Technology, 25, 33-38; Gusakov & Sinitsyn, 1985, Enz. Microb. Technol., 7, 346-352), an attrition reactor (Ryu & Lee, 1983, Biotechnol. Bioeng., 25, 53-65), or a reactor with intensive stirring induced by an electromagnetic field (Gusakov et al., 1996, Appl. Biochem. Biotechnol. 56, 141-153). Additional reactor types include, for example, fluidized bed, upflow blanket, immobilized and extruder type reactors for hydrolysis and/or fermentation.

Pre-treatments that may be used were discussed hereinbefore and apply to all methods of the invention. The polysaccharide-containing material can be pre-treated before hydrolysis and/or fermentation. Pre-treatment is preferably performed prior to the hydrolysis step. Alternatively, the pretreatment can be carried out simultaneously with hydrolysis, such as simultaneously with treatment of the polysaccharide-containing material with the enzymes used in the methods (i.e. LPMO and other enzymes, including hydrolytic enzymes) to release fermentable sugars, such as glucose and/or cellobiose. In most cases the pre-treatment step itself results in some conversion of biomass to fermentable sugars (even in the absence of enzymes).

The fermentable sugars obtained by the method of the invention can be fermented by one or more fermenting microorganisms capable of fermenting the sugars directly or indirectly into a desired fermentation product.

The fermentation conditions depend on the desired fermentation product and fermenting organism and can easily be determined by one skilled in the art.

In the fermentation step, sugars, released from the substrate are fermented to a product, e.g. ethanol, by a fermenting organism, such as yeast. The polysaccharide substrate to be used in the method may be selected based on the desired fermentation product.

The “fermenting microorganism” refers to any microorganism, including bacterial and fungal organisms, suitable for use in the fermentation process to produce a fermentation product. The fermenting organism can be a C6 sugar fermenting organism a C5 sugar fermenting organisms, an organism that can ferment both sugar types, or a combination of these organisms. Both C6 and C5 fermenting organisms are well known in the art. Suitable fermenting microorganisms are able to ferment, i.e., convert, sugars, such as glucose, xylose, xylulose, arabinose, maltose, mannose, galactose, or oligosaccharides, directly or indirectly into the desired fermentation product.

Examples of bacterial and fungal fermenting organisms producing ethanol are described by Lin et al., 2006, Appl. Microbiol. Biotechnol., 69, 627-642.

Examples of fermenting microorganisms that can ferment C6 sugars include bacterial and fungal organisms, such as yeast. Preferred yeast includes strains of Saccharomyces spp., preferably Saccharomyces cerevisiae.

Examples of fermenting organisms that can ferment C5 sugars include bacterial and fungal organisms, such as yeast. Preferred C5 fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis or Candida utilis.

Other fermenting organisms include strains of Zymomonas, such as Zymomonas mobilis; Hansenula, such as Hansenula anomala; Klyveromyces, such as K. fragilis; Schizosaccharomyces, such as S. pombe; and E. coli, especially E. coli strains that have been genetically modified to improve the yield of ethanol.

In a preferred aspect, the yeast is a Saccharomyces spp. In a more preferred aspect, the yeast is Saccharomyces cerevisiae, Saccharomyces distaticus, Saccharomyces uvarum. In another preferred aspect, the yeast is a Kluyveromyces, e.g. Kluyveromyces marxianus or Kluyveromyces fragilis.

Other yeast that may be used include Clavispora, e.g. Clavispora lusitaniae or Clavispora opuntiae; Pachysolen, e.g. Pachysolen tannophilus; and Bretannomyces, e.g. Bretannomyces clausenii.

Bacteria that can efficiently ferment hexose and pentose to ethanol include, for example, Zymomonas, such as Zymomonas mobilis and Clostridium, such as Clostridium thermocellum.

Commercially available yeast suitable for ethanol production include, e.g. ETHANOL RED™ yeast (available from Fermentis/Lesaffre, USA), FALI™ (available from Fleischmann's Yeast, USA), SUPERSTART™ and THERMOSACC™ fresh yeast (available from Ethanol Technology, WI, USA), BIOFERM™ AFT and XR (available from NABC—North American Bioproducts Corporation, GA, USA), GERT STRAND™ (available from Gert Strand AB, Sweden) and FERMIOL™ (available from DSM Specialties).

The fermenting microorganism(s) is typically added to the degraded polysaccharide-material and the fermentation is performed for about 8 to about 96 hours, such as about 24 to about 60 hours. The temperature is typically between about 26° C. to about 60° C., in particular about 32° C. to 50° C. and at about pH 3 to about pH 8, such as around pH 4-5, 6, or 7. The above conditions will of course depend on various factors including the fermenting microorganism that is used.

The fermenting microorganism(s) is preferably applied in amounts of approximately 10⁵ to 10¹², preferably from approximately 10⁷ to 10¹⁰, especially approximately 2×10⁸ viable cell count per ml of fermentation broth.

Various fermentation products may be produced. In one embodiment the fermenting organism may be the product itself, e.g. certain yeast cells may be used in animal or fish feed. Alternatively the product is produced during fermentation. Where appropriate, the fermenting microorganism may be tailored to produce fermentation products, such as speciality or platform chemicals (which may be used for a broad range of technologies).

For ethanol production, following the fermentation the fermented slurry is distilled to extract the ethanol. The ethanol obtained according to the methods of the invention can be used as, e.g. fuel ethanol, drinking ethanol, i.e., potable neutral spirits, or industrial ethanol.

A fermentation stimulator can be used in combination with any of the enzymatic processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin and Vitamins A, B, C, D and E.

The organic substance which is the fermentation product can be any substance derived from the fermentation. The fermentation product can be, without limitation, an alcohol (e.g. arabinitol, butanol, ethanol, glycerol, methanol, 1,3-propanediol, sorbitol or xylitol); an organic acid (e.g. acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid or xylonic acid); a ketone (e.g. acetone); an aldehyde (e.g. formaldehyde); an amino acid (e.g. aspartic acid, glutamic acid, glycine, lysine, serine or threonine); or a gas (e.g. methane, hydrogen (H₂), carbon dioxide (CO₂) or carbon monoxide (CO)). The fermentation product can also be protein.

In a preferred aspect, the fermentation product is an alcohol. It will be understood that the term “alcohol” encompasses a substance that contains one or more hydroxyl moieties. Preferably the alcohol is arabinitol, butanol, ethanol, glycerol, methanol, 1,3-propanediol, sorbitol or xylitol. Ethanol is the preferred product.

The fermentation product(s) may be recovered from the fermentation medium using any method known in the art including, but not limited to, chromatography (e.g. ion exchange, affinity, hydrophobic, chromatofocusing and size exclusion), electrophoretic procedures (e.g. preparative isoelectric focusing), differential solubility (e.g. ammonium sulfate precipitation), distillation or extraction. For example, ethanol is separated from the fermented cellulose-containing material and purified by conventional methods of distillation. Ethanol with a purity of up to about 96 vol. % can be obtained.

SmLPMO10A
DNA sequence -
>gi|52854326|gb|AY665558.1| Serratia marcescens
strain BJL200 chitin-binding protein precursor,
gene, complete cds
SEQ ID NO: 1
ATGAACAAAACTTCCCGTACCCTGCTCTCTCTGGGCCTGCTGAGCGCGGC
CATGTTCGGCGTTTCGCAACAGGCGAATGCCCACGGTTATGTCGAATCGC
CGGCCAGCCGCGCCTATCAGTGCAAACTGCAGCTCAACACGCAGTGCGGC
AGCGTGCAGTACGAACCGCAGAGCGTCGAGGGCCTGAAAGGCTTCCCGCA
GGCCGGCCCGGCTGACGGCCATATCGCCAGCGCCGACAAGTCCACCTTCT
TCGAACTGGATCAGCAAACGCCGACGCGCTGGAACAAGCTCAACCTGAAA
ACCGGTCCGAACTCCTTTACCTGGAAGCTGACCGCGCGTCACAGCACCAC
CAGCTGGCGCTATTTCATCACCAAGCCGAACTGGGACGCTTCGCAGCCGC
TGACCCGCGCTTCCTTTGACCTGACGCCGTTCTGCCAGTTCAACGACGGC
GGCGCCATCCCTGCCGCACAGGTCACCCACCAGTGCAACATACCGGCAGA
TCGCAGCGGTTCGCACGTGATCCTTGCCGTGTGGGACATAGCCGACACCG
CTAACGCCTTCTATCAGGCGATCGACGTCAACCTGAGCAAATAA
Amino acid sequence (underlined, the signal
peptide not present in mature protein) -
>tr|O8300|O83009_SERMA CBP21 OS = Serratia
marcescens GN = cbp PE = 1 SV = 1
SEQ ID NO: 2
MNKTSRTLLSLGLLSAAMFGVSQQANAHGYVESPASRAYQCKLQLNTQCG
SVQYEPQSVEGLKGFPQAGPADGHIASADKSTFFELDQQTPTRWNKLNLK
TGPNSFTWKLTARHSTTSWRYFITKPNWDASQPLTRASFDLTPFCQFNDG
GAIPAAQVTHQCNIPADRSGSHVILAVWDIADTANAFYQAIDVNLSK
ScLPMO10C
DNA sequence -
>gi|24413740:58454-59548 Streptomyces
coelicolor A3(2) complete genome;
segment 5/29
SEQ ID NO: 3
ATGGTTCGACGCACCAGACTCCTCACCCTCGCGGCGGTACTGGCCACCCT
GCTCGGCTCGCTCGGCGTGACCCTTCTGCTCGGGCAGGGGCGGGCCGAGG
CGCACGGCGTGGCGATGATGCCCGGCTCCCGCACCTACCTGTGCCAGCTG
GACGCCAAGACCGGCACCGGCGCCCTCGACCCGACGAACCCCGCCTGCCA
GGCCGCCCTCGACCAGAGCGGGGCGACGGCCCTGTACAACTGGTTCGCCG
TGCTCGACTCCAACGCGGGGGGCCGCGGCGCCGGTTACGTGCCGGACGGC
ACCCTGTGCAGCGCCGGCGACCGTTCCCCGTACGACTTCTCCGCCTACAA
CGCCGCCCGCTCGGACTGGCCCCGCACGCACCTGACGTCGGGTGCGACGA
TCCCGGTGGAATACAGCAACTGGGCGGCCCACCCCGGGGACTTCCGGGTG
TACCTGACCAAGCCGGGCTGGTCGCCCACGTCCGAGCTGGGCTGGGACGA
CCTGGAGCTGATCCAGACGGTGACCAACCCGCCCCAGCAGGGCTCGCCGG
GCACCGACGGGGGCCACTACTACTGGGACCTCGCGCTGCCCTCGGGCCGC
TCGGGCGACGCGTTGATCTTCATGCAGTGGGTGCGTTCGGACAGCCAGGA
GAACTTCTTCTCCTGCTCGGACGTCGTCTTCGACGGCGGCAACGGAGAGG
TCACCGGCATCCGCGGTTCCGGGAGCACCCCGGACCCGGACCCGACACCG
ACCCCGACGGACCCGACCACCCCGCCCACGCACACCGGCTCCTGCATGGC
CGTGTACTCGGTGGAGAACTCCTGGAGCGGCGGCTTCCAGGGGTCGGTCG
AGGTGATGAACCACGGCACCGAGCCGCTGAACGGCTGGGCCGTGCAGTGG
CAGCCGGGCGGCGGGACCACGCTCGGCGGGGTGTGGAACGGTTCGCTGAC
CAGCGGCTCCGACGGTACGGTCACGGTCCGCAACGTGGACCACAACCGCG
TCGTACCACCGGACGGGAGCGTGACCTTCGGCTTCACCGCCACTTCGACG
GGCAATGACTTCCCGGTCGACTCGATCGGCTGCGTGGCACCCTGA
Amino acid sequence (underlined, the signal
peptide not present in mature protein) -
>tr|Q9RJY2|Q9RJY2_STRCO Putative secreted
cellulose binding protein OS = Streptomyces
coelicolor (strain ATCC BAA-471/A3(2)/M145)
GN = SCO1188 PE = 1 SV = 1
SEQ ID NO: 4
MVRRTRLLTLAAVLATLLGSLGVTLLLGQGRAEAHGVAMMPGSRTYLCQL
DAKTGTGALDPTNPACQAALDQSGATALYNWFAVLDSNAGGRGAGYVPDG
TLCSAGDRSPYDFSAYNAARSDWPRTHLTSGATIPVEYSNWAAHPGDFRV
YLTKPGWSPTSELGWDDLELIQTVTNPPQQGSPGTDGGHYYWDLALPSGR
SGDALIFMQWVRSDSQENFFSCSDVVFDGGNGEVTGIRGSGSTPDPDPTP
TPTDPTTPPTHTGSCMAVYSVENSWSGGFQGSVEVMNHGTEPLNGWAVQW
QPGGGTTLGGVWNGSLTSGSDGTVTVRNVDHNRVVPPDGSVTFGFTATST
GNDFPVDSIGCVAP
ScLPMO10B
DNA sequence -
>gi|24413728:94459-95145 Streptomyces
coelicolor A3(2) complete genome;
segment 3/29
SEQ ID NO: 5
TCAGCCGAAGTCGACGTCGCTGCACAGGAAGTAGGTCTGGTCCATGTGCG
AGGCCTGCCAGATCGTGTAGACGACGTGCCGGCCGGTCAGTCCCGAGGTA
CTCACCGGGATCTCGTAGTTCTGGCTGGGCCCGTAGCTGCCCGTGCGCGC
GACCTGCTGGAGTTCTCCCCAGGTCAGGGCCTGGGTGGCGGGGTCGAAGC
CCTGCTTGGTGACATAGACCAGGAAGTAGTCGGCGCCGTGGCTGGCCTGG
TCGTGGAGCTTGACGGTGAAGTCGTCGGTGACGTCCGTGGTCTGCCACGG
GCCCACGGCGTCGAGGGAGTTGTAGCGTCCGCTCTCGGTACGGCCGCCGC
TGCACAGCTGGCCGTCGGGGACGACCGCCTCGAAGTCGCCGGCCGAGCCG
TTGCGGTAGAGGCCGTTCCAGTTCCACATGGCGTTGGGGTCGTCCTGCCA
CGCCTGCCAGCACATGGGGTCCTCGTCGGCCATGGCCGGGTTCTGGAAGT
CGTCGCCCCAGCGTTCCCAGCAGCCGTAGTTGCGGGAGGCGGGGTCGACG
ACCGAGCCGTGGGCCACGGCGGTGCCGCTCCAGGGGATCAGGGTGAGCAG
GACCGCGGCCAGAGTGCTGAGGACGAGGGTCGTCGCCCGTCGGGCCCTTC
CGGCGAGTTGAATTTTGGCGCGATCATGACAAGTCAT
Amino acid sequence (underlined, the signal
peptide not present in mature protein) -
>tr|Q9RJC1|Q9RJC1_STRCO Putative secreted
cellulose-binding protein OS = Streptomyces
coelicolor (strain ATCC BAA-471/A3(2)/M145)
GN = SCO0643 PE = 1 SV = 1
SEQ ID NO: 6
MTCHDRAKIQLAGRARRATTLVLSTLAAVLLTLIPWSGTAVAHGSVVDPA
SRNYGCWERWGDDFQNPAMADEDPMCWQAWQDDPNAMWNWNGLYRNGSAG
DFEAVVPDGQLCSGGRTESGRYNSLDAVGPWQTTDVTDDFTVKLHDQASH
GADYFLVYVTKQGFDPATQALTWGELQQVARTGSYGPSQNYEIPVSTSGL
TGRHVVYTIWQASHMDQTYFLCSDVDFG
PcLPMO9D
DNA sequence -
>gi|370987905:23-730 Phanerochaete chrysosporium
gh61D mRNA for glycoside hydrolase family 61
protein D, complete cds
SEQ ID NO: 7
ATGAAGGCCTTCTTCGCCGTTCTCGCTGTCGTCTCTGCGCCATTCGTCCT
GGGACACTACACCTTCCCCGACTTCATTGAGCCTAGCGGGACCGTCACCG
GGGATTGGGTCTATGTCCGCGAGACCCAGAACCACTACAGTAACGGACCG
GTCACCGATGTCACAAGCCCCGAGTTCCGCTGCTACGAGCTGGACCTGCA
GAACACTGCAGGACAGACGCAGACCGCTACGGTTTCCGCCGGTGACACTG
TTGGATTCAAGGCGAACAGCGCAATTTACCACCCAGGATACCTCGACGTC
ATGATGTCCCCGGCCTCCCCAGCCGCCAACTCGCCCGAGGCCGGCACTGG
CCAGACCTGGTTCAAGATCTACGAGGAGAAGCCACAGTTCGAGAATGGCC
AGCTCGTTTTTGATACCACGCAGCAAGAGGTCACGTTCACGATCCCGAAG
AGCCTTCCCAGTGGCCAGTACCTCCTCCGTATCGAGCAGATCGCCCTCCA
CGTCGCCAGCTCCTATGGCGGTGCACAGTTCTACATCGGGTGCGCTCAAC
TCAACGTCGAGAACGGTGGCAACGGCACCCCGGGCCCGCTCGTGTCGATC
CCAGGTGTCTACACCGGCTACGAGCCCGGTATCCTCATCAACATCTACAA
CCTGCCGAAGAACTTCACCGGCTACCCGGCGCCTGGCCCCGCTGTCTGGC
AAGGATGA
Amino acid sequence (underlined, the signal
peptide not present in mature protein) -
>gi|370987906|dbj|BAL43430.1| glycoside hydrolase
family 61 protein D [Phanerochaete chrysosporium]
SEQ ID NO: 8
MKAFFAVLAVVSAPFVLGHYTFPDFIEPSGTVTGDWVYVRETQNHYSNGP
VTDVTSPEFRCYELDLQNTAGQTQTATVSAGDTVGFKANSAIYHPGYLDV
MMSPASPAANSPEAGTGQTWFKIYEEKPQFENGQLVFDTTQQEVTFTIPK
SLPSGQYLLRIEQIALHVASSYGGAQFYIGCAQLNVENGGNGTPGPLVSI
PGVYTGYEPGILINIYNLPKNFTGYPAPGPAVWQG

Preferred aspects according to the invention are as set out in the Examples in which one or more of the parameters or components used in the Examples may be used as preferred features of the methods described hereinbefore.

The invention will now be described by way of the following Examples in which:

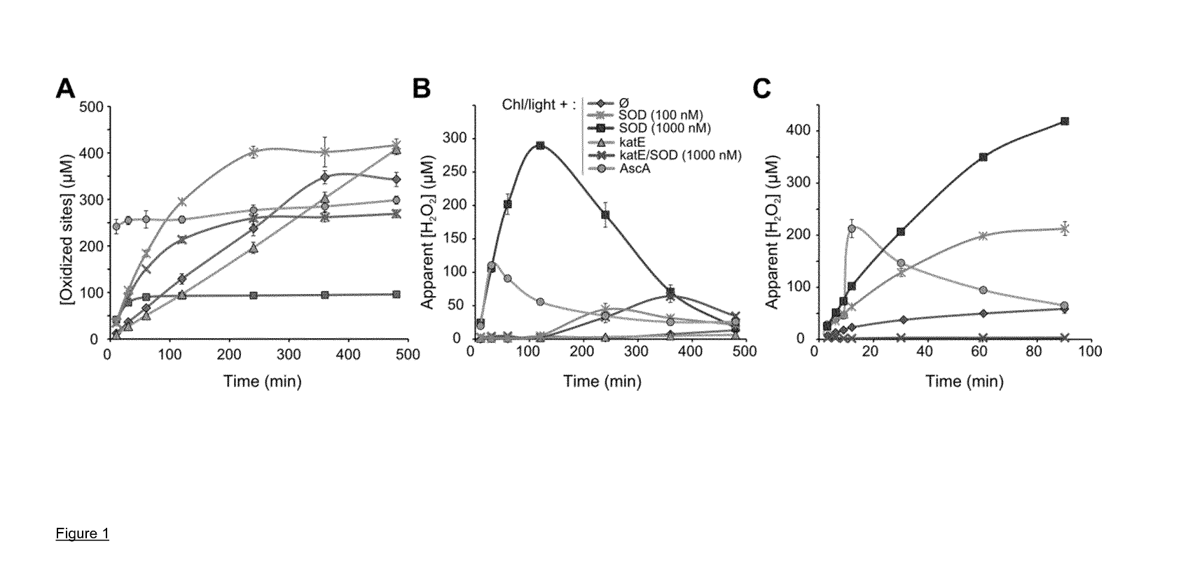

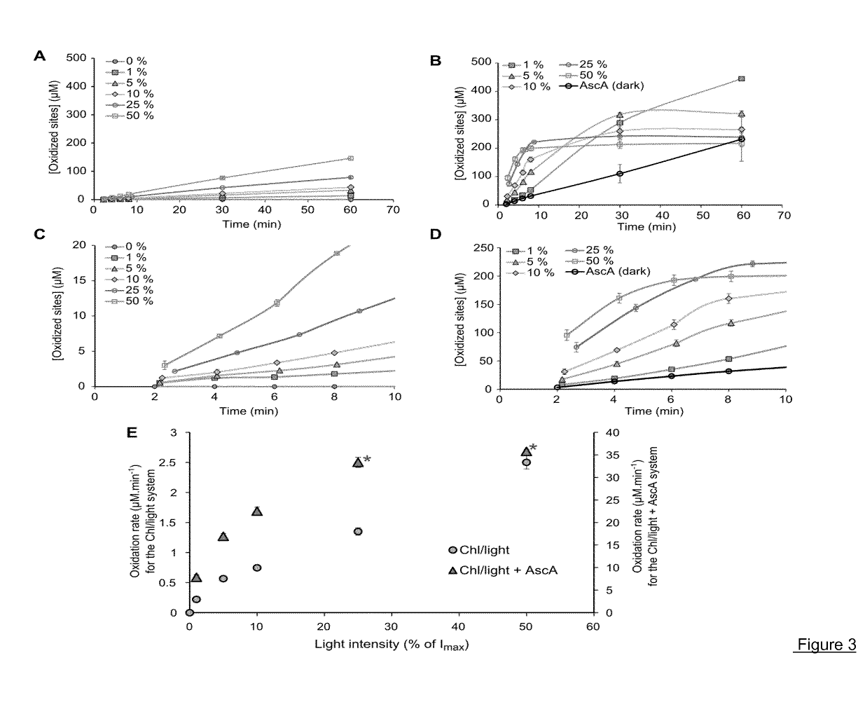

FIG. 1 shows LPMO activity and hydrogen peroxide apparent production when using the Chlorophyllin/light (Chl/light) system for driving the reaction. Panels A and B show time-courses for the release of aldonic acid products (A) and H₂O₂ levels (B) upon incubating Avicel (10 0=¹) with ScLPMO10C (0.5 μM). Reactions were carried out in sodium phosphate buffer (50 mM, pH 7.0) at 40° C., under magnetic stirring, with Chl (500 μM) exposed to visible light (I=25% Iₘₐₓ, approx. 42 W·cm⁻²). Note that, compared to the previous study by Cannella et al. (2016, Nat. Comm., 7, doi:10.1038/ncomms11134), we used higher light intensities, which likely explains why in our hands, the Chl/light system also works in the absence of a reductant (diamonds). Reaction conditions varied in terms of the presence of superoxide dismutase, SOD (100 or 1000 nM), catalase, katE (10 μg·mL⁻¹) or ascorbic acid, AscA (1 mM). Panel C shows the apparent production of H₂O₂ in the absence of ScLPMO10C, with all other conditions being the same as for Panels A & B. Control reaction in the dark did not yield detectable levels of oxidized products (FIG. 3A), with the exception of reactions with AscA (data not shown) The legend code displayed in panel B applies for panels A and C. Error bars show ±s.d. (n=3). Comparison of the panels shows that (a) hydrogen peroxide is produced by various of the LPMO-driving systems (panel C), (b) high production of hydrogen peroxide is associated with high initial LPMO activity (panel A) as well as rapid LPMO inactivation (panel A; progress curves flattening out early in the reaction) and (c) high apparent hydrogen peroxide levels are observed in reactions that show rapid inactivation of the LPMO (panel B).

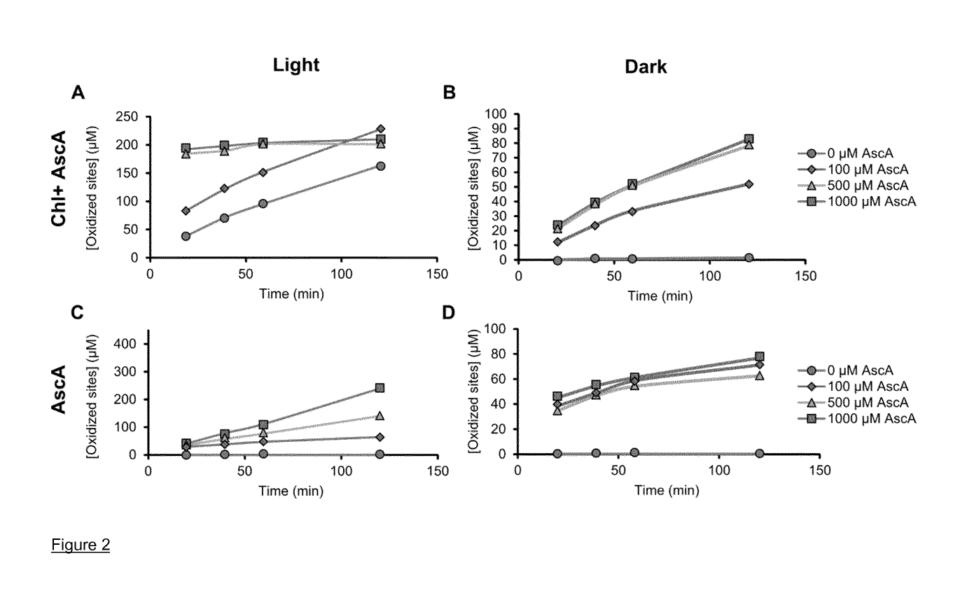

FIG. 2 shows screening experiments to assess the impact of the ratio between Chl and AscA on LPMO activity and stability. The figures show time courses for the release of aldonic acid products from Avicel (10 g·L⁻¹) by ScLPMO10C (0.5 μM) in the light or in the dark and with different doses (0-1000 μM) of AscA, as indicated. In reactions with Chl, Chl was present at a constant concentration of 500 μM. Reactions were carried out in sodium phosphate buffer (50 mM, pH 7.0) at 40° C., under magnetic stirring and exposed to visible light (I=25% Iₘₐₓ, approx. 42 W·cm⁻²) or kept in the dark. Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites.

FIG. 3 shows the effect of light intensity on ScLPMO10C-catalyzed oxidation of Avicel fueled by the Chl/light (A) or Chl/light+AscA (B) systems. Panels (C) and (D) show enlargements of parts of panels (A) and (B), respectively. Panels B and D also show data for a reaction with only AscA. Panel (E) shows the approximate initial oxidation rates (expressed as μM of oxidized sites/min) as a function of light intensity for both systems. Note the different Y-axes; the system with AscA is much faster. For reactions carried out in the presence of AscA and with a light intensity of I=25 or 50% of Iₘₐₓ the reaction was so fast that the rate (derived from the 2 first time points) is probably underestimated (stars). Reactions were carried out in sodium phosphate buffer (50 mM, pH 7.0) at 40° C., under magnetic stirring and exposed to visible light (I=0-50% Iₘₐₓ, approx. 0-84 W·cm⁻²). Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites. Error bars show ±s.d. (n=2). Comparison of the panels shows that LPMO activity, when driven by Chl/light is dependent on the light dose and that the presence of ascorbic acid strongly increases the impact of the Chl/light system, which, as shown below is due to AscA converting produced superoxide to hydrogen peroxide. High initial LPMO activity, likely due to high hydrogen peroxide levels, is associated with rapid inactivation of the LPMO (panel B, curves flattening out early during the time course).

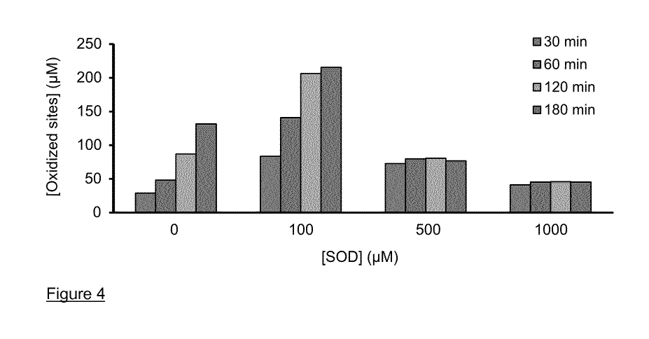

FIG. 4 shows a screening experiment to assess the impact of SOD on ScLPMO10C-catalyzed oxidation of Avicel fueled by the Chl/light system (no AscA). SOD catalyzes the conversion of superoxide, produced by the Chl/light system to hydrogen peroxide and oxygen. The figure shows time courses for the release of aldonic acid products from Avicel (10 g·L⁻¹) by ScLPMO10C (0.5 μM) fueled by Chl (500 μM)/light, in reaction mixtures supplemented with different concentrations of SOD (0-1000 nM). Reactions were carried out in sodium phosphate buffer (50 mM, pH 7.0) at 40° C., under magnetic stirring and exposed to visible light (I=25% Iₘₐₓ, approx. 42 W·cm⁻²). Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites. The figure shows that a low SOD concentration, giving rise to lower hydrogen peroxide concentrations, is benefical for LPMO activity, whereas high SOD concentrations, giving rise to higher hydrogen peroxide concentrations, is detrimental for LPMO activity,

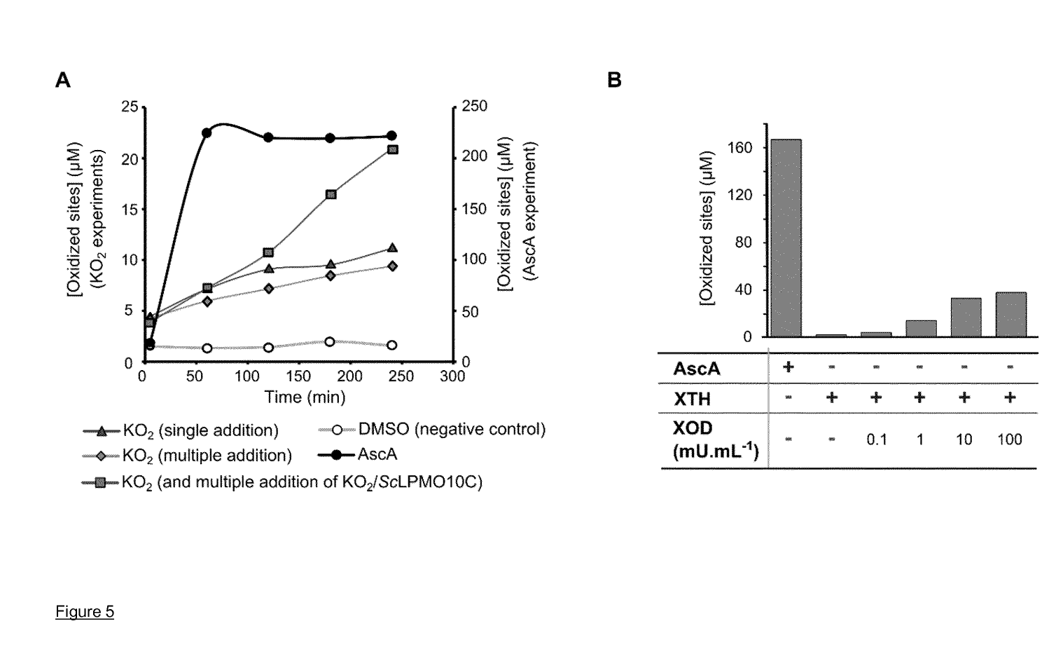

FIG. 5 shows reductive activation of ScLPMO10C by superoxide. (A) Time-course for the release of aldonic acid products from Avicel (10 g·L⁻¹) by ScLPMO10C (1 μM) in Tris-HCl (pH 8.0, 50 mM). Reactions were initiated by the addition of KO₂ (280 μM final concentration, approximately) or AscA (1 mM final concentration). The KO₂ stock solution was prepared in dried and N₂-flushed DMSO. Due to the instability of KO₂ in protic solvents its actual concentration once added in the reaction mixture cannot be controlled. For multiple additions, KO₂ (280 μM final concentration), alone or with and ScLPMO10C (1 μM final concentration) were added after each sampling time. The oxidation rate measured for KO₂-triggered reactions was on average 70-fold lower than for those initiated by AscA (note that the scales of the two y-axes differ by one order of magnitude). Note that superoxide will spontaneously disproportionate to H₂O₂, which can have a negative effect on LPMO activity if the H₂O₂ concentrations become too high; this is illustrated by the beneficial effect of repetitively adding ScLPMO10C. (B) Quantity of soluble aldonic acids released from Avicel (10 g·L⁻¹) by ScLPMO10C (0.5 μM) after 18 h incubation of reactions prepared in sodium phosphate (50 mM, pH 7.0) and fueled by the system xanthine (XTH, 500 μM)/xanthine oxidase (XOD, 0.1 to 100 to mU·mL⁻¹). A control reaction initiated by AscA (500 μM) instead of XTH/XOD was also prepared. All reactions were carried out at 40° C., under magnetic stirring in the dark. Before product quantification, cello-oligosaccharides were hydrolyzed by TfCel5A, yielding oxidized products with a degree of polymerization of 2 and 3 [GlcGlc1A, (Glc)₂Glc1A], summed up to yield the concentration of oxidized sites.

FIG. 6 shows the impact of initial exogenous H₂O₂ on cellulose oxidation efficiency under various conditions. The three panels show time courses for the release of aldonic acid products from Avicel (10 g·L⁻¹) by ScLPMO10C (0.5 μM) fueled by the system Chl (500 μM)/light+AscA (1 mM) (left), only water (middle) or AscA (1 mM) (right), as indicated in the Figure. Reactions were carried out in sodium phosphate buffer (50 mM, pH 7.0) at 40° C., under magnetic stirring and exposed to visible light (I=25% Iₘₐₓ, approx. 42 W·cm⁻²) for Chl/light+AscA reactions or incubated in the dark for the other reactions. Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites. Comparison of the panels shows that (a) hydrogen peroxide alone is not sufficient to drive the LPMO reaction (i.e., a reductant is also needed) (middle panel), (b) Exogenous hydrogen peroxide has no positive effect and, at higher concentrations, a negative effect when applied to the Chl/light system (left panel), likely because this system itself produces large amounts of hydrogen peroxide leading to high LPMO activity and rapid inactivation (FIG. 1), (c) lower concentrations of hydrogen peroxide are beneficial for LPMO activity if AscA is present (right panel).

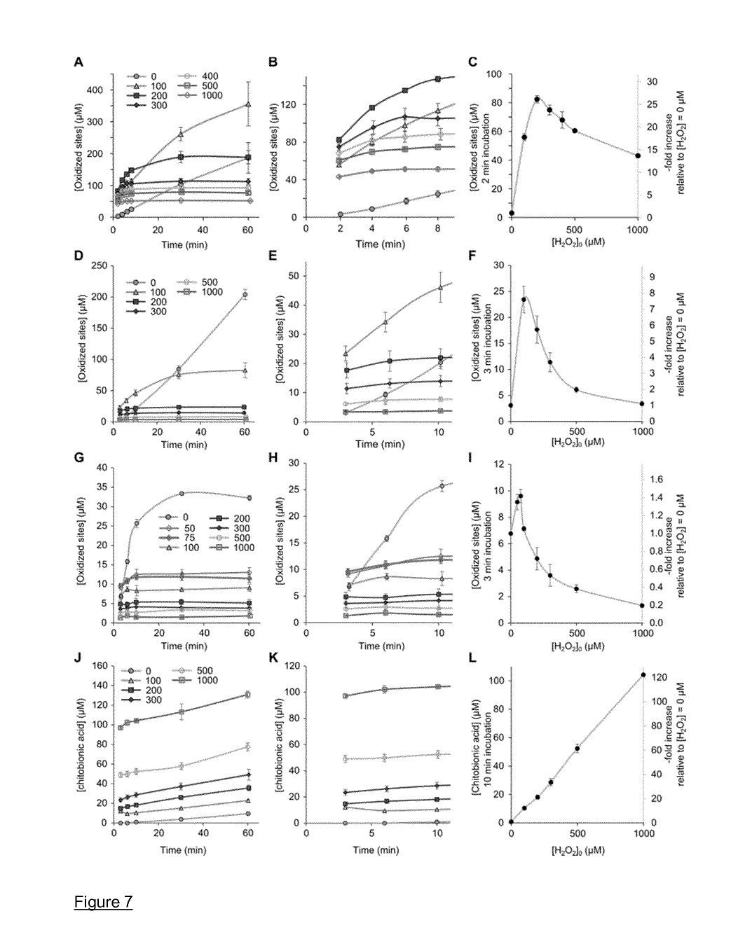

FIG. 7 shows the effect of H₂O₂ on LPMO activity. The panels show time-courses for the release of aldonic acid products from Avicel (10 g·L−1) by 0.5 μM ScLPMO10C (A; zoom-in view in B), 0.5 μM PcLPMO9D (D, E), 0.5 μM ScLPMO10B (G, H) or from β-chitin (10 g·L−1) by 0.5 μM CBP21 (J, K) in the presence of different initial concentrations of exogenous H2O2 (0-1000 μM) and AscA (1 mM). Panels C, F, I and L show how the apparent initial LPMO rate depends on the H₂O₂ concentration for ScLPMO10C, PcLPMO9D, ScLPMO10B and CBP21, respectively. The relative increase in apparent initial rate (calculated on the basis of product formation after 2, 3, 3 and 10 min for ScLPMO10C, PcLPMO9D, ScLPMO10B and CBP21, respectively) compared to the reference reaction (without H₂O₂) is provided on the secondary axis. All reactions were carried out in sodium phosphate buffer (50 mM, pH 7.0) at 40° C., under magnetic stirring, in the dark. Before product quantification, cello-oligosaccharides were hydrolyzed by TfCel5A, yielding oxidized products with a degree of polymerization of 2 and 3 [GlcGlc1A, (Glc)2Glc1A], summed up to yield the concentration of oxidized sites. Solubilized chito-oligosaccharides were hydrolyzed with a chitobiase, yielding chitobionic acid as the only oxidized product. Error bars show ±s.d. (n=3).

FIG. 8 shows the proposed LPMO-guided H₂O₂ splitting mechanism for enzymatic oxidative cleavage of polysaccharides. In the proposed mechanism, LPMO—Cu(II) is first reduced to LPMO—Cu(I) (priming reduction), followed by H₂O₂ binding and homolytic bond cleavage. This cleavage leads to the Fenton-like generation of a hydroxyl radical, catalyzing HAA either from the Cu(II)-bound hydroxyl (haa1) or from the substrate (haa1′). The former scenario would generate a copper-oxyl intermediate that would then abstract a hydrogen from the substrate (haa2). In both scenarios a water molecule is eliminated and attack of the Cu(II)-bound hydroxyl on the substrate radical leads to hydroxylation of the substrate and to regeneration of the Cu(I) center, which can enter a new catalytic cycle. The resulting hydroxylated polysaccharide undergoes molecular rearrangement leading to lactone formation and bond cleavage. The previously assumed equation for the LPMO reaction, which is proven to be incorrect herein, could be written as:

LPMO-Cu(II)+O₂+R—H+2e⁻+2H⁺→LPMO-Cu(II)+H₂O+ROH

or, less commonly

LPMO—Cu(I)+O₂+R—H+2e⁻+2H⁺→LPMO—Cu(I)+H₂O+ROH

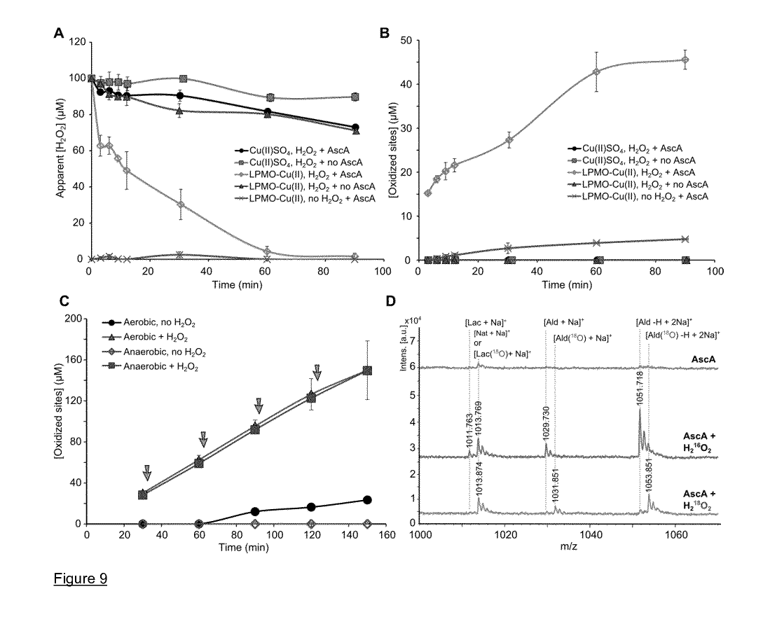

FIG. 9 shows probing the H₂O₂-dependent mechanism of LPMOs. Panels A and B show H₂O₂ consumption (A) and product formation (B) during incubation of ScLPMO10C-Cu(II) and Avicel in the presence or absence of initial exogenous H₂O₂ (100 μM). In control reactions ScLPMO10C-Cu(II) was replaced by Cu(II)SO₄ (0.5 μM). The reaction was initiated by addition of AscA (10 μM) as indicated. Note that this is a very low AscA concentration, meant to test the “priming reduction” hypothesis (see Examples for details). (C) The graphs show time-courses for release of aldonic acid products by ScLPMO10C from Avicel under anaerobic or aerobic conditions at 30° C.; at t=0 and right after sampling every 30 minutes (grey arrows), AscA (to a final added concentration of 10 μM) was added as well as, in the indicated reactions, H₂O₂ (to a final added concentration of 50 μM) (D) MALDI-TOF MS spectra of products obtained after 4 min reaction in the presence of 100 μM H₂¹⁶O₂ or H₂¹⁸O₂, as indicated, and 1 mM AscA. The spectrum shows the DP6 cluster, showing sodium adducts of the native (Nat) hexaose, and the two forms of the oxidized hexaose, the lactone (Lac) and the aldonic acid (Ald). All reactions (panel A to D) were carried out with ScLPMO10C-Cu(II) (0.5 μM) and Avicel (10 g·L⁻¹) in sodium phosphate buffer (50 mM, pH 7.0) at 40° C. under magnetic stirring (unless otherwise stated). The error bars show s.d. (n=3).

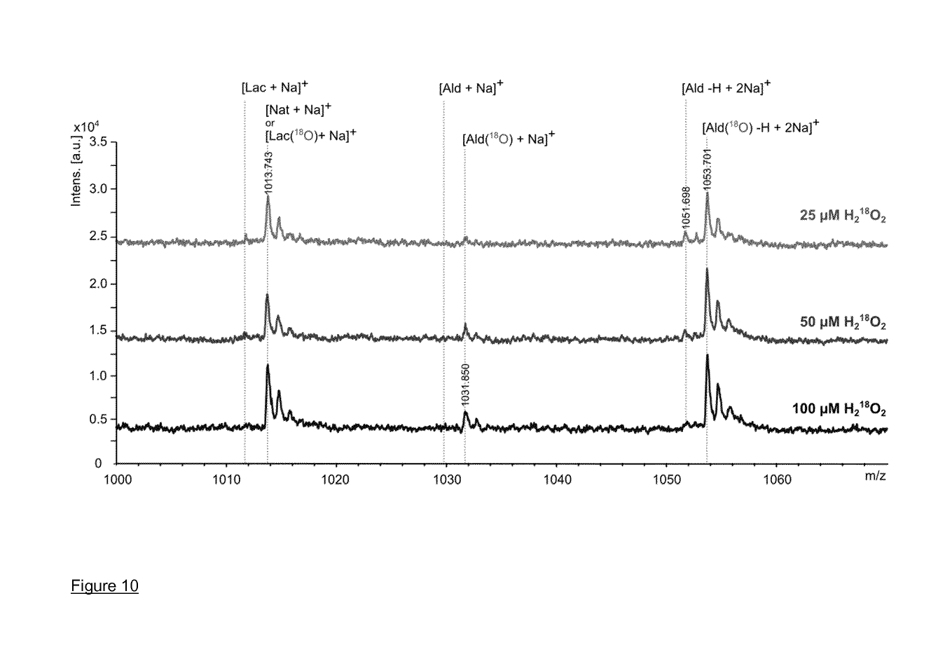

FIG. 10 shows assessment of the competition between H₂O₂ and O₂ by using low concentrations of H₂¹⁸O₂. ScLPMO10C (0.5 μM) was incubated in sodium phosphate buffer (50 mM, pH 7.0) with Avicel (10 g·L⁻¹) at 40° C. under magnetic stirring during 20 min before addition of a “priming” amount of AscA (10 μM) to initiate the reaction. H₂¹⁸O₂ (25, 50 or 100 μM) was added to the reaction mixture just before AscA addition. The graph shows MALDI-TOF MS spectra for the DP6 cluster, showing sodium adducts of the native (Nat), lactone (Lac) and the aldonic acid (Ald) form. The graph shows the product profile obtained after 4 min reaction. Note that under the conditions used here the concentration of (non-labeled) ¹⁶O₂ in solution is in the range of 200-250 μM. Abbreviations: DP, degree of polymerization; Nat, native; Lac, oxidized, lactone form; Ald, oxidized, aldonic acid form. Nb: The H₂¹⁸O₂ solution contains approximately 10% of H₂¹⁶O₂ which might partly explain the small peak observed at m/z=1051.7. Under these conditions, where there is approximately 10 times more ¹⁶O₂ that H₂¹⁸O₂; the fact that, still, the oxygen that ends up in the oxidized products is ¹⁸O provides the strongest possible proof that indeed H₂O₂ is the co-substrate and not O₂.

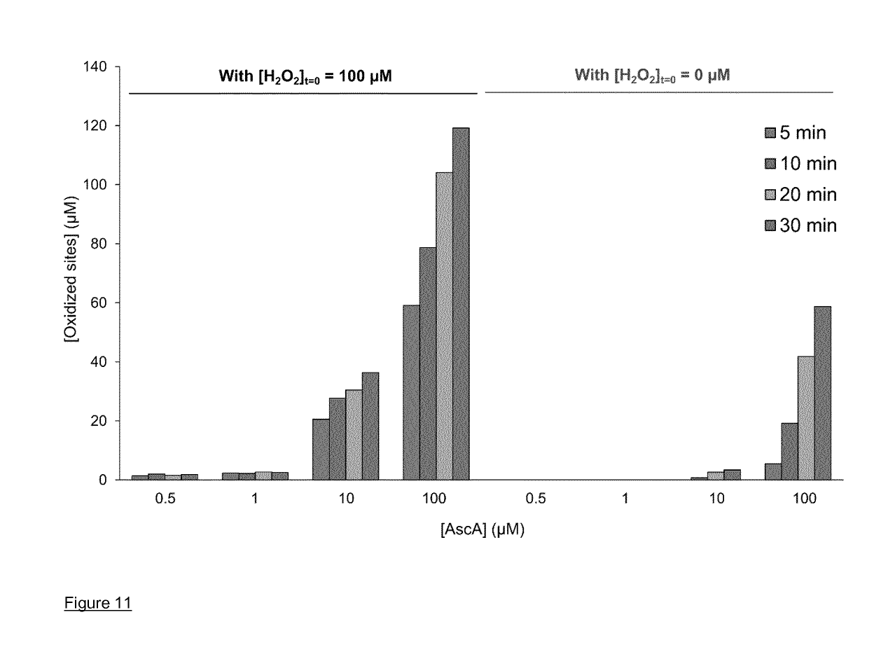

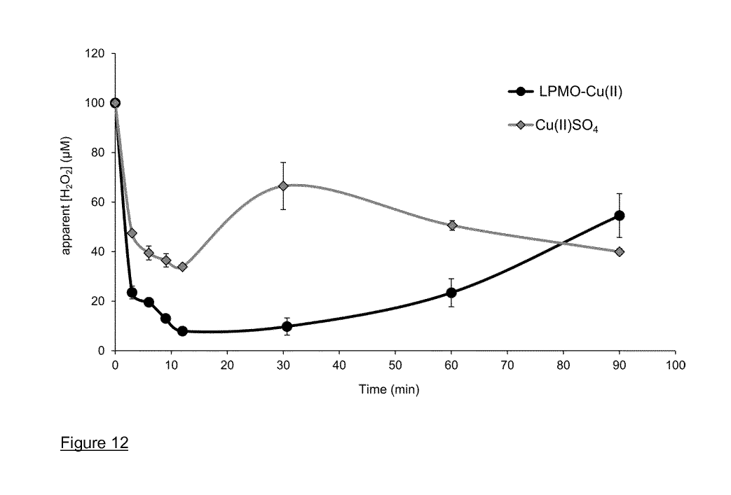

FIG. 11 shows screening of conditions to probe the “priming reduction” hypothesis. The graph shows time-courses for the release of aldonic acid products from Avicel (10 g·L⁻¹) by ScLPMO10C (0.5 μM) in presence (100 μM, left side) or absence (right side) of initial exogenous H₂O₂ and different concentrations of AscA (0.5-100 μM). All reactions were carried out in sodium phosphate buffer (50 mM, pH 7.0) at 40° C., under magnetic stirring, in the dark. Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites. In the absence of H₂O₂, one can barely observe oxidized products during the first 30 min for AscA concentrations between 0.5 and 10 μM. In the presence of H₂O₂, 10 μM of AscA seems to be the lowest concentration allowing detection of soluble oxidized products. Note that the amount of oxidized products detected in the presence of H₂O₂ (left side) is higher than the concentration of added AscA, which confirms that AscA is not required in stoichiometric amounts (as was previously assumed) and thus confirms the priming reduction hypothesis. Note that AscA can produce hydrogen peroxide from molecular oxygen, which explains the activity at 100 μM AscA in the absence of hydrogen peroxide. At high AscA concentrations AscA can further reduce the H₂O₂ to water (FIG. 12).

FIG. 12 shows H₂O₂ consumption at high AscA concentration. The reaction mix contained ScLPMO10C-Cu(II) (0.5 μM), H₂O₂ (100 μM) and Avicel (10 g·L⁻¹) in sodium phosphate buffer (50 mM, pH 7.0). In the control reaction (upper line) ScLPMO10C-Cu(II) was replaced by Cu(II)SO₄ (0.5 μM). The reactions were initiated by addition of ascorbic acid (1 mM). An initial concentration of 100 μM of exogenous H₂O₂ was chosen since at this concentration ScLPMO10C maintains activity during at least the first hour and is not quickly inactivated (Cf FIG. 7). In both reactions the H₂O₂ concentration decreases, which shows that this decrease is not only due to H₂O₂ consumption by the LPMO, but also to reactions with AscA. Error bars show ±s.d. (n=3).

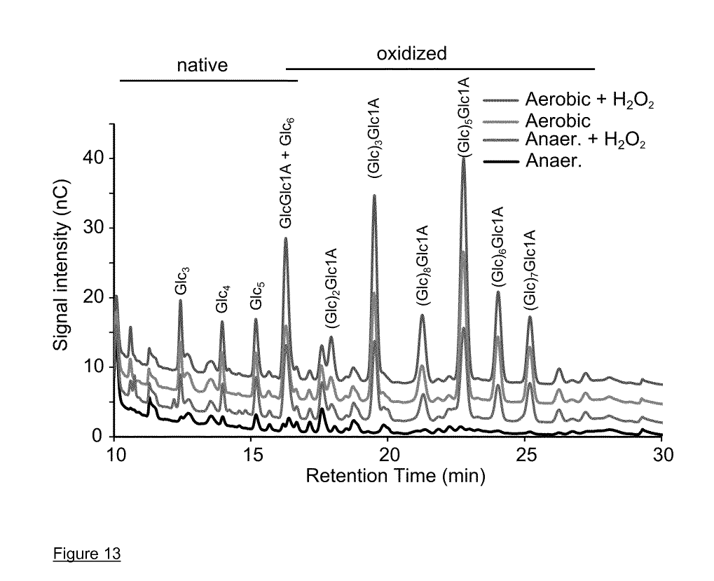

FIG. 13 shows the product profile (HPAEC-PAD) obtained after degradation of cellulose by ScLPMO10C in reactions carried out in aerobic or O₂-free conditions, in presence or absence of initial exogenous H₂O₂ (100 μM). Anaerobic or aerobic solutions of ScLPMO10C-Cu(II) (0.5 μM) and Avicel (10 g·L⁻¹) prepared in sodium phosphate buffer (50 mM, pH 7.0) were incubated at 30° C. under magnetic stirring, supplemented or not with 100 μM H₂O₂. Reactions were initiated by the addition of AscA (1 mM). Each chromatogram is the average of 3 replicates and shows the product profile after 30 min of incubation. The small peaks observed in the anaerobic control reaction (bottom line) correspond to background signals present in the substrate. Sampling at 60 or 90 min (not shown) led to identical chromatograms indicating that the reaction was already over after 30 min. Note that the experimental conditions of this experiment differ from the conditions used in FIG. 9, where lower AscA concentrations were used.

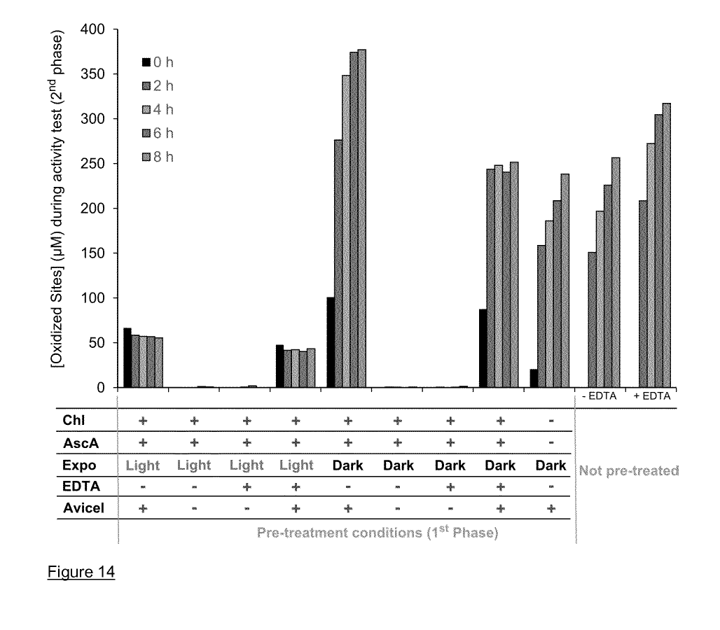

FIG. 14 shows a study of ScLPMO10C inactivation by the Chl/AscA system in the dark or in the light. The experiment consisted of two phases, namely a pretreatment phase followed by an activity test phase. During the first phase ScLPMO10C (0.5 μM) was incubated in sodium phosphate buffer (50 mM, pH 6.0) at 40° C. under magnetic stirring in presence of Chl (500 μM) and AscA (1 mM). Conditions varied in terms of light exposure [visible light, 25% I. (eq. 42 W·cm⁻²), or dark], presence or absence of Avicel (10 g·L⁻¹) and presence or absence of EDTA (0.5 mM; to chelate metals). After 2 h of incubation, half of the pre-treated sample (250 μL) was directly transferred to 245 μL of a pre-incubated (20 min, 40° C.) suspension of Avicel (10 g·L⁻¹) in sodium phosphate buffer (50 mM, pH 6.0). To secure initiation of the second phase reaction AscA was added (5 μL of a 100 mM solution, yielding 1 mM final concentration). The second phase reaction mixtures were incubated in a thermomixer (40° C., 850 rpm) for up to 8 h, after which the release of oxidized products was analyzed. For the second phase, two control reactions were set up containing ScLPMO10C (0.25 μM), in presence (0.25 mM) or absence of EDTA, incubated with Avicel (10 g·L⁻¹) in sodium phosphate buffer (50 mM, pH 6.0) in a thermomixer (40° C., 850 rpm) and initiated by addition of AscA (1 mM). The data show that use of the Chl/light-AscA leads to complete inactivation of the enzyme, regardless of whether the pre-treatment phase had been carried out in the presence or absence of substrate. Conversely, the enzymes that were pre-treated in reactions kept in the dark and in presence of substrate are still able to catalyze cellulose oxidation, whereas enzymes pre-treated in the dark in the absence of substrate are inactivated. This latter observation demonstrates a protective role of the substrate. EDTA does not prevent the inactivation, suggesting that the inactivation mechanism does not involve free metals present in solution. Note that the two reference reactions displayed to the right show that EDTA, at this concentration, does not harm LPMO activity.

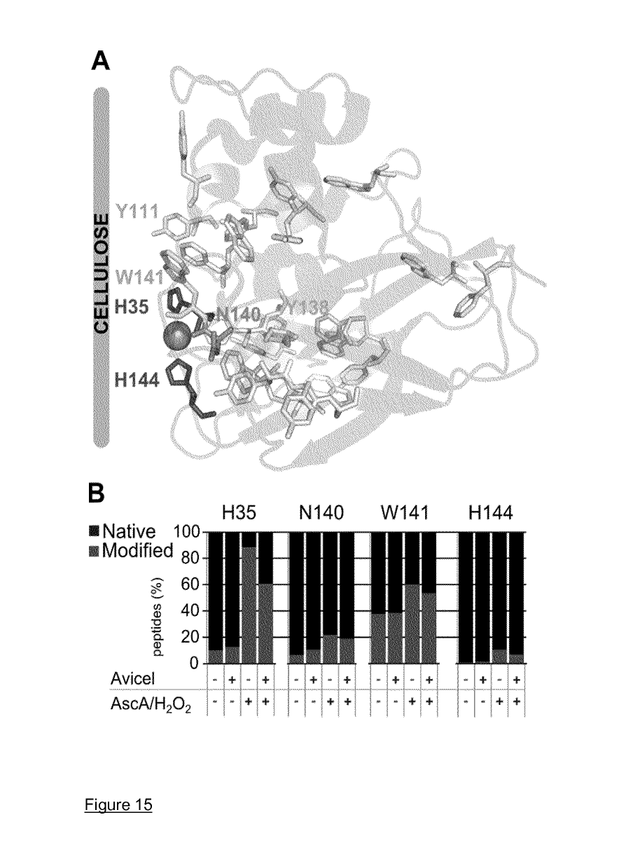

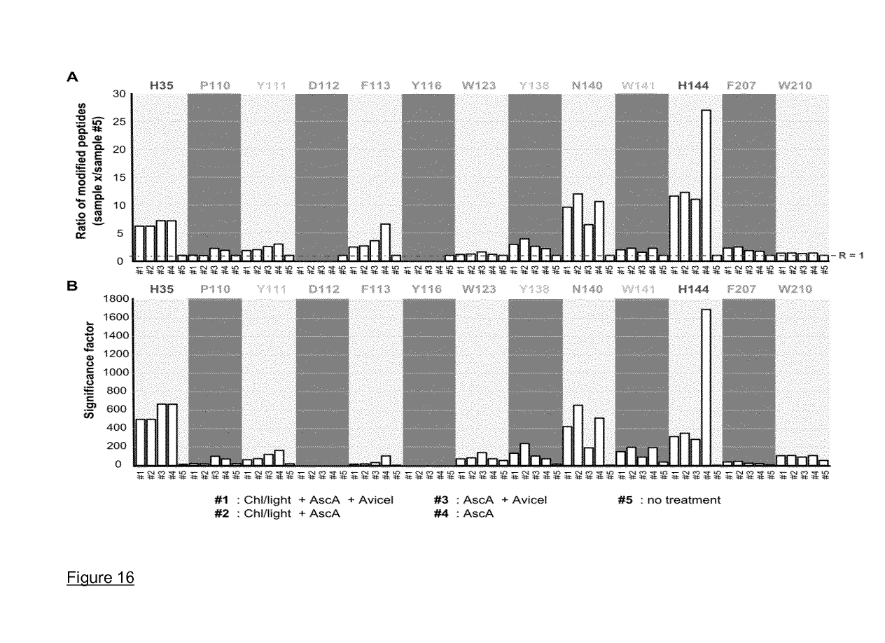

FIG. 15 shows LPMO self-oxidation and the protective role of the substrate. (A) Mapping of the most frequently modified residues on the structure of the catalytic domain of ScLPMO10C (PDB 4OY7) reveals that oxidation occurs in and near the active site, predominantly on the catalytic histidines, H35 and H144. The colour code highlights the degree of oxidation: high (dark grey), middle (grey) and low (light grey). For aromatic residues shown as grey sticks no modification was detected (See FIGS. 16 & 17). (B) Impact of substrate on the ratio of modified/native peptides bearing H35, N140, W141 or H144 after a short incubation. ScLPMO10C (1 μM) was pre-treated by 20 min incubation in sodium phosphate buffer (50 mM, pH 7.0) at 40° C. under magnetic stirring, in the presence (10 g·L⁻¹) or absence of Avicel and addition of either AscA (1 mM)/H₂O₂ (100 μM) or simply water (control reaction). (See FIG. 18 for corresponding activity tests).

FIG. 16 shows the identification of residues modified during LPMO inactivation. (A) Ratio (R) between the fraction of modified peptides in sample x (#1-#4) and the fraction of modified peptides in sample #5 for peptides modified at position y (indicated above the chart). (see Materials and methods section, R=1 for sample #5). (B) Significance factor for modification of the indicated residues in treated samples of ScLPMO10C. The significance factor equals R times the frequency of modification; see Materials and methods section). From this analysis, residues considered as significantly affected are the catalytic histidines, H35 and H144, N140, and, to a lesser extent, Y111, Y138 and W141 (cf FIG. 15A for structural mapping). Pre-treatment conditions were as follows: ScLPMO10C (1 μM, eq. 17.3 μg) was exposed to [Chl/light+AscA] (#1 and #2) or to AscA (#3 and #4), in the presence (#1 and #3) or absence (#2 and #4) of Avicel (10 g·L⁻¹). A control experiment (#5) was carried out in the absence of substrate and electron source. All reactions were incubated during 2 hours in sodium phosphate buffer (50 mM, pH 6.0), under magnetic stirring at 40° C. 500 μM of Chl, 1 mM of AscA and an intensity of 25% Iₘₐₓ (eq. 42 W·cm⁻²) were employed, as indicated.

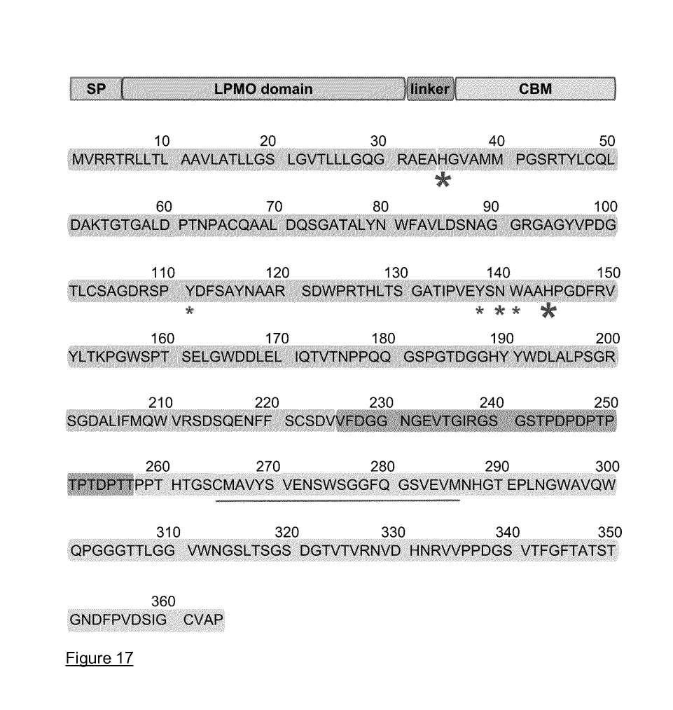

FIG. 17 shows the location of oxidative modifications in ScLPMO10C. The mature protein, used for the study, does not contain the signal peptide (amino acids residues 1-34). The protein is composed of the LPMO domain (aa. 35-225) and a CBM (aa. 258-364), connected by a linker (aa. 226-257). Modifications considered as significant (big or small star; see FIG. 16 and FIG. 15A) occur in the LPMO domain only, whereas the linker and the CBM are not affected. The sequence coverage of all the samples analyzed in this study was in between 78 and 90%. (The sequence underlined represents a non-covered region).

FIG. 18 shows the results of assessing inactivation of ScLPMO10C by AscA/H₂O₂ and the protective role of the substrate. The experiment consisted of two phases, namely a pretreatment phase followed by an activity test phase. During the first phase ScLPMO10C (1 μM) was incubated in sodium phosphate buffer (50 mM, pH 7.0) at 40° C. under magnetic stirring, in the presence (10 g·L⁻¹) or absence of Avicel during 20 min before addition of either AscA (1 mM)/H₂O₂ (100 μM), AscA (1 mM)/H₂¹⁸O₂ (100 μM) or water and further incubation for 20 min. This first phase is the so-called pre-treatment phase. Then, half of the pre-treated sample (250 μL) was directly transferred to 245 μL of a pre-incubated (20 min, 40° C.) suspension of Avicel (10 g·L⁻¹) prepared in sodium phosphate buffer (50 mM, pH 7.0). To ensure activity fresh AscA was also added (5 μL of a 100 mM solution, giving a final concentration of 1 mM). The reactions were incubated in a thermomixer (40° C., 850 rpm) for up to 20 h. The release of oxidized products during this second phase was monitored and is shown in the graphs. Before product quantification, celloligosaccharides were hydrolyzed by TfCel5A, yielding oxidized products with a degree of polymerization of 2 and 3 [GlcGlc1A, (Glc)₂Glc1A], summed up to yield the concentration of oxidized sites. Similar to experiments performed with Chl+AscA (FIG. 14), the data show that pre-treatment with AscA/H₂O₂ in the absence of substrate leads to inactivation of the enzyme. The presence of substrate protects the enzyme and leads to similar initial LPMO rates in the second phase, compared to the control sample (i.e. not exposed to AscA/H₂O₂). The experiment depicted in the Figure was performed to validate the protocol of LPMO inactivation/substrate protection employed for the analysis of LPMO self-oxidation by HPLC-MS/MS (See FIG. 15B). The error bars show s.d. (n=2).

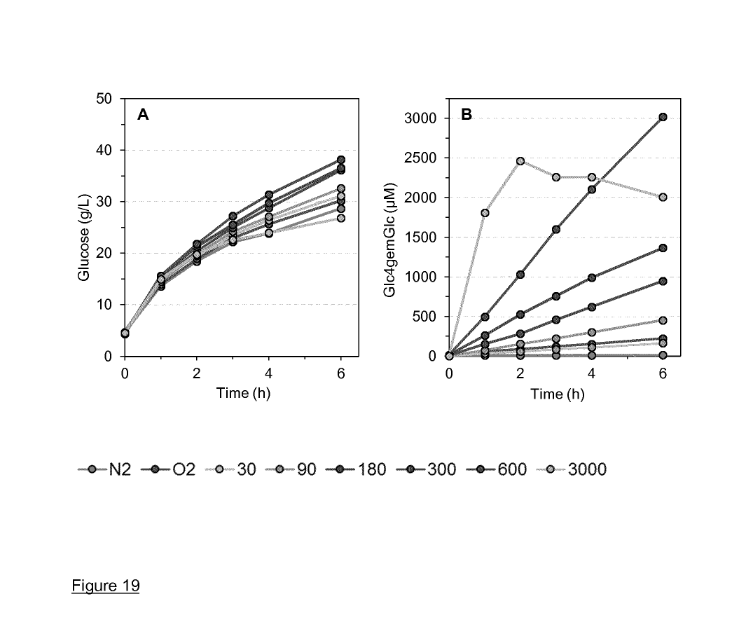

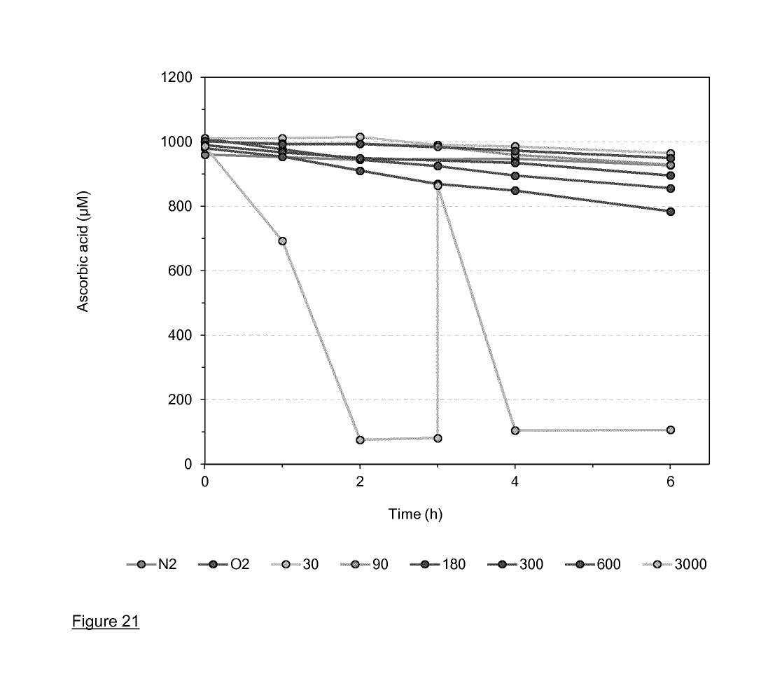

FIG. 19 shows saccharification of Avicel by Cellic® CTec2 (4 mg/g DM) at various H₂O₂feeding rates. Panel A shows production of glucose and panel B shows production of Glc4gemGlc, the 4-ketoform of cellobiose, which is by far the dominating product of LPMO activity under these conditions (all longer products are converted to this short product due to the presence of cellulases). All reaction mixtures contained Avicel at 10% (w/w) DM and 1 mM AscA. H₂O₂ was supplied at a constant flow rate of 600 μL h⁻¹ using H₂O₂ stock solutions with appropriate concentrations (see Table 3 for details), to obtain the desired feed rates (μM h⁻¹). To ensure anaerobic conditions, all reactions were carried out with constant sparging of nitrogen at a flow rate of 100 mL min⁻¹, with exception to the aerobic control reaction (O₂), which was sparged with air at the same flow rate. “N₂” stands for the anaerobic control reaction without any added H₂O₂. In the reaction fed at 3000 μM h⁻¹ more AscA, corresponding to a final concentration of 1 mM, was added at 3 hours (see text and FIG. 21 for more details). Error bars for glucose concentrations represent standard deviations of two technical replicates.

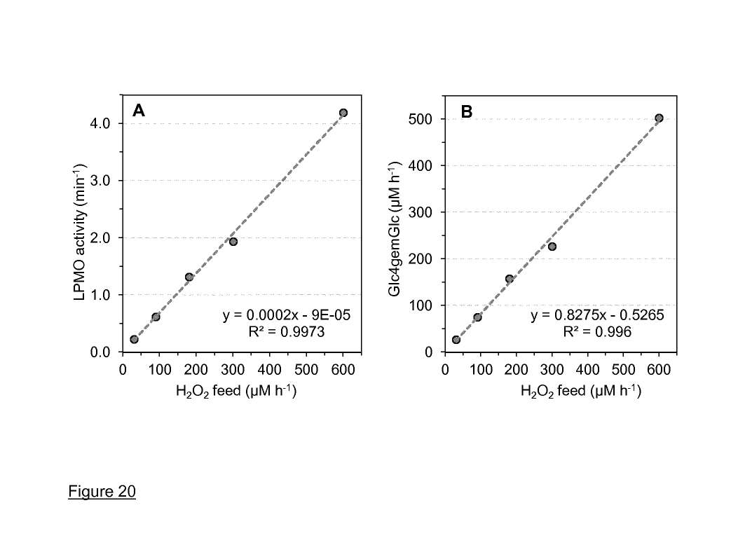

FIG. 20 shows the correlation between the H₂O₂ feeding rate and LPMO activity expressed as an apparent turnover rate (A) and as the formation of Glc4gemGlc (B). Data points are extracted from the experiments depicted in FIG. 19 and Table 3, and represent values calculated after incubating the reaction mixture for 6 h. Note that the rates are slightly underestimated because C1-oxidized products were not quantified.

FIG. 21 shows determination of the AscA concentration during saccharification of Avicel with Cellic® CTec2 at various H₂O₂ feeding rates for a 6 h reaction. In one case, AscA was added during the reaction, with the addition corresponding to a 1 mM final concentration of freshly added AscA. The addition is clearly visible as a spike in the curve. These data refer to the experiments also depicted in FIGS. 19 and 20.

### EXAMPLES

### Example 1

### Materials and Methods

### Materials.

Most of the chemicals were purchased from Sigma-Aldrich. The crystalline cellulose used was Avicel® PH-101 (˜50 μM particles). β-chitin extracted from squid pen was purchased from France Chitin (Orange, France). The superoxide dismutase (SOD) (recombinantly expressed in E. coli, Sigma-Aldrich) was stored (100 μM, eq. 1.63 mg·mL⁻¹) in sodium phosphate buffer (100 mM, pH 7.5), the xanthine oxidase (XOD) (recombinantly expressed in E. coli, Sigma-Aldrich) was stored (2.3 mg·mL⁻¹, eq. 25 U·mL⁻¹) in sodium phosphate buffer (50 mM, pH 7.0). The peroxidase from horseradish (HRP, type II, Sigma-Aldrich) was stored (0.5 mg·mL⁻¹, eq. 100 U·mL⁻¹) in sodium phosphate buffer (50 mM, pH 6.0). The catalase katE from Streptomyces sirex (recombinantly expressed in E. coli) was produced in-house and stored (1.8 mg·mL⁻¹) in Tris-HCl buffer (50 mM, pH 8.0). Ascorbic acid (100 mM) and Amplex Red® (10 mM) stock solutions were prepared in water and DMSO respectively, aliquoted, stored at −20° C. and thawed in the dark for 10 min just before use.

Production and Purification of Recombinant LPMOs.

Recombinant AA10 LPMOs from Streptomyces coelicolor (ScLPMO10C and ScLPMO10B) and from Serratia marcescens (CBP21) were produced and purified according to previously described protocols (Forsberg et al., 2014, Proc. Natl. Acad. Sci. U.S.A, 111, 8446-8451; Vaaje-Kolstad et al., 2005, J. Biol. Chem., 280, 28492-28497). The recombinant fungal AA9 from Phanerochaete chrysosporium K-3 (PcLPMO9D) was produced and purified as previously described (Westereng et al., 2011, PLoS One., 6, e27807). All LPMOs used in this study were prepared in sodium phosphate (50 mM, pH 6.0), copper-saturated with Cu(II)SO₄ and desalted (PD MidiTrap G-25, GE Healthcare) before use (Loose et al., 2014, FEBS Lett., 6-11).

Standard Reaction Conditions.

The reactor was a cylindrical glass vial (1.1 mL) with conical bottom (Thermo Scientific) and the reaction volume was 500 μL. Typical reactions were carried out as follows: the enzyme (0.5 μM) and Avicel (10 g·L⁻¹) were mixed in sodium phosphate buffer (pH 7.0, 50 mM final concentration after all additions) and incubated at 40° C. under magnetic stirring during 20 min. Photobiocatalytic reactions contained chlorophyllin (500 μM, unless stated otherwise) as a light harvester. Then, the reaction was initiated by adding ascorbic acid (to a final concentration of 1 mM, unless stated otherwise), or turning on the light (I=25% Iₘₐₓ, eq. to 42 W·cm⁻², otherwise stated), or both. At regular intervals, 55 μL samples were taken from the reaction mixtures and soluble fractions were immediately separated from the insoluble substrate by filtration using a 96-well filter plate (Millipore) operated with a vacuum manifold. By separating soluble and insoluble fractions, LPMO activity is stopped, as the LPMOs used in this study do not oxidize soluble cello- or chito-oligosaccharides. Filtered samples were frozen (−20° C.) prior to further analysis. Before quantification, solubilized cello-oligosaccharides were hydrolyzed with the endoglucanase Cel5A from Thermobifida fusca (TfCel5A), yielding glucose, cellobiose and oxidized products with a degree of polymerization of 2 and 3 [GlcGlc1A, (Glc)2Glc1A].

Anaerobic experiments. The different reagents of the reaction mix were made anaerobic separately. A suspension (485 μL) of Avicel (10 g·L⁻¹ in final reaction) in sodium phosphate buffer (50 mM, pH 7.0) was flushed with nitrogen gas in a reaction glass vial during 5 min under magnetic stirring. Solutions (200 μL) of AscA (100 mM), H₂O₂ (20 mM), H₂O and ScLPMO10C (50 μM) were submitted to 3 cycles (10 min/2 min) of vacuum/N₂ using a Schlenk line. Similarly, a solution of NaOH (0.5 M, 50 mL) was submitted to 3 cycles (30 min/5 min) of vacuum/N₂. Following this first O₂ removal, all solutions were placed in an anaerobic chamber (Whitley A35 anaerobic workstation) for 16 hours to ensure complete O₂-free conditions (the lids of the vessels were slightly loose, and magnetic stirring was applied for Avicel suspensions). In parallel, similar Avicel suspensions were incubated under magnetic stirring in aerobic conditions. To set-up reactions, ScLPMO10C (0.5 μM final concentration) was then added to anaerobic and aerobic Avicel suspensions. After 20 min incubation, H₂O₂ (100 μM final concentration) was added to half of the reactions (aerobic and anaerobic), whereas water was added to the other reactions. All the reactions (500 μL final volume) were initiated by addition of AscA (1 mM final concentration). The aerobic reactions constitute positive controls ensuring that the treatment of the different solutions (enzyme or AscA) did not harm the integrity of the reactants. 50 μL of each reaction was sampled at regular intervals (usually every 30 min) and mixed with 50 μL of NaOH (0.5 M, aerobic or anaerobic solution) to stop the reaction. All samples were filtrated (as described above) and diluted 2-fold before product analysis by HPAEC-PAD. For quantification purposes, the pH was lowered to pH 6.0 by mixing 40 μL of the sample with 24 μL HCl (0.5 M) before addition of 16 μL of TfCel5A (5 μM in 25 mM Bis-Tris-HCl, pH 6.0, i.e. 1 μM final TfCel5A concentration) and overnight incubation at 37° C. All reactions were performed in triplicate.

Analysis of reaction products. For qualitative analysis, samples were analysed by MALDI-TOF MS, as previously described ((Vaaje-Kolstad et al., 2010, Science, 330, 219-222)). For quantitative analysis, cello-oligosaccharides (native and oxidized) were separated by high performance anion exchange chromatography (HPAEC) and monitored by pulsed amperometric detection (PAD) using a Dionex Bio-LC equipped with a CarboPac PA1 column as previously described (Westereng et al., 2013, J. Chromatogr., 1271(1), 144-152). Chito-oligosaccharides resulting from the action of CBP21 on β-chitin were analyzed by hydrophilic interaction chromatography (HILIC) using a modified version (Loose et al., 2014, FEBS Lett., 588(18), 3435-3440) of a previously described UPLC method (Vaaje-Kolstad et al., 2010, Science, 330, 219-222). The elution of chito-oligosaccharides was monitored using an UV detector (205 nm). Prior to analysis of solubilized mixtures of chito-oligosaccharides, these were hydrolyzed with a chitobiase (1 μM final concentration), yielding chitobionic acid as the only oxidized product. All chromatograms were recorded using Chromeleon 7.0 software.

H₂O₂ measurement. The method is adapted from a previously reported protocol (Kittl et al., 2012, Biotechnol. Biofuels, 5, 79) with some modifications explained hereinafter. For each reaction, 55 μL were sampled at regular intervals and mixed with 55 μL of NaOAc (50 mM, pH 4.5) before filtration (operated with vacuum manifold). Notably, the decrease in pH makes the chlorophyllin insoluble, meaning that it is removed from the solution during the filtration step, providing a transparent and stable filtrate usable for colorimetric determination of H₂O₂ concentration. 30 μL of each filtrate was saved for oxidized product analysis when applicable (cf above). To determine H₂O₂ concentration, 50 μL of the filtrate (or dilutions of it, if necessary) were mixed with 50 μL of a premix composed of HRP (10 U/mL) and Amplex Red (200 μM, 2% DMSO in premix) in sodium phosphate buffer (50 mM, pH 7.5). The reaction mixture (100 μL) was incubated in a 96-well microtiter plate during 10 min before recording the absorbance at 540 nm. For each set of measurements, a blank (sodium phosphate buffer 50 mM, pH 7.0) and H₂O₂ standards (prepared in sodium phosphate buffer 50 mM, pH 7.0) were subjected to the same treatment. Also, an average background control was included to account for the absorbance coming from residual soluble chlorophyllin (small quantities were observed for time points beyond 4 h). To generate this background control, 18 μL of the filtrates from each individual reaction of triplicate chlorophyllin-containing reaction were pooled. 50 μL of this pool (or a dilution equivalent to the one used for the reaction containing Amplex Red®) was mixed with 50 μL of a premix made of HRP (10 U/mL) and DMSO (2% in premix) in sodium phosphate buffer (50 mM, pH 7.5) (i.e. same premix as previously described but without Amplex red). The difference (if any) between this background control and the blank sample was subtracted from the absorbance values of each reaction sample.

LPMO Self-Oxidation: Samples Preparation and HPLC-MS/MS Analysis

To analyze the impact of reaction conditions on protein integrity by HPLC-MS/MS, ScLPMO10C (1 μM, eq. 17 μg in 500 μL total volume) was incubated in sodium phosphate buffer (50 mM, pH 6.0 or 7.0 when stated) in the presence or absence of Avicel (10 g·L⁻¹). The electron providing system was either the Chl (500 μM)/light+AscA (1 mM) system or AscA (1 mM). Control reactions in absence of any exogenous electron source were also run. Reactions were carried out under magnetic stirring at 40° C. during 2 hours or 20 min. Following this pre-treatment phase the reaction was stopped by addition of SDS (4% final) and thermal inactivation (95° C., 15 min). The samples were acidified with 0.1 volume 12% phosphoric acid and proteins precipitated with methanol/Tris as described in the suspension trapping (STrap) protocol (Zougman et al., 2014, Proteomics, 14, 1006-1010). Following trypsin digestion and reversed phase peptide clean-up, the samples were analyzed by HPLC-MS/MS using a nanoLC-QExactive setup (ThermoScientific, Bremen, Germany), with a 140 min reversed phase gradient (0-40% ACN), and a Top10 data dependent acquisition method. The precursor m/z range was set to 300-1500 and the resolution to 70,000 and 35,000 for MS1 and MS2, respectively.

### Data Analysis

The Thermo raw files were converted to mgf format using the msconvert module of the ProteoWizard (v 3.0.9016)(Chambers et al., 2012, Nat. Biotechnol., 30, 918-920). The mgf files were submitted to an error tolerant Mascot (v. 2.4, in-house server) search against a database generated by appending the ScLPMO10C protein to the Uniprot proteome of the expression host, E. coli BL21-DE3.

For a given sample, all the detected peptides were sorted according to their location into the protein sequence (i.e. from N-term to C-term), leading to pool of peptides having globally similar amino acid sequences (with, possibly, small variations due to local modifications). Within a given pool of peptides one can find native peptides (i.e. displaying no modifications at all or only displaying methionine oxidation which commonly occurs during sample processing) and, possibly, modified peptides (i.e. displaying non-typical modifications). Within each pool, the peptides were sub-grouped (via Excel functions) according to the position bearing the modification and sorted by mass changes within each sub-group. This means that, for example, all peptides bearing position H35, native or modified, were sub-grouped together. From those re-arranged data, the a modification frequency could be calculated for each individual residue in the LPMO: F=(number of peptides with modification at position y)/(number of peptides containing position y). Notably, this value has some limits since for modified peptides that are abundant in both the treated (Tre) and the non-treated (reference, Ref) sample, the modification frequency becomes relatively high but not significant in terms of treatment-induced effect. Therefore, the data were normalized by comparing the fraction of modified peptides at position y found in a treated sample, Tre, with the fraction of modified peptides at position y found in the reference sample: R=F^{sample Tre}/F^{sample Ref}. Notably, R could be high in cases where the absolute proportion of modified-to-native peptide is low (relatively to other positions in the peptide sequence). Thus, to identify important (frequent) modifications, both F and R values have to be considered. Important treatment-induced changes will translate into a high fraction of modified peptides (F) within a single sample as well as a high R value. The opposite outcome is expected for a non-significant modification. This “and” condition is mathematically translated by a multiplication leading to a significance factor=F× R.

To analyze the distribution of modifications for a given type of amino acid the number of modified peptides (for a specific modification) was summed up for all residues of the same kind (e.g. H35 and H144 or W123, W141 and W210). This was performed for all the samples. Such statistical distribution analysis allowed to decide, in the light of literature reports, which predicted modifications are relevant or not for a given amino acid. The main modifications that were kept are m/z=−22, −23 and +16 for histidines, +4, +14, +16, +20, +30, +32 and +48 for tryptophans, +16, +30, +32 and +48 for tyrosines, +16 and +32 for phenylalanines, +16 for aspartate, +49 for asparagine and +14, +16 and +32 for proline. The cleaned pool of modified peptides was used to calculate the F and R values.

### Results

When using the Chl/light-AscA system, with relatively high light intensities, a strong increase in LPMO activity was observed, accompanied by an almost immediate inactivation of the enzyme (FIG. 1A). Since light-exposed chlorophyll produces superoxide (O₂^{•-}), we investigated whether addition of superoxide dismutase (SOD) or superoxide-consuming chemicals would allow better control of the reaction, and this turned out not to be the case (data not shown). It is believed that in the particular system investigated enzyme activation occurred too quickly to be controlled. On the other hand, we found that reaction kinetics, reflecting both the catalytic rate and the inactivation of the enzyme, could be modulated by varying the amount of ascorbic acid (FIG. 1A; FIG. 2) or the light intensity (FIG. 3), highlighting the interplay between the various entities. Under these conditions, the Chl/light system (without AscA) also yielded good activity, with less rapid inactivation (FIG. 1A). When using the Chl/light system, a low concentration of SOD (100 nM) was beneficial for LPMO activity without compromising LPMO stability, whereas high concentrations of SOD (1000 nM) were detrimental due to immediate inactivation of the enzyme (FIG. 1A & 4), showing that the levels of superoxide and/or the product of SOD, H₂O₂, affect LPMO activity. It is apparent from these experiments that generation of LPMO co-substrate and LPMO inactivation takes place simultaneously. If the SOD concentration is low, it contributes to LPMO activity by generating hydrogen peroxide which acts as co-substrate, but if its concentration is too high too much hydrogen peroxide is generated which leads to inactivation of LPMO.

A series of reactions were carried out with the Chl/light system, using various combinations of ROS-acting enzymes and monitoring both LPMO activity and H₂O₂ levels (FIG. 1). Both SOD-containing reactions yielded higher initial rates compared to the control reaction. In both reactions, enzyme inactivation was observed. Inactivation happened much faster in the reaction with the higher SOD concentration, and this increased inactivation rate was associated with high levels of H₂O₂ being generated (FIG. 1B; in the control reaction accumulation of H₂O₂ did not take place). Adding a catalase, leading to removal of excess H₂O₂ (FIG. 1C), in the reaction containing 1000 nM SOD indeed led to reduced H₂O₂ levels and reduced inactivation of the LPMO (FIG. 1A,B). The catalase on its own had little effect relative to the control reaction, likely because in this case hydrogen peroxide concentrations were much lower than the Kₘ of the catalase. Addition of AscA, which gives high activity and fast inactivation (see above and FIG. 1A), also led to accumulation of H₂O₂. Reactions in the absence of the LPMO (FIG. 1C) showed that the Chl/light system produces H₂O₂ and that production is strongly increased by adding SOD or AscA. Importantly, addition of AscA led to a fast and drastic increase in H₂O₂ production by the Chl/light system (up to 200 μM within the 12 first minutes of the reaction; FIG. 1C). Control reactions with only AscA yielded more modest H₂O₂ levels (<40 μM within the first 60 min, data not shown), which is very likely related to AscA being less capable of engaging in the thermodynamically challenging reduction of O₂, compared to Chl/light. However, the same overall trend stood out: both higher LPMO activity and faster LPMO inactivation are correlated with higher H₂O₂ levels.

Finally, a key observation is that H₂O₂ production by the Chl/light system (FIG. 1C) cannot be detected in the presence of ScLPMO10C and Avicel (FIG. 1B), a condition that, furthermore, displays quite stable reaction kinetics (FIG. 1A). Altogether these observations led to the hypothesis that H₂O₂ is an unsuspected co-substrate for LPMOs.

Reactions with the Chl/light system (i.e. in the absence of AscA) seemingly lack a reductant needed to reduce the LPMO copper, leading us to speculate that O₂^{•-} could be involved in LPMO reduction. Indeed, chemical (KO₂) or enzymatic (xanthine/xanthine oxidase) O₂^{•-} generation systems could drive LPMO activity, albeit at low levels (FIG. 5). Control experiments carried out in the absence of any electron source but with exogenous H₂O₂ did not lead to cellulose oxidation (FIG. 6), demonstrating that H₂O₂ in itself is not sufficient and that its precursor, O₂^{•-}, is required for LPMO reduction. The first panel of FIG. 6 shows that further addition of H₂O₂ decreases the level of oxidized product (i.e. LPMO activity) which is evidence that the system is already saturated with H₂O₂. In the AscA-fueled system (third panel of FIG. 6), the fact that the addition of H₂O₂ increases or decreases the production of oxidized products when used at different concentrations shows that inactivation and activation of the LPMO is a direct consequence of the level of H₂O₂.

To prove the role of H₂O₂, we then analyzed initial LPMO rates in the presence of varying concentrations of exogenous H₂O₂ (FIG. 7). A spectacular increase in initial LPMO rates was observed at the lower H₂O₂ concentrations, with up to 26-fold more soluble oxidized products being released from Avicel by ScLPMO10C after 2 min when incubated in the presence of 200 μM H₂O₂ (FIG. 7C). This increase in activity is in the same order of magnitude as the increases shown for the Chl/light+AscA system (FIG. 3E). Control reactions in which the enzyme was replaced by Cu(II)SO₄ did not show any oxidized products ruling out any kind of non-enzymatic Fenton chemistry (data not shown). At higher H₂O₂ concentrations, rapid enzyme inactivation was observed. The effect of exogenous H₂O₂ on the activity of fungal LPMO9D from Phanerochaete chrysosporium K-3 (PcLPMO9D) (FIG. 7D-F) and ScLPMO10B (FIG. 7G-I), as well as chitin-active SmLPMO10A, also known in the field as CBP21, showed similar trends, but, quantitatively, the various LPMOs showed differences. This shows that the role of H₂O₂ applies to LPMO generally, but that optimal conditions, such as the concentration of H₂O₂ may vary between LPMOs.

The results described above suggest a catalytic mechanism in which an H₂O₂-derived oxygen atom, rather than an O₂-derived oxygen atom, would be introduced into the polysaccharide chain. In the proposed mechanism (FIG. 8), a priming reduction of the LPMO—Cu(II) to LPMO—Cu(I) occurs first. H₂O₂ would then bind to the Cu(I) center and homolytic bond cleavage, similar to what happens during Fenton chemistry, would produce a hydroxyl radical. This likely leads to formation of a Cu(II)-bound hydroxyl intermediate and substrate radical by one of several possible pathways. In each of these mechanisms, the reaction between a copper-hydroxyl intermediate and the substrate radical leads to hydroxylation of the substrate and to regeneration of the Cu(I) center, which can enter a new catalytic cycle. The resulting hydroxylated polysaccharide undergoes molecular rearrangement leading to formation of an oxidized sugar (chain end) and bond cleavage (Beeson et al., 2012, J. Am. Chem. Soc., 134, 890-892).

To obtain final proof of H₂O₂ being the preferred co-substrate of LPMOs, additional experiments were carried out (FIG. 9). FIGS. 9A&B show that LPMO-dependent consumption of H₂O₂ (FIG. 9A) correlates with the release of oxidized products (FIG. 9B). Importantly, these experiments were done using catalytic (rather than putatively stoichiometric) amounts of reductant (10 μM; i.e. 100 times lower than commonly used concentrations; FIG. 11) to assess the concept of a “priming reduction” and to reduce the effect of AscA on H₂O₂ stability (FIG. 12). (The fact that H₂O₂ concentration also decreases when the LPMO is replaced with Cu(II)SO₄, albeit at a lower speed relative to the reaction catalyzed by the LPMO, shows that H₂O₂ consumption is not only LPMO-dependent and is also due to reaction with AscA).

FIG. 9B shows that product levels are much higher than the total amount of AscA added, in agreement with the proposed mechanism in which, once activated by a priming reduction, a single LPMO can catalyze several reactions provided that the co-substrate, H₂O₂, is supplied.

As a consequence of the above findings, LPMOs should be able to work under anaerobic conditions and this was indeed observed (FIG. 9C; FIG. 13). It is evident from FIG. 13 that anaerobic conditions were achieved as no oxidized products (resulting from O₂) were detected even at high AscA concentrations in the absence of H₂O₂. It is evident that O₂ is not required as oxidized products were produced in anaerobic conditions when H₂O₂ was added. FIG. 9C shows that by adding H₂O₂ and reducing equivalents gradually to the reaction mixture stable kinetics are obtained, with rates that are independent of the presence of O₂. Finally, experiments with a labeled co-substrate, H₂¹⁸O₂, showed that indeed, the oxygen introduced into the polysaccharide chain comes from H₂O₂ and not from O₂ (FIG. 9D and additional data not shown). For example, FIG. 9D shows that when using H₂¹⁸O₂, the characteristic peaks for sodium adducts of the aldonic acid form of an oxidized cellohexaose (m/z 1029.7 & 1051.7) shift by +2 Da. Similar observations were made for the chitin-active AA10 CBP21 (data not shown), as well as a fungal cellulose-active AA9 (data not shown). Reactions with lower concentrations of H₂¹⁸O₂ show that even in the presence of a 10-fold surplus of ¹⁶O₂, oxidized products carry ¹⁸O (FIG. 10).

Several of the reaction progress curves discussed above show that LPMOs are readily inactivated and under some conditions, such as the Chl/light-AscA system (FIG. 1) inactivation seems really fast. Enzyme inactivation was confirmed by a series of experiments where the LPMO was pre-incubated and then tested for remaining activity (FIG. 14). Enzyme inactivation was similar in the presence of EDTA, showing that it is not due to free metal-catalyzed generation of ROS (FIG. 14). Importantly, however, inactivation was partly avoided by the presence of substrate (FIG. 14). Using proteomics technologies, we found that the inactivated LPMO had undergone several oxidative modifications (FIGS. 15 and 16). These modifications were confined to the catalytic histidines and, to a lesser extent, neighbouring residues (Y111, Y138 and W141) (FIG. 15 and additional data not shown). Other residues prone to oxidative damage, such as surface exposed residues in the LPMO domain, the linker or the CBM were not modified (FIG. 17), leading to the important conclusion that oxidative damage is not caused by ROS in solution, as has been suggested (Scott et al., 2016, Biotechnol. Lett., 38, 425-434), but by ROS generated in the catalytic center, i.e. in situ, by the enzyme itself. The protective effect of the substrate (FIG. 14, 18), was reflected in reduced oxidative damage of the N-terminal catalytic histidine (FIG. 15B).

### CONCLUSIONS

The present findings unequivocally show that H₂O₂, and not O₂, is the preferred co-substrate of LPMOs. Basically, LPMOs, after a priming reduction, carry out Fenton-type chemistry (redox-metal driven generation of hydroxyl radicals) in a controlled and substrate-associated manner.

As to the level of H₂O₂ under reaction conditions, it is important to note that, notwithstanding the current findings, LPMOs are able to activate molecular oxygen, albeit at low rate (Kjaergaard et al., 2014, Proc. Natl. Acad. Sci. U.S.A 111, 8797-8802; Kittl et al., 2012, Biotechnol. Biofuels. 5, 79). It is well known that LPMOs generate H₂O₂ in the absence of substrate, which leads to the remarkable conclusion that LPMOs can generate their own co-substrate from O₂. This property may in fact have biological implications; several LPMOs bind weakly to their substrates, meaning that H₂O₂ may be generated by an unbound population, while the bound population uses the H₂O₂ to degrade the substrate. It is likely that substrate-bound, reduced LPMOs bind H₂O₂ with higher affinity than LPMOs in solution, which would explain why low concentrations of exogenous H₂O₂ are beneficial for activity, whereas higher concentrations lead to self-destructive reactions on unbound enzymes. The fact that H₂O₂ production by LPMOs is not observed in the presence of substrate is obviously also due to H₂O₂ being the substrate of the enzyme. Notably, the assumption that substrate-affinity has an impact on H₂O₂ management and self-destruction by the LPMOs sheds new light on the role of the CBMs that are appended to some LPMOs, including ScLPMO10C.

The link between H₂O₂, Fenton-type systems and enzymatic biomass depolymerization has been a matter of debate, controversy and investigations for several decades. The present findings reveal a novel role for H₂O₂ with far reaching implications for the design of biorefining processes. We show here that LPMO performance and stability can be controlled by controlling the supply of H₂O₂, a liquid, easy-to-handle co-substrate. We further show that LPMOs can act in the presence of only catalytic amounts of reductant, which abolishes reductant-induced undesirable redox side reactions, and in the absence of molecular oxygen, abolishing the need for aeration. Notably, overdosing LMPOs can be a problem, since lack of sufficient substrate (i.e. LPMO binding sites on the substrate) may lead to LPMO inactivation. Careful balancing of LPMOs and hydrolytic enzymes (e.g. cellulases) may be necessary for obtaining optimal process conditions, with the cellulases “peeling off” LPMO-disrupted polymer chains from the substrate surface, thus exposing novel LPMO binding sites. As to LPMO stability, it is interesting to note that one of the residues most vulnerable to oxidation, the N-terminal catalytic histidine, is methylated in fungal LPMOs; perhaps this methylation helps protecting the fungal LPMOs from oxidative self-destruction.

### Example 2

### Materials and Methods

### Substrates, Enzymes and Reagents

As cellulosic substrates, Avicel® PH-101 (˜50 μM particles; Sigma Aldrich, St. Louis, USA), sulfite pretreated Norway spruce (Chylenksi et al., 2017, J Biotechnol. 246:16-23) and steam exploded birch (SEB) (Müller et al., 2015, Biotechnology for Biofuels, 8, 187) were used. Lignocellulosic substrates were processed and pretreated as described previously (Müller et al., 2015, Biotechnology for Biofuels, 8, 187; Chylenksi et al., 2017, J Biotechnol. 246:16-23) and had the following compositions (% DM): 88.3% and 43.9% cellulose, 9.3% and 11.6% hemicellulose, 3.8% and 36.5% lignin, for Norway spruce and SEB, respectively.

The commercial cellulase cocktail Cellic® CTec2 was kindly provided by Novozymes NS (Bagsværd, Denmark). The protein concentration was determined with Bio-Rad Protein Assay (Bio-Rad, USA) based on the Bradford method (Bradford, 1976), using Bovine Serum Albumin (BSA) as a standard.

Unless otherwise stated all chemicals were purchased from Sigma-Aldrich and were at least of reagent grade. A hydrogen peroxide solution (30% v/v) was purchased from Merck Millipore (107209, Merck Millipore, Darmstadt, Germany) and diluted in ultrapure water (Merck Millipore) where needed. Stock solutions of reducing agents were prepared in ultrapure water, stored in the dark at −20° C. and thawed in the dark on ice shortly before use.

### Saccharification in Bottles

Avicel (10% w/w DM) was hydrolyzed with Cellic® CTec2 (4 mg protein/g DM) in sodium acetate buffer (50 mM, pH 5.0) using a working volume of 20 mL in 50 mL rubber sealed glass bottles (Wheaton, Millville, USA), that were incubated at 50° C. with shaking at 180 rpm (HT Ecotron, Infors AG). Reactions were carried out with different oxygen concentrations in the headspace (0%, 21%, 50% and 100% v/v O₂). To obtain desired conditions, bottles containing a suspension of substrate in buffer were sparged with a mixture of nitrogen (N₂) and oxygen (O₂) gas at a flow rate of 800 mL min⁻¹ for 5 min, as follows: for 0% O₂, 800 mL min⁻¹ N₂ and 0 mL min⁻¹O₂; for 21% O₂, 632 mL min⁻¹ N₂ and 168 mL min⁻¹ O₂; for 50% O₂, 400 mL min⁻¹ N₂ and 400 mL min⁻¹ O₂; for 100% O₂, 0 mL min⁻¹ N₂ and 800 mL min⁻¹ O₂. After pre-incubation of the bottles for 40 min, reactions were initiated by addition of enzymes with or without an electron donor and H₂O₂, injected sequentially through the rubber septum.

Reductants were provided to reach the following final concentrations: 0.1 mM, 1 mM, 5 mM or 10 mM ascorbic acid; 1 mM gallic acid, 1 mM catechin, 1 mM dithiothreitol; H₂O₂was added to a final concentration of 0.2 mM (the maximum total volume added to the 20 mL reaction mixtures was 0.4 mL). In some reactions, H₂O₂ (0.2 mM) or ascorbic acid (0.1 mM) or both (0.2 mM and 0.1 mM) were added multiple times. Samples (130 μL) were taken at regular intervals and enzymes were immediately inactivated by incubating at 100° C. for 15 min. Samples were centrifuged at 4° C. and 14 000 rpm for 10 min (Centrifuge 5415R, Eppendorf, Westbury, USA). The supernatant was then filtered using a 96-well filter (0.45 μm) plate (Merck Millipore) and stored at −20° C. until further use.

### Saccharification in Bioreactors

Controlled saccharification with continuous feed of H₂O₂ was conducted in 3 L bioreactors (Applikon, Schiedam, Netherlands) with 900 mL working volume, 10% (w/w DM) of cellulosic substrates and Cellic® CTec2 (4 mg/g DM for Avicel and sulfite-pulped Norway spruce and 2 mg/g DM for less cellulose-rich SEB). Reactions were conducted in sodium acetate buffer (50 mM, pH 5.0) at 50° C. To adjust the pH to 5.0 in SEB hydrolysis, 1 mL of 1 M NaOH per g DM of substrate was added. The reactions with Avicel and Norway spruce contained 1 mM of ascorbic acid. The Avicel degradation reactions were pre-incubated with mixing at 350 rpm, until the temperature stabilized at 50° C., after which the mixing speed was reduced to 300 rpm. Similarly, reactions with lignocellulosic substrates were pre-incubated with a mixing at 500 rpm until stable conditions were reached, after which mixing was reduced to 400 rpm. Saccharification was carried out either aerobically or anaerobically. Aerobic conditions were provided by constant sparging of reaction slurry with air at 100 mL min⁻¹, whereas anaerobic conditions were maintained by sparging with N₂ at 100 mL min⁻¹. This sparging was also applied during the pre-incubation step. H₂O₂ was delivered by continuous feeding using a Masterflex L/S Standard Digital peristaltic pump (Cole-Parmer, Vernon Hills, USA) operated at a constant flow rate (600 μL h⁻¹). Unless otherwise stated, the H₂O₂ feed rate was in the range of 30 to 3000 μM h⁻¹; variation in the feed rate was obtained by using different feed solutions, where H₂O₂ had been diluted in ultrapure water. For the lignocellulosic substrates, H₂O₂ feeding was started 30 min after initiation of the reaction. This was done to avoid high local concentrations of H₂O₂ since the biomass was not well mixed initially, but this changed rapidly as the enzymes' action reduced the viscosity. 1 mL samples were regularly withdrawn from the bioreactor. In case of Avicel hydrolysis, 250 μL of the sample was immediately filtered through 0.45 μm using a 96-well filter plate (Merck Millipore) and the filtratewas used for determination of the ascorbic acid concentration. Samples were heat inactivated by incubation at 100° C. for 15 min and stored at −20° C. until further use.

HPLC Analysis of Released Sugars and Measurement of Ascorbic Acid

Glucose released during saccharification of Avicel and lignocelluloses was analyzed by HPLC utilizing a Dionex Ultimate 3000 (Dionex, Sunnyvale, USA) coupled to a refractive index (RI) detector 101 (Shodex, Japan). Hydrolysis products generated from Avicel were separated at 85° C., with 5 mM H₂SO₄ as the mobile phase at 1 mL min⁻¹ flow rate, using a Rezex RFQ—Fast Acid H⁺ (8%) 100×7.8 mm analytical column (Phenomenex, Torrance, USA). Hydrolysis products released from Norway spruce and SEB, were separated using a Rezex ROA-organic acid H⁺ (8%), 300×7.8 mm analytical column (Phenomenex), operated at 65° C. and 0.6 mL min⁻¹ of 5 mM H₂SO₄. Glc4gemGlc was quantified by HPAEC using a Dionex ICS 3000 coupled to a PAD detector (Dionex), as described by Müller et al (2015, supra).

Ascorbic acid was measured spectrophotometrically at 265 nm (Agilent Cary 60 spectrophotometer) using a standard curve for quantification that was prepared using ascorbic acid concentrations ranging from 5 to 150 μM. A buffer-enzyme mixture was used as a blank.

### Results and Discussion

The effect of the AscA concentration (0-10 mM) and the oxygen concentration in the headspace (0-100%) on LPMO activity and saccharification yield on Avicel was examined using bottles as reaction vessels. The enzyme preparation used in these experiments was Cellic® CTec2, which is known to contain LPMOs (Müller et al., 2015, Biotechnology for Biofuels, 8, 187). A clear correlation between LPMO activity and glucose yield and between LPMO activity and the ascorbic acid concentration was observed (data not shown). Increasing the oxygen concentration in the headspace resulted in an almost linear correlation between the initial LPMO rate and the O₂ concentration (data not shown). However, the production of Glc4gemGlc (i.e. LPMO activity) ended after some time and this was reflected in a slowdown in glucose release (data not shown) suggesting inactivation of both cellulases and LPMOs, and degradation of already generated oxidized products. The higher the O₂ concentration, the earlier these inactivation processes seemed to happen.

Next a range of experiments with different combinations of AscA and H₂O₂, all carried out under anaerobic conditions in bottles were carried out. H₂O₂ was added stepwise.

Addition of both H₂O₂ and AscA led to a strong increase in LPMO activity (data not shown). It was also observed that when using 0.1 mM AscA and 200 μM H₂O₂, both become depleted due to unproductive reactions, limiting LPMO activity. While confirming the role of H₂O₂, these results also show that depletion of the reductant, e.g. by a surplus of H₂O₂, needs to be avoided. Accordingly, repetitive addition of AscA (0.1 mM) and H₂O₂ (200 μM) to a halted reaction that was started with 0.1 mM AscA and 200 μM H₂O₂, led to full recovery of LPMO activity (data not shown).

To probe the impact of the type of reducing agent, a range of reducing agents were tested. These reactions were initiated with 1 mM reducing agent and 200 μM H₂O₂, and then H₂O₂ was added (200 μM) every hour. All reactions showed the stepwise increase in oxidized sugars observed in the reaction with AscA, notably with higher production of oxidized sugars. Based on the final production of oxidized sugars the order of the reducing agents was: AscA (378 μM), gallic acid (461 μM), DTT (509 μM) and catechin (527 μM) (data not shown). Since the total addition of H₂O₂ was 800 μM, this corresponds to 47%, 58%, 64% and 66% of H₂O₂ being used to produce C4-oxidized sugars. Generally, regardless of the nature of the reductant, the results showed that controlled addition of both H₂O₂ and AscA (or only H₂O₂ if initial reductant concentrations are high) is highly beneficial for LPMO activity, compared to e.g. a standard reaction under aerobic conditions.

To assess the effects of continuous H₂O₂ administration, reactor experiments were set up using anaerobic conditions to obtain the best possible control of reaction conditions, for example by avoiding reactions between the reductant and O₂. The bioreactors operated with a liquid working volume of 900 mL, 10% (w/w) cellulosic substrate, 4 mg Cellic® CTec2 protein per gram dry matter, and feeding with different solutions of H₂O₂ (45-4500 μM) that were pumped in at a fixed rate of 600 μL h⁻¹. This yielded a H₂O₂ feed rate ranging from 30 to 3000 μM h⁻¹ (see Table 3). A linear relationship between H₂O₂ feed rate and apparent LPMO activity (Table 3, FIGS. 19, 20) was observed showing that the supply of H₂O₂ clearly was the limiting factor. Furthermore, for all tested feeding rates, except the highest, the first 6 hours of the reaction showed constant production of oxidized sugars (FIG. 19B). Importantly, the levels of detected Glc4gemGlc products corresponded to between 78 and 90% of the added H₂O₂, which is compatible with an expected 1:1 stoichiometry between H₂O₂ consumption and oxidative cleavage of cellulose, also meaning that no accumulation of H₂O₂ took place in the bioreactors during this first reaction phase. The “lacking” oxidized products can be attributed to the fact that, although the main LPMO activity in Cellic® CTec2 is C4-oxidizing, this enzyme cocktail is also known to form minor amounts of C1-oxidized products (gluconic acid; Cannella et al., 2012, Biotechnology for Biofuels, 5, 26), which could not be quantified in the experimental set-ups used here. All in all, the utilization of H₂O₂ by LPMOs for oxidative cleavage of cellulose seems to be very efficient. For technical reasons, the steady-state levels of H₂O₂ during the reactions depicted in FIG. 19 could not be determined. The stoichiometry of the reaction and the progress curves suggest that administered H₂O₂ is more or less immediately incorporated into LPMO-generated oxidized products. Thus, the steady-state level of H₂O₂ must be very low, possibly below 1 μM.

TABLE 3
Reactor feed setup, LPMO activity and ratio between added H₂O₂ and
generated oxidized products. Data from the experiments to which this Table applies
are depicted in FIGS. 19-21. The correlation between H₂O₂ feed rate and LPMO
activity is shown in FIG. 20

| H₂O₂ | H₂O₂ | LPMO | Cumulative | [Glc4gemGlc] | [Product]/[H₂O₂] | LPMO |
|---|---|---|---|---|---|---|
| in | feed | activity^{a}, | [H₂O₂] after | after | ratio after | activity^{-a}, |
| feed | rate | 1 h | 6 h | 6 h | 6 h | 6 h |
| (mM) | (μM h⁻¹) | (min⁻¹) | (μM)^{b} | (μM) | (%)^{c} | (min⁻¹) |
| 45 | 30 | 0.29 | 180 | 161.5 | 89.7 | 0.22 |
| 135 | 90 | 0.63 | 540 | 444.4 | 82.3 | 0.62 |
| 270 | 180 | 1.25 | 1080 | 947.5 | 87.7 | 1.32 |
| 450 | 300 | 2.02 | 1800 | 1394.2 | 77.5 | 1.94 |
| 900 | 600 | 4.13 | 3600 | 3018.2 | 83.8 | 4.19 |
| 4500 | 3000 | 15.05 | 18000 | 2004.7 | 11.1 | 2.78 |

^{a}LPMOs turnover rates were calculated based on the assumption that 15% (w/w) of the proteins in Cellic ® CTec2 is composed of LPMOs (Muller et al., 2015, supra). Avicel (10% w/w DM) was hydrolyzed with Cellic ® CTec2 (4 mg protein/g DM), yielding a total protein concentration of 400 mg/L, whereof LPMOs constitute 60 mg/L, which equals 2 μM (using an estimated molecular weight of 30 000 g/mol). Turnover rates were estimated from the 1 h and the 6 h points shown in FIG. 19B. Comparison of the 1 h and 6 h rates shows that product formation was almost linear with time in these six hours, except for the highest feed rate; see also FIG. 19B.
^{b}H₂O₂ concentration that would be measured in the bioreactor if it would accumulate, assuming that nothing is consumed or produced by the LPMOs or by redox side reactions with AscA.
^{c}This column lists the Glc4gemGlc concentration as percentage of the cumulative hypothetical H₂O₂ concentration (see footnote b), after 6 h reaction. In the presence of cellulases, as in Cellic ® CTec2, all C4-oxidized products are converted to Glc4gemGlc and C1-oxidized to gluconic acid and cellobionic acid. The by far dominating oxidized product generated by Cellic ® CTec2 is Glc4gemGlc.

At the highest feeding rate of 3000 μM h⁻¹ the initial production of oxidized sugars was very fast (see Table 3), but stopped after 2 hours incubation (FIG. 19B). This is likely due to inactivation of the LPMOs and not to AscA depletion since addition of fresh AscA (FIG. 21) failed to recover LPMO activity (FIG. 19B). This situation is similar to what was observed for the bottle experiments with 50 and 100% O₂ in the headspace (data not shown), where also inactivation of LPMOs seemed to take place.

The LPMO activity in the aerated bioreactor, which could be considered a “standard reaction”, was similar to the (low) activity in the bioreactor with the lowest feeding rate of 30 μM h⁻¹. Thus, major improvements of LPMO activity may be achieved relative to “standard conditions”, by feeding H₂O₂ at appropriate rates, i.e. higher than 30 μM h⁻¹.

Importantly, the LPMO activity correlated well with glucose release (FIG. 19A) for feed rates of 30, 90, 180, 300 and 600 μM h⁻¹ which after six hours gave 8, 14, 26, 27 and 33% higher glucose yields compared to anaerobic, H₂O₂-free conditions, respectively. However, the highest feed rate (3000 μM h⁻¹), which likely caused enzyme inactivation (FIG. 19B & Discussion above), led to a 6% reduction in glucose yield compared to anaerobic conditions. All reactions showed a gradual decrease in the AscA concentration, the rate of which was correlated with the H₂O₂ feed rate (FIG. 21). After six hours, all reactions still contained >0.78 mM AscA, except for the reaction with the 3000 μM h⁻¹ feed rate, where all AscA was depleted after 2 hours (FIG. 21). Note that in the reactions with feed rates varying from 90 to 600 μM h−1 the concentration of oxidized products is much higher than the reduction in the ascorbic acid concentration, confirming that the reductant is only needed in sub-stoichiometric amounts. Notably, the reaction with aeration and no added H₂O₂ also consumed AscA, which is to be expected since the generation of H₂O₂ from O₂ requires stoichiometric amounts of redox equivalents delivered by AscA and since generation of H₂O₂ drives the reaction.

To investigate the effects of H₂O₂ addition over a longer time period, experiments, using the same conditions as above and with Avicel (100 g/1) as substrate, were run for 48 h (data not shown). In this case three reactions were run with constant H₂O₂ addition (90, 300 and 600 μM h⁻¹), while two reactions were run with a variable feed rate, one reaction where the feed was gradually lowered (“Decrease”) and another where H₂O₂ feeding (300 μM h⁻¹) was started after 24 h (“Addition”).

The reaction with constant addition of 90 μM h⁻¹ H₂O₂ showed constant production of oxidized sugars over the full 48 hours and achieved a final glucose concentration of 69.2 g/L, i.e. 32% higher than in the anaerobic control reaction without H₂O₂ addition. The reactions constantly fed at 300 and 600 μM h⁻¹ gave fast initial production of glucose and Glc4gemGlc but collapsed after 18 h and 8 h, respectively. This collapse was reflected in attenuation of glucose production and attenuation of the production of oxidized products (the latter appear to be unstable in the presence of high levels of H₂O₂). This attenuation was associated with exhaustion of AscA. Thus, in addition to being needed for the reduction of the copper center, it seems that AscA, a well-known “anti-oxidant”, protects the enzymes from the damaging effect of excessive supply of H₂O₂. Addition of fresh AscA to these reactions neither restored glucose production nor the production of oxidized products, indicating that both cellulases and LPMOs had been inactivated.

Seeking further improvements, a reaction where the feed rate of H₂O₂ was gradually reduced was run. This proved to be the most efficient in terms of final glucose concentration (71.1 g/L; 35% higher than the anaerobic control reaction), data not shown.

Degradation of Industrial Lignocellulosic Substrates

The conversion of two different industrially relevant lignocellulosic biomasses, sulfite-pulped Norway spruce and steam exploded birch (SEB), was investigated using three constant H₂O₂ feed rates (90, 300 and 600 μM h⁻¹), under anaerobic conditions. The substrate concentration was 100 g dry matter per liter, as in the experiments with Avicel described above. In the control reaction, water was fed instead of H₂O₂. Reactions with sulfite-pulped Norway spruce were conducted in the presence of 1 mM AscA, since it had been shown previously that this lignin-poor substrate does not contain sufficient reducing power to potentiate LPMO activity. No AscA was added to the reaction with SEB, based on earlier data showing that this substrate can activate LPMOs (Müller et al., 2015, supra). Completely in line with the results reported for Avicel, above, the initial LPMO activity and the rate of glucose release correlated with the H₂O₂ feed rate (data not shown). The higher feed rates (300 and 600 μM h⁻¹) led to eventual inactivation of LPMOs, accompanied by retardation or even termination of the saccharification process (data not shown), as was previously observed for Avicel. Notably, the progress curves for the three substrates did show minor differences, in terms of LPMO rate and the time point of the onset of noticeable LPMO inactivation.

For sulfite-pulped Norway spruce, the highest glucose release after 48 h was obtained at 300 μM h⁻¹, where the yield, corresponding to 81% saccharification, was 46% higher compared to the control reaction. At a feed rate of 90 μM h⁻¹, production of Glc4gemGlc was stable during the whole incubation period as was the release of glucose, which increased by 37% relative to the control reaction.

Whilst the results for the different substrates followed the same trends there were some differences. While in the initial phase of the Avicel reaction >80% of the H₂O₂ ended up as Glc4gemGlc (Table 3), this fraction was 45-50% for Norway spruce and only 24-31% for SEB (data not shown). Thus, the higher the lignin content of the cellulosic substrates (Avicel<Norway spruce<birch wood), the less efficient was the integration of H₂O₂, although, notably, the saccharification yields obtained for steam exploded birch with feeding at 90 μM h⁻¹ were nevertheless among the highest ever reported for steam exploded lignocellulosic biomass.

Roles of LPMOs in Cellulose Degradation

The results presented above show that LPMO activity can be controlled and boosted by regulating the supply of H₂O₂, but also show the complex interplay between many factors including undesirable side reactions involving H₂O₂. The following provides the present understanding of the mechanisms involved. The LPMOs require a priming reduction to become active (from Cu(II) to Cu(I)). This reduction is carried out by a reductant, which can be a low molecular weight compound such as ascorbic acid, a protein (e.g. CDH) or a biomass-derived compound e.g. aromatic compounds from lignin. Once reduced, the enzyme can catalyze several catalytic cycles provided that H₂O₂, the co-substrate of the reaction, is supplied. It is important to note that the LPMOs will not carry out oxidation of the polysaccharide indefinitely, since they can desorb from the substrate and then may enter non-productive pathways leading to their oxidation back to the Cu(II) form. Known non-productive pathways are the reaction with O₂ in aerobic conditions, notably leading to the formation of H₂O₂, as well as enzyme self-destruction by reaction with H₂O₂ in the absence of substrate. Another side reaction concerns oxidation of the reductant, either by reaction with O₂ under aerobic conditions or by reaction with added H₂O₂ that is not consumed by the LPMO.

It is worth noting that the concentration of LPMOs in solution, and thus the potential for undesirable side reactions likely increases as the reaction proceeds and the substrate is degraded. The canonical glycoside hydrolases, i.e. the cellobiohydrolases, or CBHs, and the endoglucanases, or EGs, may play a role in maximizing binding of LPMO to the substrate by “peeling off” cellulose chains in regions where the crystalline structure has been disrupted by the LPMOs (Villares et al., 2017, Scientific Reports, 7:40262) and made susceptible for hydrolysis. The action of cellulases in these regions obviously results in substrate conversion towards glucose but also in re-generation of fresh crystalline surface to which the LPMOs can bind and carry out further oxidative chain cleavage (Eibinger et al., 2014, J. Biol. Chem., 289, 35929-35938). This interplay between the enzymes is of major importance when optimizing enzyme cocktails and processes.

### DISCUSSION

In recent years, several authors have reported that the simultaneous saccharification and fermentation (SFF) approach in biorefining may be less competitive than previously thought, because of competition for O₂ between LPMOs and microorganisms (Cannella and Jorgensen, 2014, Biotechnology and Bioengineering, 111, 59-68; Müller et al., 2017, Biotechnology and Bioengineering, 114, 552-559). In light of the above findings, the combination of O₂-dependent or anaerobic microorganisms with H₂O₂-dependent LPMO-containing cellulolytic cocktails can now be envisioned.

It is worth noting that the data presented above imply that, in the presence of substrate, the affinity of LPMOs for H₂O₂ must be very high. Even at pump rates as low as 30 μM h⁻¹, H₂O₂ is stoichiometrically and immediately incorporated into oxidized sugars. It is clear that the steady state concentration of H₂O₂ must be in the low- or sub-μM range.

This data allows for the adjustment of saccharificaton methods, e.g. methods in which enzymes and/or H₂O₂are added sequentially. Running bioreactors with feedback loops to continuously adjust the H₂O₂ feed and to minimize deleterious H₂O₂ accumulation is appropriate.

1. A method of enzymatically degrading a polysaccharide comprising contacting said polysaccharide with one or more lytic polysaccharide monooxygenase (LPMO), wherein said enzymatic degradation is carried out in a reaction in the presence of:
a) at least one reducing agent; and
b) hydrogen peroxide or a means which generates hydrogen peroxide, wherein the amount of hydrogen peroxide present during the degradation reaction is maintained in a concentration range at which the hydrogen peroxide acts as a co-substrate for said LPMO and said LPMO is inactivated by
(i) no more than 20% during a) the reaction time required to achieve 40% conversion of the polysaccharide or b) 4 hours of reaction time,
(ii) no more than 50% during a) the reaction time required to achieve 70% conversion of the polysaccharide or b) 12 hours of reaction time, or
(iii) no more than 20% when said LPMO is contacted with said concentration of hydrogen peroxide in the presence of said polysaccharide and reducing agent for 20 minutes.

2. The method as claimed in claim 1 wherein said method is conducted for at least 2 hours and/or achieves at least 40% conversion of the polysaccharide.

3. The method as claimed in claim 1 or 2 wherein said degradation results in the release of oxidized products, and the concentration of the reducing agent is at least ten fold lower than the concentration that would be necessary to achieve equivalent yields of oxidized products in reactions run under identical conditions but without hydrogen peroxide, wherein preferably said reducing agent is at a concentration of less than 200 μM, preferably less than 100 μM, especially preferably between 10 and 100 μM.

4. The method as claimed in any one of claims 1 to 3 wherein the hydrogen peroxide is maintained in said concentration range by changing the concentration of one or more of said (i) polysaccharide, (ii) one or more LPMO, (iii) at least one reducing agent, and (iv) hydrogen peroxide or means which generates hydrogen peroxide, during said reaction.

5. The method of any one of claims 1 to 4, wherein the concentration of hydrogen peroxide during the reaction is in the range of 0.01 to 200 μM, preferably 1 to 100 μM.

6. The method of any of claims 1 to 5 wherein hydrogen peroxide is supplied to the reaction at an average rate of 0.2 to 500 μM hydrogen peroxide per minute, preferably 0.5 to 20 μM hydrogen peroxide per minute.

7. The method as claimed in any one of claims 1 to 6 wherein the level of hydrogen peroxide is monitored one or more times during said degradation reaction.

8. The method as claimed in any one of claims 1 to 7 wherein said means which generates hydrogen peroxide is superoxide or a means which generates superoxide, wherein preferably said means which generates superoxide is a photochemical, chemical or enzymatic reaction.

9. The method as claimed in claim 8 wherein superoxide dismutase is added to the reaction to accelerate conversion of superoxide to hydrogen peroxide.

10. The method as claimed in any one of claims 1 to 9 wherein said means which generates hydrogen peroxide, which may comprise more than one part, is selected from:
(i) an enzyme and one or more components required for the activity of said enzyme;
(ii) a photoreactive compound and light; and
(iii) chemical means for generating hydrogen peroxide, preferably comprising more than one component which allows a chemical reaction that produces hydrogen peroxide to be conducted.

11. The method as claimed in claim 10 wherein said enzyme is an enzyme which generates hydrogen peroxide, preferably cellobiose dehydrogenase, a single domain flavoenzyme or superoxide dismutase.

12. The method as claimed in claim 10 wherein said photoreactive compound is chlorophyllin.

13. The method as claimed in claim 12 wherein said reducing agent is ascorbic acid, wherein preferably said ascorbic acid is used at a concentration of less than 2 mM, preferably less than 1 mM, e.g. from 0.01 to 0.2 mM.

14. The method as claimed in any one of claims 1 to 13 wherein said concentration range is maintained by
(i) addition and/or removal of said hydrogen peroxide or said means which generates hydrogen peroxide, or a part thereof;
(ii) addition and/or removal of a means to remove hydrogen peroxide;
(iii) addition or removal of said one or more LPMO;
(iv) addition and/or removal of said at least one reducing agent; and/or
(v) addition and/or removal of said polysaccharide,
during the degradation reaction.

15. The method as claimed in claim 14 wherein said means which generates hydrogen peroxide is an enzyme and one or more components required for the activity of said enzyme and said concentration range is maintained by addition and/or removal of said enzyme or one or more components required for its activity, wherein preferably said one or more components is selected from a co-factor or substrate for said enzyme.

16. The method as claimed in claim 14 wherein said means which generates hydrogen peroxide is a photoreactive compound and light and said concentration range is maintained by
(i) addition and/or removal of said photoreactive compound;
(ii) altering the duration and/or intensity and/or wavelength of light which irradiates the photoreactive compound;
and/or
(iii) addition and/or removal of said at least one reducing agent.

17. The method as claimed in any one of claims 1 to 16 wherein
(i) hydrogen peroxide or said means which generates hydrogen peroxide, or a part thereof;
(ii) a means to remove hydrogen peroxide;
(iii) said one or more LPMO;
(iv) said at least one reducing agent; and/or
(v) said polysaccharide,
is added to and/or removed from the reaction one or more times during the reaction, preferably two or more times during the reaction, preferably continuously.

18. The method as claimed in any one of claims 1 to 17 wherein hydrogen peroxide or said means which generates hydrogen peroxide, or a part thereof, or said means to remove hydrogen peroxide, is added to or removed from the reaction one or more, preferably two or more times during the reaction, preferably continuously.

19. The method as claimed in any one of claims 14 to 18 wherein said means to remove hydrogen peroxide is an enzyme, preferably a peroxidase, peroxyredoxin, peroxygenase or catalase.

20. The method as claimed in any one of claims 1 to 19 wherein the concentration of LPMO and/or reducing agent is changed during the degradation reaction by the addition or removal of said LPMO and/or reducing agent and/or a component which affects the concentration of said LPMO and/or reducing agent.

21. The method as claimed in any one of claims 1 to 20 wherein the concentration of dissolved molecular oxygen is reduced relative to the concentration of dissolved molecular oxygen present under aerobic conditions.

22. The method as claimed in any one of claims 1 to 21 wherein the method is conducted under anaerobic conditions.

23. The method as claimed in any one of claims 1 to 22 wherein said polysaccharide is cellulose or chitin.

24. The method as claimed in any one of claims 1 to 23 wherein said LPMO is an Auxiliary Activity family 9, 10, 11 or 13 protein, wherein preferably the LPMO is selected from ScLPMO10C, ScLPMO10B, SmLPMO10A, PcLPMO9D or TaGH61A.

25. The method as claimed in any one of claims 1 to 24 wherein two or more LPMOs are used in said method.

26. The method as claimed in any one of claims 1 to 25 wherein said reducing agent is selected from ascorbic acid, reduced glutathione, Fe(II)SO₄, LiAlH₄, NaBH₄, lignin or a fragment thereof, a cellobiose dehydrogenase, a phenol, a glucose-methanol-choline oxidoreductase, gallic acid or superoxide.

27. The method as claimed in any one of claims 1 to 26 wherein said polysaccharide is provided in a biomass.

28. The method as claimed in claim 27 wherein said biomass contains the reducing agent.

29. The method as claimed in any one of claims 1 to 28 wherein said one or more LPMO is present in the reaction in the amount of 0.005 to 2 g per kg of polysaccharide, preferably from 0.01 to 1 g per kg of polysaccharide.

30. The method as claimed in any one of claims 1 to 29 additionally comprising: contacting said polysaccharide (or the degradation product thereof) with one or more hydrolytic enzymes, preferably a cellulose hydrolase or chitin hydrolase, and optionally contacting said polysaccharide (or the degradation product thereof) with one or more enzymes selected from ß-glucosidases, hemicellulases, amylases, peroxidases, laccases or esterases, wherein preferably said enzymes are contacted with said polysaccharide simultaneously with said LPMO.

31. The method as claimed in claim 30 wherein said hydrolytic enzyme is chitinase or cellulase.

32. A method of producing soluble saccharides, wherein said method comprises degrading a polysaccharide by a method as defined in any one of claims 1 to 31 wherein said degradation releases said soluble saccharides and optionally isolating said soluble saccharides.

33. A method as claimed in claim 32 wherein said soluble saccharides are cellobiose and/or glucose and/or oligosaccharides thereof.

34. A method as claimed in claim 32 wherein said soluble saccharides are chitobiose and/or N-acetyl glucosamine and/or oligosaccharides thereof.

35. A method of producing an organic substance, comprising the steps of:
i) degrading a polysaccharide by a method as claimed in any one of claims 1 to 34 to produce a solution comprising soluble saccharides;
ii) fermenting said soluble saccharides, to produce said organic substance as the fermentation product; and optionally
iii) recovering said organic substance.

36. A method as claimed in claim 35 wherein said organic substance is an alcohol.

37. A method as claimed in claim 36 wherein said alcohol is ethanol.

38. Use of hydrogen peroxide as a co-substrate for a lytic polysaccharide monooxygenase to enzymatically degrade a polysaccharide.


SEQUENCE LISTING (8 sequences)

SEQ ID NO: 1 - DNA, 594 bp, Serratia marcescens
atgaacaaaacttcccgtaccctgctctctctgggcctgctgagcgcggccatgttcggcgtttcgcaacaggcgaatgcccacggttatgtcgaatcgccggccagccgcgcctatcagtgcaaactgcagctcaacacgcagtgcggcagcgtgcagtacgaaccgcagagcgtcgagggcctgaaaggcttcccgcaggccggcccggctgacggccatatcgccagcgccgacaagtccaccttcttcgaactggatcagcaaacgccgacgcgctggaacaagctcaacctgaaaaccggtccgaactcctttacctggaagctgaccgcgcgtcacagcaccaccagctggcgctatttcatcaccaagccgaactgggacgcttcgcagccgctgacccgcgcttcctttgacctgacgccgttctgccagttcaacgacggcggcgccatccctgccgcacaggtcacccaccagtgcaacataccggcagatcgcagcggttcgcacgtgatccttgccgtgtgggacatagccgacaccgctaacgccttctatcaggcgatcgacgtcaacctgagcaaataa

SEQ ID NO: 2 - protein, 197 aa, Serratia marcescens
MNKTSRTLLSLGLLSAAMFGVSQQANAHGYVESPASRAYQCKLQLNTQCGSVQYEPQSVEGLKGFPQAGPADGHIASADKSTFFELDQQTPTRWNKLNLKTGPNSFTWKLTARHSTTSWRYFITKPNWDASQPLTRASFDLTPFCQFNDGGAIPAAQVTHQCNIPADRSGSHVILAVWDIADTANAFYQAIDVNLSK

SEQ ID NO: 3 - DNA, 1095 bp, Streptomyces coelicolor
atggttcgacgcaccagactcctcaccctcgcggcggtactggccaccctgctcggctcgctcggcgtgacccttctgctcgggcaggggcgggccgaggcgcacggcgtggcgatgatgcccggctcccgcacctacctgtgccagctggacgccaagaccggcaccggcgccctcgacccgacgaaccccgcctgccaggccgccctcgaccagagcggggcgacggccctgtacaactggttcgccgtgctcgactccaacgcggggggccgcggcgccggttacgtgccggacggcaccctgtgcagcgccggcgaccgttccccgtacgacttctccgcctacaacgccgcccgctcggactggccccgcacgcacctgacgtcgggtgcgacgatcccggtggaatacagcaactgggcggcccaccccggggacttccgggtgtacctgaccaagccgggctggtcgcccacgtccgagctgggctgggacgacctggagctgatccagacggtgaccaacccgccccagcagggctcgccgggcaccgacgggggccactactactgggacctcgcgctgccctcgggccgctcgggcgacgcgttgatcttcatgcagtgggtgcgttcggacagccaggagaacttcttctcctgctcggacgtcgtcttcgacggcggcaacggagaggtcaccggcatccgcggttccgggagcaccccggacccggacccgacaccgaccccgacggacccgaccaccccgcccacgcacaccggctcctgcatggccgtgtactcggtggagaactcctggagcggcggcttccaggggtcggtcgaggtgatgaaccacggcaccgagccgctgaacggctgggccgtgcagtggcagccgggcggcgggaccacgctcggcggggtgtggaacggttcgctgaccagcggctccgacggtacggtcacggtccgcaacgtggaccacaaccgcgtcgtaccaccggacgggagcgtgaccttcggcttcaccgccacttcgacgggcaatgacttcccggtcgactcgatcggctgcgtggcaccctga

SEQ ID NO: 4 - protein, 364 aa, Streptomyces coelicolor
MVRRTRLLTLAAVLATLLGSLGVTLLLGQGRAEAHGVAMMPGSRTYLCQLDAKTGTGALDPTNPACQAALDQSGATALYNWFAVLDSNAGGRGAGYVPDGTLCSAGDRSPYDFSAYNAARSDWPRTHLTSGATIPVEYSNWAAHPGDFRVYLTKPGWSPTSELGWDDLELIQTVTNPPQQGSPGTDGGHYYWDLALPSGRSGDALIFMQWVRSDSQENFFSCSDVVFDGGNGEVTGIRGSGSTPDPDPTPTPTDPTTPPTHTGSCMAVYSVENSWSGGFQGSVEVMNHGTEPLNGWAVQWQPGGGTTLGGVWNGSLTSGSDGTVTVRNVDHNRVVPPDGSVTFGFTATSTGNDFPVDSIGCVAP

SEQ ID NO: 5 - DNA, 687 bp, Streptomyces coelicolor
tcagccgaagtcgacgtcgctgcacaggaagtaggtctggtccatgtgcgaggcctgccagatcgtgtagacgacgtgccggccggtcagtcccgaggtactcaccgggatctcgtagttctggctgggcccgtagctgcccgtgcgcgcgacctgctggagttctccccaggtcagggcctgggtggcggggtcgaagccctgcttggtgacatagaccaggaagtagtcggcgccgtggctggcctggtcgtggagcttgacggtgaagtcgtcggtgacgtccgtggtctgccacgggcccacggcgtcgagggagttgtagcgtccgctctcggtacggccgccgctgcacagctggccgtcggggacgaccgcctcgaagtcgccggccgagccgttgcggtagaggccgttccagttccacatggcgttggggtcgtcctgccacgcctgccagcacatggggtcctcgtcggccatggccgggttctggaagtcgtcgccccagcgttcccagcagccgtagttgcgggaggcggggtcgacgaccgagccgtgggccacggcggtgccgctccaggggatcagggtgagcaggaccgcggccagagtgctgaggacgagggtcgtcgcccgtcgggcccttccggcgagttgaattttggcgcgatcatgacaagtcat

SEQ ID NO: 6 - protein, 228 aa, Streptomyces coelicolor
MTCHDRAKIQLAGRARRATTLVLSTLAAVLLTLIPWSGTAVAHGSVVDPASRNYGCWERWGDDFQNPAMADEDPMCWQAWQDDPNAMWNWNGLYRNGSAGDFEAVVPDGQLCSGGRTESGRYNSLDAVGPWQTTDVTDDFTVKLHDQASHGADYFLVYVTKQGFDPATQALTWGELQQVARTGSYGPSQNYEIPVSTSGLTGRHVVYTIWQASHMDQTYFLCSDVDFG

SEQ ID NO: 7 - DNA, 708 bp, Phanerochaete chrysosporium
atgaaggccttcttcgccgttctcgctgtcgtctctgcgccattcgtcctgggacactacaccttccccgacttcattgagcctagcgggaccgtcaccggggattgggtctatgtccgcgagacccagaaccactacagtaacggaccggtcaccgatgtcacaagccccgagttccgctgctacgagctggacctgcagaacactgcaggacagacgcagaccgctacggtttccgccggtgacactgttggattcaaggcgaacagcgcaatttaccacccaggatacctcgacgtcatgatgtccccggcctccccagccgccaactcgcccgaggccggcactggccagacctggttcaagatctacgaggagaagccacagttcgagaatggccagctcgtttttgataccacgcagcaagaggtcacgttcacgatcccgaagagccttcccagtggccagtacctcctccgtatcgagcagatcgccctccacgtcgccagctcctatggcggtgcacagttctacatcgggtgcgctcaactcaacgtcgagaacggtggcaacggcaccccgggcccgctcgtgtcgatcccaggtgtctacaccggctacgagcccggtatcctcatcaacatctacaacctgccgaagaacttcaccggctacccggcgcctggccccgctgtctggcaaggatga

SEQ ID NO: 8 - protein, 235 aa, Phanerochaete chrysosporium
MKAFFAVLAVVSAPFVLGHYTFPDFIEPSGTVTGDWVYVRETQNHYSNGPVTDVTSPEFRCYELDLQNTAGQTQTATVSAGDTVGFKANSAIYHPGYLDVMMSPASPAANSPEAGTGQTWFKIYEEKPQFENGQLVFDTTQQEVTFTIPKSLPSGQYLLRIEQIALHVASSYGGAQFYIGCAQLNVENGGNGTPGPLVSIPGVYTGYEPGILINIYNLPKNFTGYPAPGPAVWQG